(19) United States
(12) Patent Application Publication
(10) Pub. No.: US 2012/0115903 A1
(43) Pub. Date: May 10, 2012

(54) Substituted Heteroaromatic Carboxamide and Urea Compounds as Vanilloid Receptor Ligands

(75) Inventors: Robert Frank, Aachen (DE); Thomas Christoph, Aachen (DE); Bernhard Lesch, Aachen (DE); Jeewoo Lee, Seoul (KR)

(73) Assignee: Gruenenthal GmbH, Aachen (DE)

(21) Appl. No.: 13/292,785

(22) Filed: Nov. 9, 2011

Related U.S. Application Data
(60) Provisional application No. 61/412,198, filed on Nov. 10, 2010.

(30) Foreign Application Priority Data
Nov. 10, 2010 (EP) 10 014 449.2

(51) Int. Cl. A61K 31/4155; C07D 401/14; A61K 31/4439; A61K 31/4196; C07D 471/04; A61K 31/437; C07D 413/12; A61K 31/423; A61K 31/4184; C07D 417/12; A61K 31/428; A61P 25/00; A61P 25/06; A61P 25/24; A61P 25/28; A61P 25/08; A61P 11/00; A61P 13/00; A61P 13/10; A61P 1/00; A61P 1/04; A61P 27/02; A61P 17/00; A61P 37/08; A61P 17/06; A61P 31/22; A61P 29/00; A61P 1/12; A61P 17/04; A61P 19/10; A61P 19/02; A61P 3/00; A61P 25/30; A61P 25/32; A61P 7/10; A61P 9/00; A61P 17/02; A61P 15/00; A61P 25/14; A61P 23/00; A61P 25/16; A61P 11/06; A61P 11/08; A61P 3/04; A61P 25/36; A61P 9/12; C07D 403/12

(52) U.S. Cl. 514/303; 548/364.7; 514/406; 546/275.4; 514/339; 548/266.4; 514/383; 546/119; 546/113; 514/300; 548/222; 514/375; 548/306.1; 514/395; 548/163; 514/367

(57) ABSTRACT

Substituted heteroaromatic carboxamide and urea compounds corresponding to formula (i)

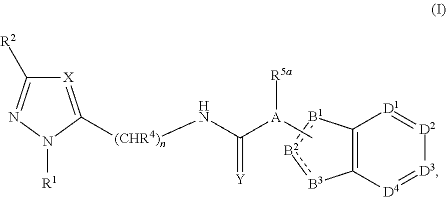

processes for the preparation thereof, pharmaceutical compositions containing these compounds and also a method of using these compounds in pharmaceutical compositions for treating or inhibiting pain and other conditions mediated at least in part via the vanilloid receptor 1.

### CROSS REFERENCE TO RELATED APPLICATIONS

This application claims priority from U.S. provisional patent application No. 61/412,198, filed Nov. 10, 2010. Priority is also claimed based on European patent application no. EP 10 014 449.2, filed Nov. 10, 2010. The disclosures of both priority applications are hereby incorporated by reference in their entirety.

### BACKGROUND OF THE INVENTION

The invention relates to substituted heteroaromatic carboxamide and urea derivatives, to processes for the preparation thereof, to pharmaceutical compositions containing these compounds and also to the use of these compounds for preparing pharmaceutical compositions.

The treatment of pain, in particular of neuropathic pain, is very important in medicine. There is a worldwide demand for effective pain therapies. The urgent need for action for a patient-focused and target-oriented treatment of chronic and non-chronic states of pain, this being understood to mean the successful and satisfactory treatment of pain for the patient, is also documented in the large number of scientific studies which have recently appeared in the field of applied analgesics or basic research on nociception.

The subtype 1 vanilloid receptor (VR1/TRPV1), which is often also referred to as the capsaicin receptor, is a suitable starting point for the treatment of pain, in particular of pain selected from the group consisting of acute pain, chronic pain, neuropathic pain and visceral pain, particularly preferably of neuropathic pain. This receptor is stimulated inter alia by vanilloids such as capsaicin, heat and protons and plays a central role in the formation of pain. In addition, it is important for a large number of further physiological and pathophysiological processes and is a suitable target for the therapy of a large number of further disorders such as, for example, migraine, depression, neurodegenerative diseases, cognitive disorders, states of anxiety, epilepsy, coughs, diarrhoea, pruritus, inflammations, disorders of the cardiovascular system, eating disorders, medication dependency, misuse of medication and in particular urinary incontinence.

There is a need for further compounds having comparable or better properties, not only with regard to affinity to vanilloid receptors 1 (VR1/TRPV1 receptors) per se (potency, efficacy).

Thus, it may be advantageous to improve the metabolic stability, the solubility in aqueous media or the permeability of the compounds. These factors can have a beneficial effect on oral bioavailability or can alter the PK/PD (pharmacokinetic/pharmacodynamic) profile; this can lead to a more beneficial period of effectiveness, for example.

A weak or non-existent interaction with transporter molecules, which are involved in the ingestion and the excretion of pharmaceutical compositions, is also to be regarded as an indication of improved bioavailability and at most low interactions of pharmaceutical compositions. Furthermore, the interactions with the enzymes involved in the decomposition and the excretion of pharmaceutical compositions should also be as low as possible, as such test results also suggest that at most low interactions, or no interactions at all, of pharmaceutical compositions are to be expected.

### SUMMARY OF THE INVENTION

It was therefore an object of the invention to provide novel compounds having advantages over the prior-art compounds. The compounds should be suitable in particular as pharmacological active ingredients in pharmaceutical compositions, preferably in pharmaceutical compositions for the treatment and/or inhibition of disorders or diseases which are mediated, at least in some cases, by vanilloid receptors 1 (VR1/TRPV1 receptors).

This object is achieved by the invention as described and claimed hereinafter.

It has surprisingly now been found that the substituted compounds of general formula (I), as indicated below, display outstanding affinity to the subtype 1 vanilloid receptor (VR1/TRPV1 receptor) and are therefore particularly suitable for the inhibition and/or treatment of disorders or diseases which are mediated, at least in some cases, by vanilloid receptors 1 (VR1/TRPV1

The present invention therefore relates to substituted compounds of general formula (I),

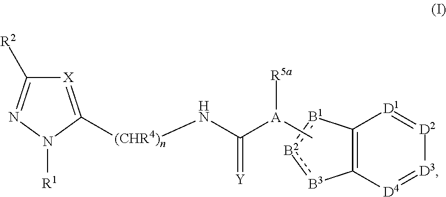

in which

- - - in each case represents the presence of precisely one double bond between B¹ and
B² or between B² and B³;
X represents CR³ or N,
   wherein R³ represents H; C₁₋₁₀ alkyl, saturated or unsaturated, branched or unbranched, unsubstituted or mono- or polysubstituted;
   A represents N or CR^{5b};
   n represents 1, 2, 3 or 4;
   Y represents O or S;
   R⁰ represents C₁₋₁₀ alkyl, saturated or unsaturated, branched or unbranched, unsubstituted or mono- or polysubstituted; C₃₋₁₀ cycloalkyl or heterocyclyl, respectively saturated or unsaturated, unsubstituted or mono- or polysubstituted; aryl or heteroaryl, respectively unsubstituted or mono- or polysubstituted; C₃₋₁₀ cycloalkyl or heterocyclyl bridged via C₁₋₈ alkyl, respectively saturated or unsaturated, unsubstituted or mono- or polysubstituted, wherein the alkyl chain can be respectively branched or unbranched, saturated or unsaturated, unsubstituted, mono- or polysubstituted; or aryl or heteroaryl bridged via C₁₋₈ alkyl, respectively unsubstituted or mono- or polysubstituted, wherein the alkyl chain can be respectively branched or unbranched, saturated or unsaturated, unsubstituted, mono- or polysubstituted;
   R¹ represents H; C₁₋₁₀ alkyl, saturated or unsaturated, branched or unbranched, unsubstituted or mono- or polysubstituted; C₃₋₁₀ cycloalkyl or heterocyclyl, respectively saturated or unsaturated, unsubstituted or mono- or polysubstituted; aryl or heteroaryl, respectively unsubstituted or mono- or polysubstituted; C₃₋₁₀ cycloalkyl or heterocyclyl bridged via C₁₋₈ alkyl, respectively saturated or unsaturated, unsubstituted or mono- or polysubstituted, wherein the alkyl chain can be respectively branched or unbranched, saturated or unsaturated, unsubstituted, mono- or polysubstituted; or aryl or heteroaryl bridged via C₁₋₈ alkyl, respectively unsubstituted or mono- or polysubstituted, wherein the alkyl chain can be respectively branched or unbranched, saturated or unsaturated, unsubstituted, mono- or polysubstituted; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰)(S(═O)₂—R⁰); or SCl₃;
   preferably represents C₁₋₁₀ alkyl, saturated or unsaturated, branched or unbranched, unsubstituted or mono- or polysubstituted; C₃₋₁₀ cycloalkyl or heterocyclyl, respectively saturated or unsaturated, unsubstituted or mono- or polysubstituted; aryl or heteroaryl, respectively unsubstituted or mono- or polysubstituted; C₃₋₁₀ cycloalkyl or heterocyclyl bridged via C₁₋₈ alkyl, respectively saturated or unsaturated, unsubstituted or mono- or polysubstituted, wherein the alkyl chain can be respectively branched or unbranched, saturated or unsaturated, unsubstituted, mono- or polysubstituted; or aryl or heteroaryl bridged via C₁₋₈ alkyl, respectively unsubstituted or mono- or polysubstituted, wherein the alkyl chain can be respectively branched or unbranched, saturated or unsaturated, unsubstituted, mono- or polysubstituted; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰)(S(═O)₂—R⁰; or SCl₃;
   R² represents H; R⁰; F; Cl; Br; I; CN; NO₂; OH; SH; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅;
   R⁴ represents H; F; Cl; Br; I; OH; C₁₋₁₀ alkyl, saturated or unsaturated, branched or unbranched, unsubstituted or mono- or polysubstituted;

R^{5a} represents H; OH; C₁₋₁₀ alkyl, saturated or unsaturated, branched or unbranched, unsubstituted or mono- or polysubstituted;

R^{5b} represents H or R⁰;

or R^{5a} and R^{5b} form together with the carbon atom connecting them a C₃₋₁₀ cycloalkyl or a heterocyclyl, respectively saturated or unsaturated, unsubstituted or mono- or polysubstituted;
B¹ represents C, CH, N, NR⁶, O or S;
B² represents C, CH, N, NR⁷, O or S;
B³ represents C, CH, N, NR⁸, O or S;
wherein 1 or 2 of the variables B¹, B² and B³ represent one of the afore mentioned heteroatoms or heteroatom groups;
D¹ represents N or CR⁹;
D² represents N or CR¹⁰;
D³ represents N or CR¹¹;
D⁴ represents N or CR¹²;
wherein 0, 1 or 2 of the variables D¹, D², D³ and D⁴ represent N;
R⁶, R⁷ and R⁸ each independently of one another represent H or C₁₋₄ alkyl, saturated, branched or unbranched, unsubstituted or mono- or polysubstituted;
R⁹, R¹⁰, R¹¹ and R¹² each independently of one another represent H; F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
in which “substituted alkyl”, “substituted heterocyclyl” and “substituted cycloalkyl” relate, with respect to the corresponding residues, to the substitution of one or more hydrogen atoms each independently of one another by F; Cl; Br; I; NO₂; CN; ═O; ═NH; ═N(OH); ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
in which “aryl substituted” and “heteroaryl substituted” relate, with respect to the corresponding residues, to the substitution of one or more hydrogen atoms each independently of one another by F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
in the form of the free compounds; the tautomers; the N-oxides; the racemate; the enantiomers, diastereomers, mixtures of the enantiomers or diastereomers or of an individual enantiomer or diastereomer; or in the form of the salts of physiologically compatible acids or bases.

The variables B¹, B², B³, D¹, D², D³ and D⁴ are selected in such a manner that the resulting partial structure

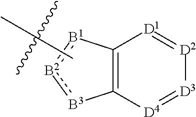

is aromatic.

Those skilled in the art understand that this aromatic partial structure may be attached via any of the positions corresponding to B¹, B² and B³.

The terms “alkyl” or “C₁₋₁₀ alkyl”, “C₁₋₈ alkyl”, “C₁₋₆ alkyl”, “C₁₋₄ alkyl” comprise in the sense of this invention acyclic saturated or unsaturated aliphatic hydrocarbon residues, i.e. C₁₋₁₀ aliphatic residues, C₁₀ aliphatic residues, C₁₋₆ aliphatic residues and C₁₋₄ aliphatic residues, which can be respectively branched or unbranched and also unsubstituted or mono- or polysubstituted, containing 1 to 10 or 1 to 8 or 1 to 6 or 1 to 4 carbon atoms, i.e. C₁₋₁₀ alkanyls, C₂₋₁₀ alkenyls and C₂₋₁₀ alkinyls or C₁₋₈ alkanyls, C₂₋₈ alkenyls and C₂₋₈ alkinyls or C₁₋₆ alkanyls, C₂₋₆ alkenyls and C₂₋₆ alkinyls or C₁₋₄ alkanyls, C₂₋₄ alkenyls and C₂₋₄ alkinyls. In this case, alkenyls comprise at least one C—C double bond and alkinyls comprise at least one C—C triple bond. Preferably, alkyl is selected from the group comprising methyl, ethyl, n-propyl, 2-propyl, n-butyl, isobutyl, sec.-butyl, tert.-butyl, n-pentyl, isopentyl, neopentyl, n-hexyl, n-heptyl, n-octyl, n-nonyl, n-decyl, ethenyl (vinyl), ethinyl, propenyl (—CH₂CH═CH₂, —CH═CH—CH₃, —C(═CH₂)—CH₃), propinyl (—CH—C≡CH, —C≡C—CH₃), butenyl, butinyl, pentenyl, pentinyl, hexenyl and hexinyl, heptenyl, heptinyl, octenyl, octinyl, nonenyl, noninyl, decenyl and decinyl.

The terms “cycloalkyl” or “C₃₋₁₀ cycloalkyl” mean for the purposes of this invention cyclic aliphatic (cycloaliphatic) hydrocarbons containing 3, 4, 5, 6, 7, 8, 9 or 10 carbon atoms, i.e. C₃₋₁₀-cycloaliphatic residues, wherein the hydrocarbons can be saturated or unsaturated (but not aromatic), unsubstituted or mono- or polysubstituted. The cycloalkyl can be bound to the respective superordinate general structure via any desired and possible ring member of the cycloalkyl residue. The cycloalkyl residues can also be condensed with further saturated, (partially) unsaturated, (hetero)cyclic, aromatic or heteroaromatic ring systems, i.e. with cycloalkyl, heterocyclyl, aryl or heteroaryl which can in turn be unsubstituted or mono- or polysubstituted. The cycloalkyl residues can furthermore be singly or multiply bridged such as, for example, in the case of adamantyl, bicyclo[2.2.1]heptyl or bicyclo[2.2.2]octyl. Preferably, cycloalkyl is selected from the group comprising cyclopropyl, cyclobutyl, cyclopentyl, cyclohexyl, cycloheptyl, cyclooctyl, cyclononyl, cyclodecyl, adamantyl,

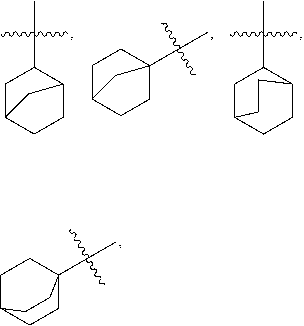

cyclopentenyl, cyclohexenyl, cycloheptenyl and cyclooctenyl.

The terms “heterocyclyl” or “heterocycloalkyl” comprise aliphatic saturated or unsaturated (but not aromatic) cycloalkyls having three to ten, i.e. 3, 4, 5, 6, 7, 8, 9 or 10, ring members, in which at least one, if appropriate also two or three carbon atoms are replaced by a heteroatom or a heteroatom group each selected independently of one another from the group consisting of O, S, N, NH and N(C₁₋₈ alkyl), preferably N(CH₃), wherein the ring members can be unsubstituted or mono- or polysubstituted. Heterocyclyls are thus heterocycloaliphatic residues. The heterocyclyl can be bound to the superordinate general structure via any desired and possible ring member of the heterocyclyl residue. The heterocyclyl residues can therefore be condensed with further saturated, (partially) unsaturated (hetero)cyclic or aromatic or heteroaromatic ring systems, i.e. with cycloalkyl, heterocyclyl, aryl or heteroaryl which can in turn be unsubstituted or mono- or polysubstituted. Heterocyclyl residues selected from the group comprising azetidinyl, aziridinyl, azepanyl, azocanyl, diazepanyl, dithiolanyl, dihydroquinolinyl, dihydropyrrolyl, dioxanyl, dioxolanyl, dioxepanyl, dihydroindenyl, dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, pyrrolidinyl, piperazinyl, 4-methylpiperazinyl, piperidinyl, pyrazolidinyl, pyranyl, tetrahydropyrrolyl, tetrahydropyranyl, tetrahydroquinolinyl, tetrahydroisoquinolinyl, tetrahydroindolinyl, tetrahydrofuranyl, tetrahydropyridinyl, tetrahydrothiophenyl, tetrahydropyridoindolyl, tetrahydronaphthyl, tetrahydrocarbolinyl, tetrahydroisoxa-zolopyridinyl, thiazolidinyl and thiomorpholinyl are preferred.

The term “aryl” means in the sense of this invention aromatic hydrocarbons having up to 14 ring members, including phenyls and naphthyls. Each aryl residue can be unsubstituted or mono- or polysubstituted, wherein the aryl substituents can be the same or different and in any desired and possible position of the aryl. The aryl can be bound to the superordinate general structure via any desired and possible ring member of the aryl residue. The aryl residues can also be condensed with further saturated, (partially) unsaturated, (hetero)cyclic, aromatic or heteroaromatic ring systems, i.e. with cycloalkyl, heterocyclyl, aryl or heteroaryl which can in turn be unsubstituted or mono- or polysubstituted. Examples of condensed aryl residues are benzodioxolanyl and benzodioxanyl. Preferably, aryl is selected from the group containing phenyl, 1-naphthyl and 2-naphthyl which can be respectively unsubstituted or mono- or polysubstituted. A particularly preferred aryl is phenyl, unsubstituted or mono- or polysubstituted.

The term “heteroaryl” represents a 5 or 6-membered cyclic aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted; in the case of substitution on the heteroaryl, the substituents can be the same or different and be in any desired and possible position of the heteroaryl. The binding to the superordinate general structure can be carried out via any desired and possible ring member of the heteroaryl residue. The heteroaryl can also be part of a bi- or polycyclic system having up to 14 ring members, wherein the ring system can be formed with further saturated, (partially) unsaturated, (hetero)cyclic or aromatic or heteroaromatic rings, i.e. with cycloalkyl, heterocyclyl, aryl or heteroaryl which can in turn be unsubstituted or mono- or polysubstituted. It is preferable for the heteroaryl residue to be selected from the group comprising benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl or triazinyl. Pyridyl may be particularly preferred.

The terms “aryl, heteroaryl, heterocyclyl or cycloalkyl bridged via C₁₋₄ alkyl or C₁₋₈ alkyl” mean in the sense of the invention that C₁₋₄ alkyl or C₁₋₈ alkyl and aryl or heteroaryl or heterocyclyl or cycloalkyl have the above-defined meanings and the aryl or heteroaryl or heterocyclyl or cycloalkyl residue is bound to the respective superordinate general structure via a C₁₋₄ alkyl or a C₁₋₈ alkyl group. The alkyl chain of the alkyl group can in all cases be branched or unbranched, unsubstituted or mono- or polysubstituted. The alkyl chain of the alkyl group can furthermore be in all cases saturated or unsaturated, i.e. can be an alkylene group, i.e. a C₁₋₄ alkylene group or a C₁₋₈ alkylene group, an alkenylene group, i.e. a C₂₋₄ alkenylene group or a C₂₋₈ alkenylene group, or an alkinylene group, i.e. a C₂₋₄ alkinylene group or a C₂₋₈ alkinylene group. Preferably, C₁₋₄ alkyl is selected from the group comprising —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —C≡C—, —C≡C—CH₂—, —C≡C—CH₂—CH₂—, —C≡C—CH(CH₃)—, —CH₂—C≡C—CH₂— and —C≡C—C≡C— and C₁₋₈ alkyl is selected from the group comprising —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂—, —CH₂—(CH₂)₄—CH₂—, —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂—, —CH═CH₂—CH—CH═CH₂—, —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C≡C—CH₂— and —C≡C—CH₂—C≡C—.

In relation to “alkyl”, “heterocyclyl” and “cycloalkyl”, the term “mono- or polysubstituted” refers in the sense of this invention to the single or multiple, for example double, triple or quadruple, substitution of one or more hydrogen atoms each independently of one another by substituents selected from the group of F; Cl; Br; I; NO₂; CN; ═O; ═NH; ═N(OH); ═C(NH₂)₂; CF₃; CF₂H; CFH₂; CF₂Cl; CFCl₂; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—W; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂; wherein the term “polysubstituted residues” refers to residues of the type that are polysubstituted, for example di-, tri- or tetrasubstituted, either on different or on the same atoms, for example trisubstituted on the same C atom, as in the case of CF₃ or CH₂CF₃, or at various points, as in the case of CH(OH)—CH═CH—CHCl₂. A substituent can if appropriate for its part in turn be mono- or polysubstituted. The multiple substitution can be carried out using the same or using different substituents.

Preferred “alkyl”, “heterocyclyl” and “cycloalkyl” substituents are selected from the group of F; Cl; Br; I; NO₂; CF₃; CN; ═O; ═NH; R⁰; C(═O)(R⁰ or H); C(═O)O(R⁰ or H); C(═O)N(R⁰ or H)₂; OH; OR⁰; O—C(═O)—R⁰; O—(C₁₋₈ alkyl)-OH; O—(C₁₋₈ alkyl)-O—C₁₋₈ alkyl; OCF₃; N(R⁰ or H)₂; N(R⁰ or H)—C(═O)—R⁰; N(R⁰ or H)—C(═O)—N(R⁰ or H)₂; SH; SCF₃; SR⁰; S(═O)₂R⁰; S(═O)₂O(R⁰ or H) and S(═O)₂—N(R⁰ or H)₂.

Particularly preferred “alkyl”, “heterocyclyl” and “cycloalkyl” substituents are selected from the group consisting of F; Cl; Br; I; NO₂; CF₃; CN; ═O; C₁₋₈ alkyl; aryl; heteroaryl; C₃₋₁₀ cycloalkyl; heterocyclyl; aryl, heteroaryl, C₃₋₁₀ cycloalkyl or heterocyclyl bridged via C₁₋₈ alkyl; CHO; C(═O)C₁₋₈ alkyl; C(═O)aryl; C(═O)heteroaryl; CO₂H; C(═O)O—C₁₋₈ alkyl; C(═O)O-aryl; C(═O)O-heteroaryl; CONH₂; C(═O)NH—C₁₋₈ alkyl; C(═O)N(C₁₋₈ alkyl)₂; C(═O)NH-aryl; C(═O)N(aryl)₂; C(═O)NH-heteroaryl; C(═O)N(heteroaryl)₂; C(═O)N(C₁₋₈ alkyl)(aryl); C(═O)N(C₁₋₈ alkyl)(heteroaryl); C(═O)N(heteroaryl)(aryl); OH; O—C₁₋₈ alkyl; OCF₃; O—(C₁₋₈ alkyl)-OH; O—(C₁₋₈ alkyl)-O—C₁₋₈ alkyl; O-benzyl; O-aryl; O-heteroaryl; O—C(═O)C₁₋₈ alkyl; O—C(═O)aryl; O—C(═O)heteroaryl; NH₂, NH—C₁₋₈ alkyl; N(C₁₋₈ alkyl)₂; NH—C(═O)C₁₋₈ alkyl; NH—C(═O)-aryl; NH—C(═O)-heteroaryl; SH; S—C₁₋₈ alkyl; SCF₃; S-benzyl; S-aryl; S-heteroaryl; S(═O)₂C₁₋₈ alkyl; S(═O)₂ aryl; S(═O)₂ heteroaryl; S(═O)₂OH; S(═O)₂O—C₁₋₈ alkyl; S(═O)₂O-aryl; S(═O)₂O-heteroaryl; S(═O)₂—NH—C₁₋₈ alkyl; S(═O)₂—NH-aryl; and S(═O)₂—NH—C₁₋₈ heteroaryl.

In relation to “aryl” and “heteroaryl”, the term “mono- or polysubstituted” refers in the sense of this invention to the single or multiple, for example double, triple or quadruple, substitution of one or more hydrogen atoms of the ring system each independently of one another by substituents selected from the group of F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—OH; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂, on one or if appropriate different atoms, wherein a substituent can if appropriate for its part in turn be mono- or polysubstituted. The multiple substitution is carried out using the same or using different substituents.

Preferred “aryl” and “heteroaryl” substituents are F; Cl; Br; I; NO₂; CF₃; CN; R⁰; C(═O)(R⁰ or H); C(═O)O(R⁰ or H); C(═O)N(R⁰ or H)₂; OH; OR⁰; O—C(═O)—R⁰; O—(C₁₋₈ alkyl)-O—C₁₋₈ alkyl; OCF₃; N(R⁰ or H)₂; N(R⁰ or H)—C(═O)—R⁰; N(R⁰ or H)—C(═O)—N(R⁰ or H)₂; SH; SCF₃; SR⁰; S(═O)₂R⁰; S(═O)₂O(R⁰ or H); S(═O)₂—N(R⁰ or H)₂.

Particularly preferred “aryl” and “heteroaryl” substituents are selected from the group consisting of F; Cl; Br; I; NO₂; CF₃; CN; C₁₋₈ alkyl; aryl; heteroaryl; C₃₋₁₀ cycloalkyl; heterocyclyl; C₁₋₈ alkyl-bridged aryl, heteroaryl, C₃₋₁₀ cycloalkyl or heterocyclyl; CHO; C(═O)C₁₋₈ alkyl; C(═O)aryl; C(═O)heteroaryl; CO₂H; C(═O)O—C₁₋₈ alkyl; C(═O)O-aryl; C(═O)O-heteroaryl; CONH₂; C(═O)NH—C₁₋₈ alkyl; C(═O)N(C₁₋₈ alkyl)₂; C(═O)NH-aryl; C(═O)N(aryl)₂; C(═O)NH-heteroaryl; C(═O)N(heteroaryl)₂; C(═O)N(C₁₋₈ alkyl)(aryl); C(═O)N(C₁₋₈ alkyl)(heteroaryl); C(═O)N(heteroaryl)(aryl); OH; O—C₁₋₈ alkyl; OCF₃; O—(C₁₋₈ alkyl)-OH; O—(C₁₋₈ alkyl)-O—C₁₋₈ alkyl; O-benzyl; O-aryl; O-heteroaryl; O—C(═O)C₁₋₈ alkyl; O—C(═O)aryl; O—C(═O)heteroaryl; NH₂; NH—C₁₋₈ alkyl; N(C₁₋₈ alkyl)₂; NH—C(═O)C₁₋₈ alkyl; NH—C(═O)-aryl; NH—C(═O)-heteroaryl; SH; S—C₁₋₈ alkyl; SCF₃; S-benzyl; S-aryl; S-heteroaryl; S(═O)₂C₁₋₈ alkyl; S(═O)₂aryl; S(═O)₂ heteroaryl; S(═O)₂OH; S(═O)₂O—C₁₋₈ alkyl; S(═O)₂O-aryl; S(═O)₂O-heteroaryl; S(═O)₂—NH—C₁₋₈ alkyl; S(═O)₂—NH-aryl; S(═O)₂—NH—C₁₋₈ heteroaryl.

Even more particularly preferred substituents for “aryl” and “heteroaryl” are selected from the group consisting of F; Cl; Br; CF₃; OCF₃; CN; C₁₋₄ alkyl, O—C₁₋₄-alkyl and C₃₋₆ cycloalkyl.

The compounds according to the invention are defined by substituents, for example by R¹, R² and R³ (1^{st} generation substituents) which are for their part if appropriate substituted (2^{nd} generation substituents). Depending on the definition these substituents of the substituents can for their part be resubstituted (3^{rd} generation substituents). If, for example, R¹=aryl (1^{st} generation substituent), then aryl can for its part be substituted, for example with C₁₋₈ alkyl (2^{nd} generation substituent). This produces the functional group aryl-C₁₋₈ alkyl. C₁₋₈ alkyl can then for its part be resubstituted, for example with Cl (3^{rd} generation substituent). Overall, this then produces the functional group aryl-C₁₋₈ alkyl-Cl.

However, in a preferred embodiment, the 3^{rd} generation substituents may not be resubstituted, i.e. there are then no 4^{th} generation substituents.

In another preferred embodiment, the 2^{nd} generation substituents may not be resubstituted, 3^{rd} i.e. there are then not even any 0.5 generation substituents. In other words, in this embodiment, in the case of general formula (I), for example, the functional groups for R¹ to R¹² can each if appropriate be substituted; however, the respective substituents may then for their part not be resubstituted.

In some cases, the compounds according to the invention are defined by substituents which are or carry an aryl or heteroaryl residue, respectively unsubstituted or mono- or polysubstituted, or which form together with the carbon atom(s) or heteroatom(s) connecting them, as the ring member or as the ring members, a ring, for example an aryl or heteroaryl, respectively unsubstituted or mono- or polysubstituted. Both these aryl or heteroaryl residues and the aromatic ring systems formed in this way can if appropriate be condensed with C₃₋₁₀ cycloalkyl or heterocyclyl, respectively saturated or unsaturated, or with aryl or heteroaryl, i.e. with a C₃₋₁₀ cycloalkyl such as cyclopentyl or a heterocyclyl such as morpholinyl, or an aryl such as phenyl or a heteroaryl such as pyridyl, wherein the C₃₋₁₀ cycloalkyl or heterocyclyl residues, aryl or heteroaryl residues condensed in this way can for their part be respectively unsubstituted or mono- or polysubstituted.

In some cases, the compounds according to the invention are defined by substituents which are or carry a C₃₋₁₀ cycloalkyl or heterocyclyl residue, respectively unsubstituted or mono- or polysubstituted, or which form together with the carbon atom(s) or heteroatom(s) connecting them, as the ring member or as the ring members, a ring, for example a C₃₋₁₀ cycloalkyl or heterocyclyl, respectively unsubstituted or mono- or polysubstituted. Both these C₃₋₁₀ cycloalkyl or heterocyclyl residues and the aliphatic ring systems formed can if appropriate be condensed with aryl or heteroaryl or with C₃₋₁₀ cycloalkyl or heterocyclyl, i.e. with an aryl such as phenyl or a heteroaryl such as pyridyl or a C₃₋₁₀ cycloalkyl such as cyclohexyl or a heterocyclyl such as morpholinyl, wherein the aryl or heteroaryl residues or C₃₋₁₀ cycloalkyl or heterocyclyl residues condensed in this way can for their part be respectively unsubstituted or mono- or polysubstituted.

Within the scope of the present invention, the symbol

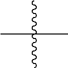

used in the formulae denotes a link of a corresponding residue to the respective superordinate general structure.

The term “(R⁰ or H)” within a residue means that R⁰ and H can occur within this residue in any possible combination. Thus, for example, the residue “N(R⁰ or H)₂” can represent “NH₂”, “NHR⁰” and)“N(R⁰)₂”. If, as in the case of)“N(R⁰)₂”, R⁰ occurs multiply within a residue, then R⁰ can respectively have the same or different meanings: in the present example of)“N(R⁰)₂”, R⁰ can for example represent aryl twice, thus producing the functional group “N(aryl)₂”, or R⁰ can represent once aryl and once C₁₋₁₀ alkyl, thus producing the functional group “N(aryl)(C₁₋₁₀ alkyl)”.

If a residue occurs multiply within a molecule, such as for example the residue R⁰, then this residue can have respectively different meanings for various substituents: if, for example, both R¹═R⁰ and R²═R⁰, then R⁰ can represent R¹=aryl and R⁰ can represent R²═C₁₋₁₀ alkyl.

The term “salt formed with a physiologically compatible acid” refers in the sense of this invention to salts of the respective active ingredient with inorganic or organic acids which are physiologically compatible—in particular when used in human beings and/or other mammals. Hydrochloride is particularly preferred. Examples of physiologically compatible acids are: hydrochloric acid, hydrobromic acid, sulphuric acid, methanesulphonic acid, p-toluenesulphonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulphonic acid, nicotinic acid, 2, 3 or 4-aminobenzoic acid, 2,4,6-trimethylbenzoic acid, α-lipoic acid, acetyl glycine, hippuric acid, phosphoric acid, aspartic acid. Citric acid and hydrochloric acid are particularly preferred.

Physiologically compatible salts with cations or bases are salts of the respective compound—as an anion with at least one, preferably inorganic, cation—which are physiologically compatible—in particular when used in human beings and/or other mammals. Particularly preferred are the salts of the alkali and alkaline earth metals but also ammonium salts [NHₓR₄₋ₓ]⁺, in which x=0, 1, 2, 3 or 4 and R represents a branched or unbranched C₁₋₄ alkyl residue, in particular (mono-) or (di)sodium, (mono-) or (di)potassium, magnesium or calcium salts.

In preferred embodiments of the compounds according to the invention of general formula (I), n represents 1, 2, 3 or 4, preferably 1, 2 or 3, particularly preferably 1 or 2, most particularly preferably 1.

In a further preferred embodiment of the compounds according to the invention of general formula (I), the residue

R¹ represents H; C₁₋₁₀ alkyl, C(═O)—C₁₋₁₀ alkyl, C(═O)—NH—C₁₋₁₀ alkyl, C(═O)—N(C₁₋₁₀ alkyl)₂, O—C₁₋₁₀ alkyl, S—C₁₋₁₀ alkyl, NH(C₁₋₁₀ alkyl), N(C₁₋₁₀ alkyl)₂, NH—S(═O)₂—C₁₋₁₀ alkyl, N(C₁₋₁₀ alkyl)-S(═O)₂—C₁₋₁₀ alkyl, S(═O)₂—C₁₋₁₀ alkyl, S(═O)₂—NH—C₁₋₁₀ alkyl, S(═O)₂—N(C₁₋₁₀ alkyl)₂, in which C₁₋₁₀ alkyl can be respectively saturated or unsaturated, branched or unbranched, unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
preferably represents C₁₋₁₀ alkyl, C(═O)—C₁₋₁₀ alkyl, C(═O)—NH—C₁₋₁₀ alkyl, C(═O)—N(C₁₋₁₀ alkyl)₂, O—C₁₋₁₀ alkyl, S—C₁₋₁₀ alkyl, NH(C₁₋₁₀ alkyl), N(C₁₋₁₀ alkyl)₂, NH—S(═O)₂—C₁₋₁₀ alkyl, N(C₁₋₁₀ alkyl)-S(═O)₂C₁₋₁₀ alkyl, S(═O)₂—C₁₋₁₀ alkyl, S(═O)₂—NH—C₁₋₁₀ alkyl, S(═O)₂—N(C₁₋₁₀ alkyl)₂, in which C₁₋₁₀ alkyl can be respectively saturated or unsaturated, branched or unbranched, unsubstituted or mono- or polysubstituted with one or more substituents each selected independently of one another from the group consisting of F, Cl, Br, I, NO₂, CN, OH, ═O, C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
or C₃₋₁₀ cycloalkyl or heterocyclyl, respectively saturated or unsaturated, unsubstituted or mono- or polysubstituted with one or more substituents each selected independently of one another from the group consisting of F, Cl, Br, I, NO₂, CN, OH, O, O—C₁₋₄ alkyl, OCF₃, CF₃, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
or C₃₋₁₀ cycloalkyl or heterocyclyl bridged via C₁₋₈ alkyl, respectively saturated or unsaturated, unsubstituted or mono- or polysubstituted with one or more substituents each selected independently of one another from the group consisting of F, Cl, Br, I, NO₂, CN, OH, ═O, C—C₁₋₄ alkyl, OCF₃, CF₃, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; wherein the alkyl chain can be respectively branched or unbranched, saturated or unsaturated, unsubstituted, mono- or polysubstituted with one or more substituents each selected independently of one another from the group consisting of F, Cl, Br; I, OH and O—C₁₋₄ alkyl;
or C(═O)—C₃₋₁₀ cycloalkyl, O—C₃₋₁₀ cycloalkyl, S—C₃₋₁₀ cycloalkyl, NH—C(═O)-cycloalkyl, NH—C(═O)-heterocyclyl, respectively saturated or unsaturated, unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
or aryl, heteroaryl, C(═O)-aryl, C(═O)-heteroaryl, O-aryl, O-heteroaryl, NH(aryl), N(aryl)₂, NH(heteroaryl), N(heteroaryl)₂, NH—C(═O)-aryl, NH—C(═O)-heteroaryl, NH—S(═O)₂-aryl, NH—S(═O)₂-heteroaryl, S(═O)₂-aryl, S(═O)₂-heteroaryl or aryl or heteroaryl bridged via C₁₋₈ alkyl, wherein aryl and heteroaryl can be respectively unsubstituted or mono- or polysubstituted with one or more substituents each selected independently of one another from the group consisting of F, Cl, Br, I, NO₂, CN, OH, C₁₋₄ alkyl, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl, and wherein if appropriate the alkyl chain can be respectively branched or unbranched, saturated or unsaturated, unsubstituted, mono- or polysubstituted with one or more substituents each selected independently of one another from the group consisting of F, Cl, Br, I, OH and O—C₁₋₄ alkyl.

In another preferred embodiment of the compounds according to the invention of general formula (I), the residue

R¹ represents substructure (T1)

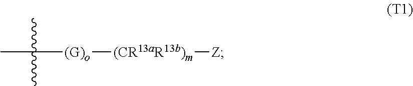

in which
G represents C(═O), O, S, S(═O)₂, NH—C(═O) or NR¹⁴;
wherein R¹⁴ represents H; C₁₋₈ alkyl or S(═O)₂—C₁₋₈ alkyl, in which C₁₋₈ alkyl can be respectively saturated or unsaturated, branched or unbranched, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
   o represents 0 or 1;
   R^{13a} and R^{13b} each independently of one another represent H; F; Cl; Br; I; NO₂; CF₃; CN; OH; OCF₃; NH₂; C₁₋₄ alkyl, O—C₁₋₄ alkyl, NH—C₁₋₄ alkyl, N(C₁₋₄ alkyl)₂, in which C₁₋₄ alkyl can be respectively saturated or unsaturated, branched or unbranched, unsubstituted or mono- or polysubstituted with one or more substituents each selected independently of one another from the group consisting of F, Cl, Br, I, O—C₁₋₄ alkyl, OH and OCF₃;
   on the condition that if R^{13a} and R^{13b} are bound to the same carbon atom, only one of the substituents R^{13a} and R^{13b} can represent OH; OCF₃; NH₂; O—C₁₋₄ alkyl, NH—C₁₋₄ alkyl or N(C₁₋₄ alkyl)₂;
   m represents 0, 1, 2, 3 or 4;
   Z represents C₁₋₄ alkyl, saturated or unsaturated, branched or unbranched, unsubstituted or mono- or polysubstituted with one or more substituents each selected independently of one another from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ and S(═O)₂OH; C₃₋₁₀ cycloalkyl or heterocyclyl, respectively saturated or unsaturated, unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, SH, S—C₁₋₄ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, O—C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; aryl or heteroaryl, respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

If m≠0, then the residues R^{13a} and R^{13b} can, taking account of the foregoing condition, both on the same carbon atom and on different carbon atoms, each independently of one another represent H; F; Cl; Br; I; NO₂; CF₃; CN; OH; OCF₃; NH₂; C₁₋₄ alkyl, O—C₁₋₄ alkyl, NH—C₁₋₄ alkyl, N(C₁₋₄ alkyl)₂, in which C₁₋₄ alkyl can be respectively saturated or unsaturated, branched or unbranched, unsubstituted or mono- or polysubstituted with one or more substituents each selected independently of one another from the group consisting of F, Cl, Br, I, O—C₁₋₄ alkyl, OH and OCF₃.

Preferably, the residue

G represents C(═O), O, S, S(═O)₂, NH—C(═O) or NR¹⁴,
wherein R¹⁴ represents H; methyl; ethyl; n-propyl; isopropyl; n-butyl; sec.-butyl; tert.-butyl; S(═O)₂-methyl; S(═O)₂-ethyl;
   o represents 0 or 1;
   R^{13a} and R^{13b} each independently of one another represent H; F; Cl; Br; I; NO₂; CF₃; CN; methyl; ethyl; n-propyl; isopropyl; n-butyl; sec.-butyl; tert.-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂; or N(methyl)(ethyl);
on the condition that if R^{13a} and R^{13b} are bound to the same carbon atom, only one of the substituents R^{13a} and R^{13b} can represent OH; OCF₃; O-methyl; O-ethyl; O—(CH₂)₂—O—CH₃; O—(CH₂)₂—OH; NH₂; NH-methyl; N(methyl)₂; NH-ethyl; N(ethyl)₂; or N(methyl)(ethyl);
   m represents 0, 1 or 2;
   Z represents C₁₋₄ alkyl, saturated or unsaturated, branched or unbranched, unsubstituted or mono- or polysubstituted with one or more substituents each selected independently of one another from the group consisting of F, Cl, Br, I, OH, ═O, O—C₁₋₄ alkyl, OCF₃, C(═O)—OH and CF₃; phenyl, naphthyl, furyl, pyridyl or thienyl, respectively unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₈ alkyl, SCF₃, benzyl and phenyl, wherein benzyl and phenyl can be respectively unsubstituted or mono- or polysubstituted with one or more substituents selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl and SCF₃; C₃₋₁₀ cycloalkyl or heterocyclyl, respectively saturated or unsaturated, unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, benzyl, phenyl and pyridyl, wherein benzyl, phenyl and pyridyl can be respectively unsubstituted or mono- or polysubstituted with one or more substituents selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl and SCF₃.

If m≠0, then the residues R^{13a} and R^{13b} can, taking account of the foregoing condition, both on the same carbon atom and on different carbon atoms, each independently of one another represent H; F; Cl; Br; I; NO₂; CF₃; CN; methyl; ethyl; n-propyl; isopropyl; n-butyl; sec.-butyl; tert.-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂₁ or N(methyl)(ethyl).

Particularly preferably, the residue

R¹ represents substructure (T1) in which
G represents C(═O), O, S, S(═O)₂, NH—C(═O) or NR¹⁴,
wherein R¹⁴ represents H; methyl; ethyl; n-propyl; isopropyl; n-butyl; sec.-butyl; tert.-butyl; S(═O)₂-methyl; S(═O)₂-ethyl;
   o represents 0 or 1;
   R^{13a} and R^{13b} each independently of one another represent H; F; Cl; Br; I; methyl; ethyl; n-propyl; isopropyl; n-butyl; sec.-butyl; tert.-butyl; OH; O-methyl; O-ethyl;
on the condition that if R^{13a} and R^{13b} are bound to the same carbon atom, only one of the substituents R^{13a} and R^{13b} can represent OH; O-methyl; O-ethyl;
   m represents 0, 1 or 2;
   Z represents C₁₋₄ alkyl, saturated or unsaturated, branched or unbranched, unsubstituted or mono- or polysubstituted with one or more substituents each selected independently of one another from the group consisting of F, Cl, Br, I, OH, O—C₁₋₄ alkyl, OCF₃, and CF₃;
C₃₋₁₀ cycloalkyl, saturated or unsaturated, unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, benzyl and phenyl, wherein benzyl and phenyl can be respectively unsubstituted or mono- or polysubstituted with one or more substituents selected independently of one another from the group consisting of F, Cl, Br, I, OH, O—C₁₋₄ alkyl, OCF₃, C₁₋₄ alkyl, CF₃, and SCF₃;
morpholinyl, thiomorpholinyl, piperidinyl, pyrrolidinyl, 4-methylpiperazinyl, piperazinyl, respectively unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, benzyl and phenyl, wherein benzyl and phenyl can be respectively unsubstituted or mono- or polysubstituted with one or more substituents selected independently of one another from the group consisting of F, Cl, Br, I, OH, O—C₁₋₄ alkyl, OCF₃, C₁₋₄ alkyl, CF₃ and SCF₃;
phenyl, naphthyl, pyridyl or thienyl, respectively unsubstituted or mono- or polysubstituted with one or more substituents each selected independently of one another from the group consisting of F, Cl, Br, I, CN, OH, C₁₋₄ alkyl, O—C₁₋₄ alkyl, OCF₃, C₁₋₄ alkyl, CF₃, SH, S—C₁₋₄ alkyl, SCF₃, benzyl and phenyl, wherein benzyl and phenyl can be respectively unsubstituted or mono- or polysubstituted with one or more substituents selected independently of one another from the group consisting of F, Cl, Br, I, OH, O—C₁₋₄ alkyl, OCF₃, C₁₋₄ alkyl, CF₃ and SCF₃.

If m≠0, then the residues R^{13a} and R^{13b} can, taking account of the foregoing condition, both on the same carbon atom and on different carbon atoms, each independently of one another represent H; F; Cl; Br; I; methyl; ethyl; n-propyl; isopropyl; n-butyl; sec.-butyl; tert.-butyl; OH; O-methyl; O-ethyl.

Most particularly preferably, the residue

R¹ represents substructure (T1) in which
G represents C(═O), O, S, S(═O)₂, NH—C(═O) or NR¹⁴,
wherein R¹⁴ represents H; methyl; ethyl; n-propyl; isopropyl; n-butyl; sec.-butyl; tert.-butyl; S(═O)₂-methyl;
   o represents 0 or 1;
   R^{13a} and R^{13b} each independently of one another represent H; methyl; ethyl; n-propyl; isopropyl; n-butyl; sec.-butyl; tert.-butyl;
   m represents 0, 1 or 2;
   Z represents C₁₋₄ alkyl, saturated or unsaturated, branched or unbranched, unsubstituted or mono- or polysubstituted with one or more substituents each selected independently of one another from the group consisting of F, Cl, Br, I, OH and O—C₁₋₄ alkyl;
C₃₋₁₀ cycloalkyl, saturated or unsaturated, respectively unsubstituted or mono- or polysubstituted with one or more substituents each selected independently of one another from the group consisting of F, Cl, Br, I, OH, O—C₁₋₄ alkyl and C₁₋₄ alkyl;
morpholinyl, piperidinyl, 4-methylpiperazinyl, piperazinyl, respectively unsubstituted or mono- or polysubstituted with one or more substituents each selected independently of one another from the group consisting of F, Cl, Br, I, OH, O—C₁₋₄ alkyl and C₁₋₄ alkyl;
phenyl or pyridyl, respectively unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, SH, S—C₁₋₄ alkyl and SCF₃.

If m≠0, then the residues R^{13a} and R^{13b} can, both on the same carbon atom and on different carbon atoms, each independently of one another represent H; methyl; ethyl; n-propyl; isopropyl; n-butyl; sec.-butyl; tert.-butyl.

In a further preferred embodiment of the compounds according to the invention of general formula (I), the residue
R² represents H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; C₁₋₁₀ alkyl, saturated or unsaturated, branched or unbranched, unsubstituted or mono- or polysubstituted with one or more substituents each selected independently of one another from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; C₃₋₁₀ cycloalkyl or heterocyclyl, respectively saturated or unsaturated, unsubstituted or mono- or polysubstituted with one or more substituents selected independently of one another from the group consisting of F, Cl, Br, I, OH, ═O, C₁₋₄ alkyl, O—C₁₋₄ alkyl, OCF₃, C(═O)—OH and CF₃; or C₃₋₁₀ cycloalkyl or heterocyclyl bridged via C₁₋₈ alkyl, respectively saturated or unsaturated, unsubstituted or mono- or polysubstituted with one or more substituents each selected independently of one another from the group consisting of F, Cl, Br, I, OH, ═O, C₁₋₄ alkyl, O—C₁₋₄ alkyl, OCF₃, C(═O)—OH and CF₃, wherein the alkyl chain can be respectively branched or unbranched, saturated or unsaturated, unsubstituted, mono- or polysubstituted with one or more substituents each selected independently of one another from the group consisting of F, Cl, Br, I, OH, ═O and O—C₁₋₄ alkyl; aryl or heteroaryl, respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₈ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or aryl or heteroaryl bridged via C₁₋₈ alkyl, respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₈ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH, wherein the alkyl chain can be respectively branched or unbranched, saturated or unsaturated, unsubstituted, mono- or polysubstituted with one or more substituents each selected independently of one another from the group consisting of F, Cl, Br, I, OH, ═O and O—C₁₋₄ alkyl.

Preferably, the residue
R² represents H; F; Cl; Br; I; CN; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; C₁₋₁₀ alkyl, saturated or unsaturated, branched or unbranched, unsubstituted or mono- or polysubstituted with one or more substituents each selected independently of one another from the group consisting of F, Cl, Br, I, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃, C₃₋₁₀ cycloalkyl, saturated or unsaturated, unsubstituted or mono- or polysubstituted with one or more substituents selected independently of one another from the group consisting of F, Cl, Br, I, OH, ═O, C₁₋₄ alkyl, O—C₁₋₄ alkyl, OCF₃ and CF₃; or C₃₋₁₀ cycloalkyl bridged via C₁₋₈ alkyl, saturated or unsaturated, unsubstituted or mono- or polysubstituted with one or more substituents selected independently of one another from the group consisting of F, Cl, Br, I, OH, ═O, C₁₋₄ alkyl, O—C₁₋₄ alkyl, OCF₃ and CF₃, wherein the alkyl chain can be respectively branched or unbranched, saturated or unsaturated, unsubstituted; aryl or heteroaryl, respectively unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₈ alkyl, SCF₃, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or aryl or heteroaryl bridged via C₁₋₈ alkyl, respectively unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₈ alkyl, SCF₃, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, alkyl, SCF₃ and S(═O)₂OH, wherein the alkyl chain can be respectively branched or unbranched, saturated or unsaturated, unsubstituted.

Particularly preferably,
R² represents H; F; Cl; Br; I; CN; C₁₋₁₀ alkyl, saturated or unsaturated, branched or unbranched, unsubstituted or mono- or polysubstituted with one or more substituents selected independently of one another from the group consisting of F, Cl, Br, I and OH; C₃₋₁₀ cycloalkyl, saturated or unsaturated, unsubstituted; or C₃₋₁₀ cycloalkyl bridged via C₁₋₄ alkyl, saturated or unsaturated, unsubstituted, wherein the alkyl chain can be branched or unbranched, saturated or unsaturated, unsubstituted; or phenyl, pyridyl, thienyl, respectively unsubstituted or mono- or polysubstituted with one or more substituents selected independently of one another from the group consisting of C₁₋₄ alkyl, O—C₁₋₄ alkyl, F, Cl, Br, I, CF₃, OCF₃, OH, SH and SCF₃; or phenyl, pyridyl or thienyl bridged via C₁₋₄ alkyl, respectively unsubstituted or mono- or polysubstituted with one or more substituents selected independently of one another from the group consisting of C₁₋₄ alkyl, O—C₁₋₄ alkyl, F, Cl, Br, I, CF₃, OCF₃, OH, SH and SCF₃, wherein the alkyl chain can be branched or unbranched, saturated or unsaturated, unsubstituted.

Also particularly preferably, the substituent
R² is selected from the group consisting of H; F; Cl; Br; I; CN; cyclopropyl; cyclobutyl; C₁₋₄ alkyl, saturated or unsaturated, branched or unbranched, unsubstituted, or mono- or polysubstituted with one or more substituents selected independently of one another from the group consisting of F, Cl, Br and phenyl, unsubstituted or mono- or polysubstituted with one or more substituents selected independently of one another from the group consisting of C₁₋₄ alkyl, O—C₁₋₄ alkyl, F, Cl, Br, I, CF₃ and OCF₃.

More particularly preferably, the substituent
R² represents H; F; Cl; Br; I; CF₃; CN; methyl; ethyl; n-propyl; isopropyl; n-butyl; sec.-butyl; tert.-butyl; cyclopropyl; cyclobutyl; phenyl, unsubstituted or mono- or polysubstituted with one or more substituents selected independently of one another from the group consisting of C₁₋₄ alkyl, O—C₁₋₄ alkyl, F, Cl, Br, I, CF₃ and OCF₃;

Especially particularly preferably, R² represents tert.-butyl, CF₃ or cyclopropyl.

In another preferred embodiment of the compounds according to the invention of general formula (I),
X represents N or CR³,
   wherein R³ represents H; C₁₋₁₀ alkyl, saturated or unsaturated, branched or unbranched, unsubstituted, mono- or polysubstituted with one or more substituents each selected independently of one another from the group consisting of F, Cl, Br, I and OH;

Preferably,
X represents N or CR³,
   wherein R³ represents H; C₁₋₁₀ alkyl, saturated or unsaturated, branched or unbranched, unsubstituted; or CF₃.

Particularly preferably,
X represents N or CR³,
   wherein R³ represents H; methyl; ethyl; n-propyl; isopropyl; n-butyl; sec.-butyl; tert.-butyl; or CF₃.

Most particularly preferably,
X represents N or CR³,
   wherein R³ represents H or CH₃, preferably represents H.

In a further preferred embodiment of the compounds according to the invention of general formula (I), the residue
R⁴ represents H or C₁₋₁₀ alkyl, saturated or unsaturated, branched or unbranched, unsubstituted or mono- or polysubstituted with one or more substituents each selected independently of one another from the group consisting of F, Cl, Br; I, OH and O—C₁₋₄ alkyl.

In a further preferred embodiment of the compounds according to the invention of general formula (I), the residue R⁴ represents H.

In a further preferred embodiment of the compounds according to the invention of general formula (I)
R^{5a} represents H; OH; C₁₋₁₀ alkyl, saturated or unsaturated, branched or unbranched, unsubstituted or mono- or polysubstituted with one or more substituents each selected independently of one another from the group consisting of F, Cl, Br; I, OH and O—C₁₋₄ alkyl;
R^{5b} represents H; C₁₋₁₀ alkyl, saturated or unsaturated, branched or unbranched, unsubstituted or mono- or polysubstituted with one or more substituents each selected independently of one another from the group consisting of F, Cl, Br; I, OH and O—C₁₋₄ alkyl; C₃₋₁₀ cycloalkyl or heterocyclyl, respectively saturated or unsaturated, unsubstituted or mono- or polysubstituted with one or more substituents each selected independently of one another from the group consisting of F, Cl, Br; I, OH, ═O and O—C₁₋₄ alkyl; or C₃₋₁₀ cycloalkyl or heterocyclyl bridged via C₁₋₈ alkyl, respectively saturated or unsaturated, unsubstituted or mono- or polysubstituted with one or more substituents each selected independently of one another from the group consisting of F, Cl, Br; I, OH, ═O and O—C₁₋₄ alkyl, wherein the alkyl chain can be respectively branched or unbranched, saturated or unsaturated, unsubstituted, mono- or polysubstituted with one or more substituents each selected independently of one another from the group consisting of F, Cl, Br; I, OH, ═O and O—C₁₋₄ alkyl; or aryl, heteroaryl, respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl; or aryl or heteroaryl bridged via C₁₋₈ alkyl, respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl, wherein the alkyl chain can be respectively branched or unbranched, saturated or unsaturated, unsubstituted, mono- or polysubstituted with one or more substituents each selected independently of one another from the group consisting of F, Cl, Br; I, OH, ═O and O—C₁₋₄ alkyl;
   or R^{5a} and R^{5b} form together with the carbon atom connecting them a C₃₋₁₀ cycloalkyl or a heterocyclyl, respectively saturated or unsaturated, unsubstituted or mono- or polysubstituted with one or more substituents each selected independently of one another from the group consisting of F, Cl, Br; I, OH, ═O and O—C₁₋₄ alkyl.

Preferably
R^{5a} represents H; or C₁₋₁₀ alkyl, saturated or unsaturated, branched or unbranched, unsubstituted;
R^{5b} represents H; C₁₋₁₀ alkyl, saturated or unsaturated, branched or unbranched, unsubstituted or mono- or polysubstituted with one or more substituents each selected independently of one another from the group consisting of F, Cl, Br, I, OH and O—C₁₋₄ alkyl; C₃₋₁₀ cycloalkyl, saturated or unsaturated, unsubstituted or mono- or polysubstituted with one or more substituents each selected independently of one another from the group consisting of F, Cl, Br, I and C₁₋₄ alkyl; or C₃₋₁₀ cycloalkyl bridged via C₁₋₄ alkyl, saturated or unsaturated, unsubstituted or mono- or polysubstituted with one or more substituents each selected independently of one another from the group consisting of F, Cl, Br, I and C₁₋₄ alkyl, wherein the alkyl chain can be respectively branched or unbranched, saturated or unsaturated, unsubstituted; or phenyl or pyridyl, respectively unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl, SCF₃ and NH—S(═O)₂—C₁₋₄ alkyl; or phenyl or pyridyl bridged via C₁₋₄ alkyl, respectively unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl, SCF₃ and NH—S(═O)₂—C₁₋₄ alkyl, wherein the alkyl chain can be respectively branched or unbranched, saturated or unsaturated, unsubstituted,
   or R^{5a} and R^{5b} form together with the carbon atom connecting them a C₃₋₁₀ cycloalkyl or a heterocyclyl, respectively saturated or unsaturated, unsubstituted or mono- or polysubstituted with one or more substituents each selected independently of one another from the group consisting of F, Cl, Br; I, OH, ═O and O—C₁₋₄ alkyl.

Particularly preferably,

R^{5a} represents H if A or CH₃, preferably H, represents N;
or R^{5a} represents H or CH₃, preferably H, if A represents CR^{5b},
wherein R^{5b} represents H; or C₁₋₄ alkyl, saturated or unsaturated, branched or unbranched, unsubstituted; C₃₋₁₀ cycloalkyl, saturated or unsaturated, unsubstituted; or phenyl or benzyl, in each case unsubstituted or mono- or polysubstituted with one or more substituents each selected independently of one another from the group consisting of F, Cl, Br, I, CF₃, O—C₁₋₄ alkyl, OCF₃ and C₁₋₄ alkyl,
   or R^{5a} and R^{5b} form together with the carbon atom connecting them a C₃₋₁₀ cycloalkyl, saturated or unsaturated, unsubstituted or mono- or polysubstituted with one or more substituents each selected independently of one another from the group consisting of F, Cl, Br, I, OH, ═O and O—C₁₋₄ alkyl.

Most particularly preferably, the residue

R^{5a} represents H;
R^{5b} represents H; or C₁₋₄ alkyl, saturated or unsaturated, branched or unbranched, unsubstituted; cyclohexyl, unsubstituted; or phenyl or benzyl, in each case unsubstituted or mono- or polysubstituted with one or more substituents each selected independently of one another from the group consisting of F, Cl, Br, I, O—C₁₋₄ alkyl, CF₃, OCF₃ and C₁₋₄ alkyl,
or R^{5a} and R^{5b} form together with the carbon atom connecting them a C₃₋₁₀ cycloalkyl, saturated or unsaturated, unsubstituted.

In a further preferred embodiment of the compounds according to the invention Y represents an oxygen atom (O).

In a further preferred embodiment of the compounds according to the invention of general formula (I) the partial structure (T2)

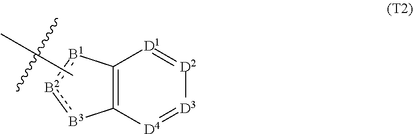

is selected from the following group consisting of

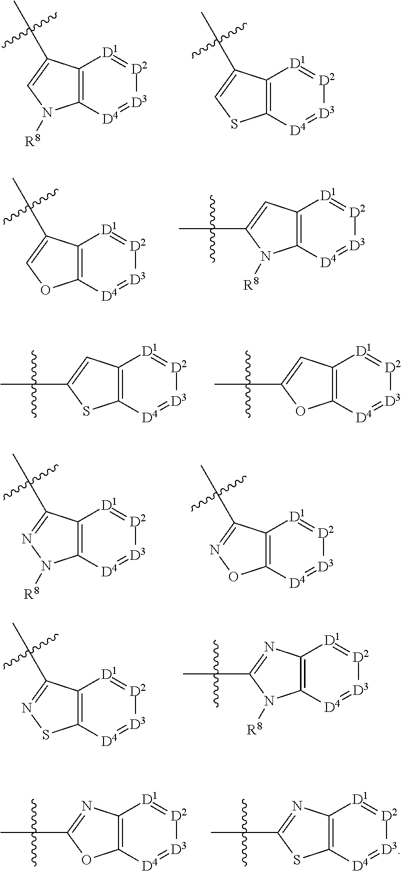

In another preferred embodiment of the compounds according to the invention of general formula (I) the partial structure (T2)

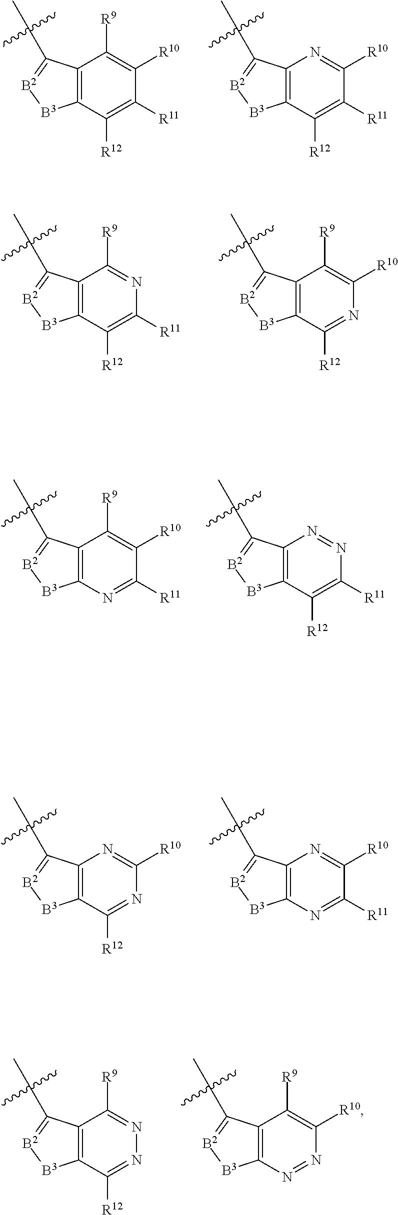

wherein in each case independently
B² represents C or N, and
B³ represents NR⁸, O or S;
or that the partial structure (T2) is selected from the following group consisting of

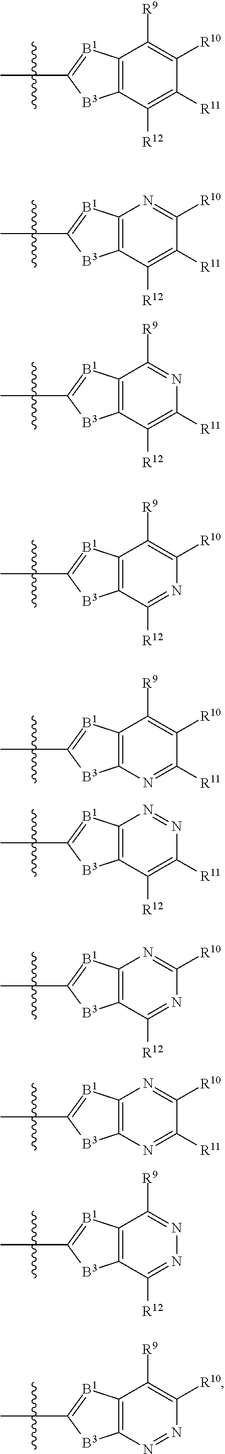

wherein in each case independently
B¹ represents C or N, and
B³ represents NR⁸, O or S.

In another preferred embodiment of the compounds according to the invention of general formula (I) the partial structure (T2) is selected from the following group consisting of

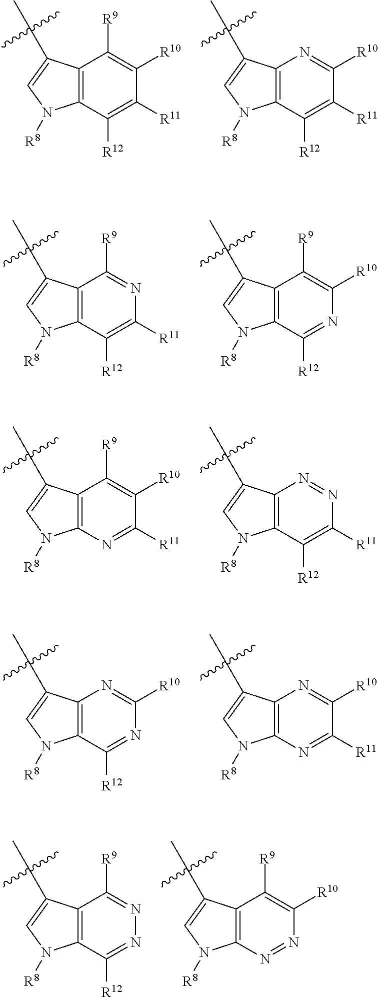

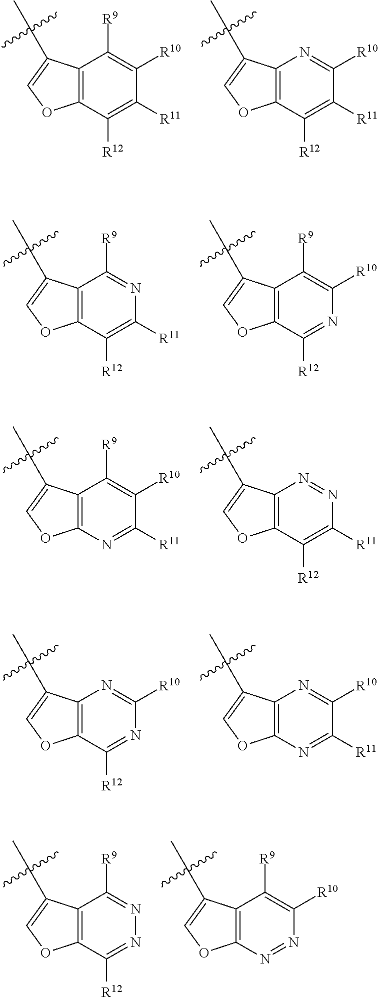

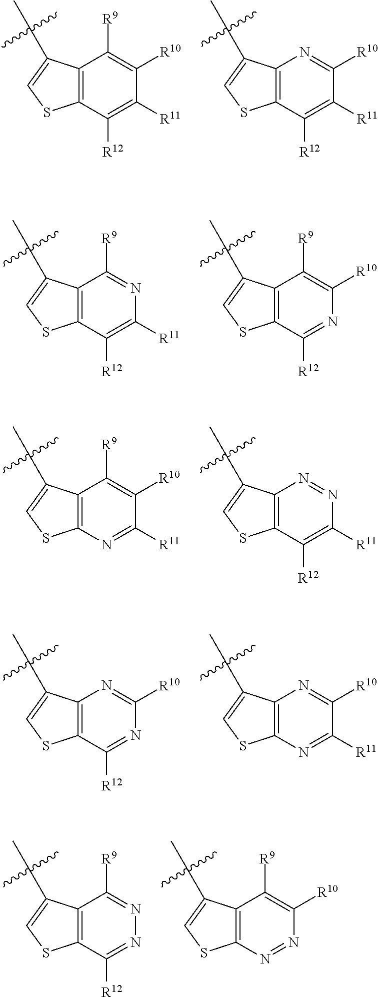

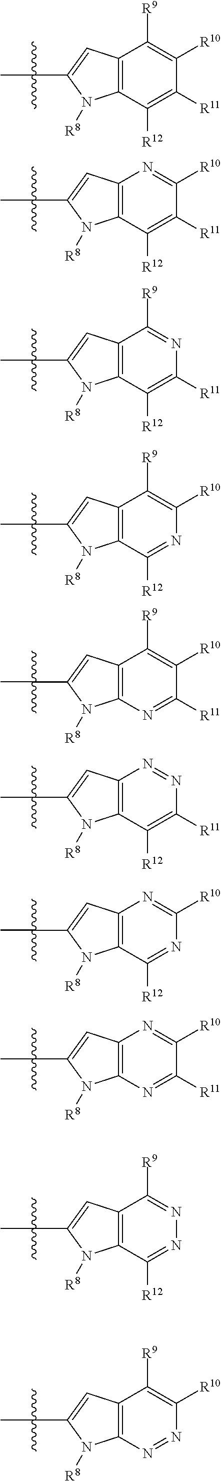

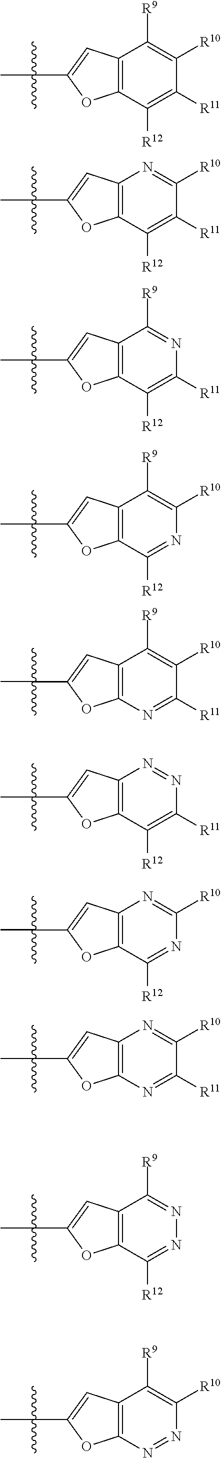

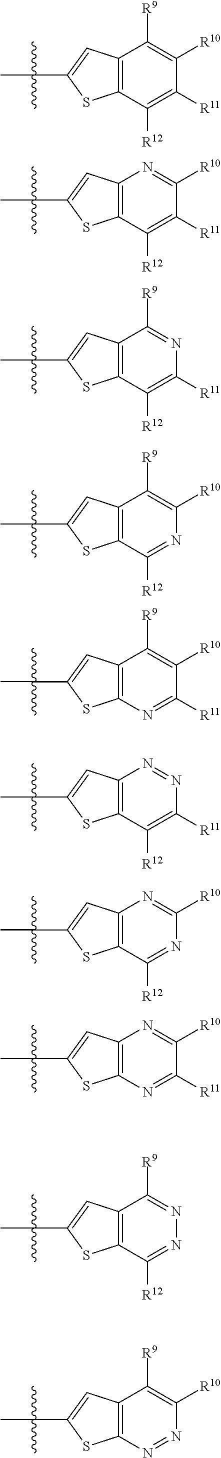

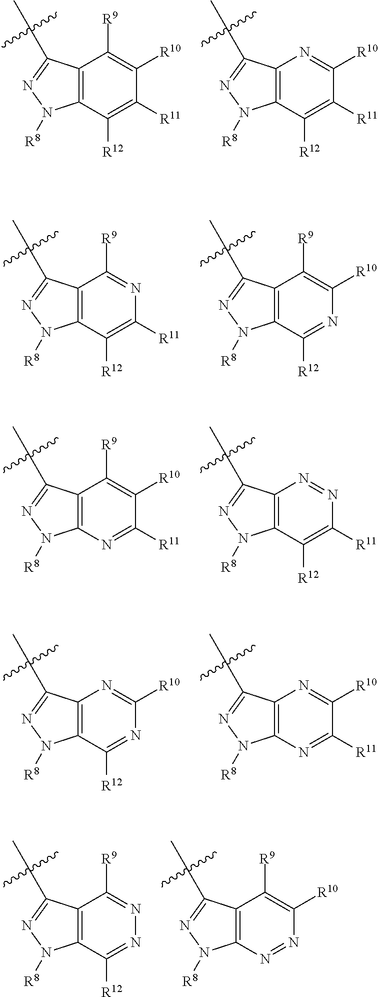

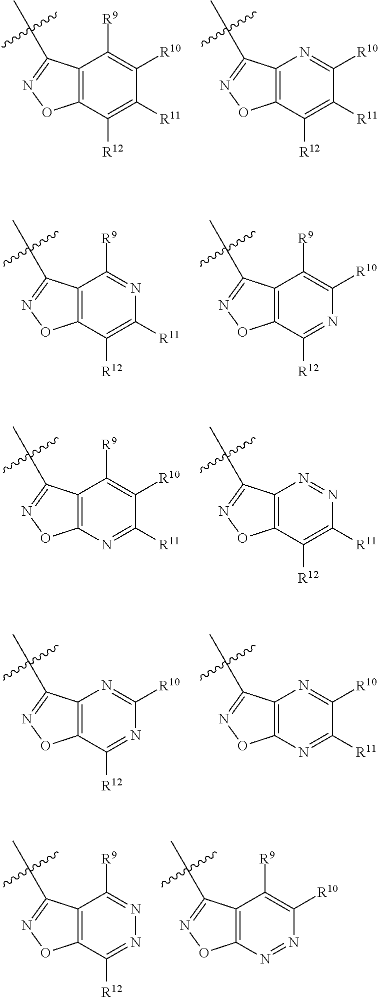

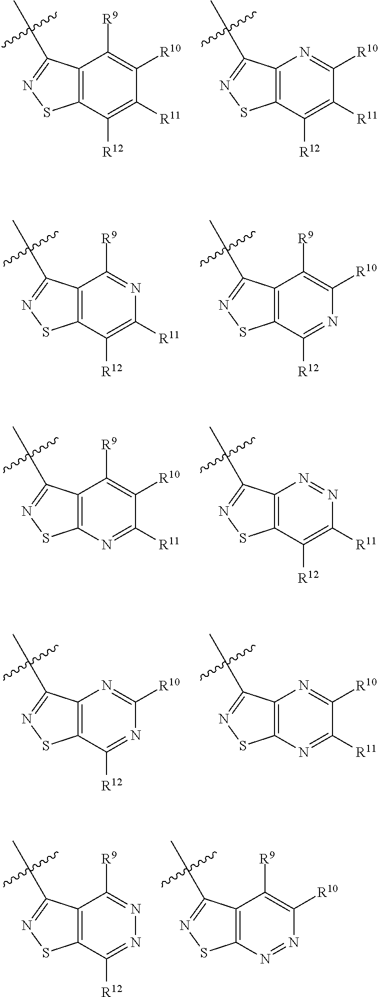

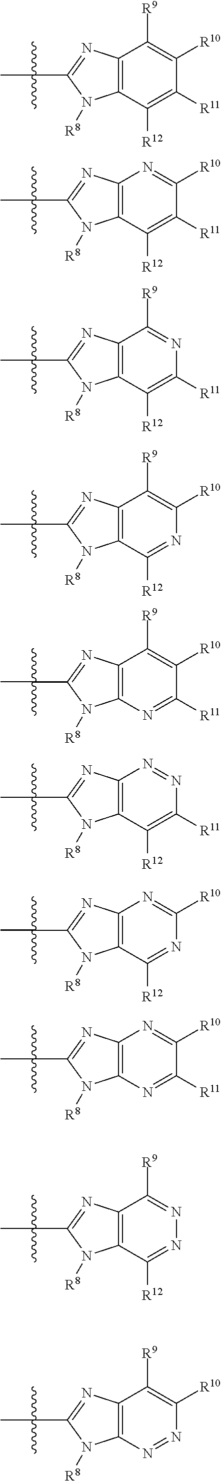

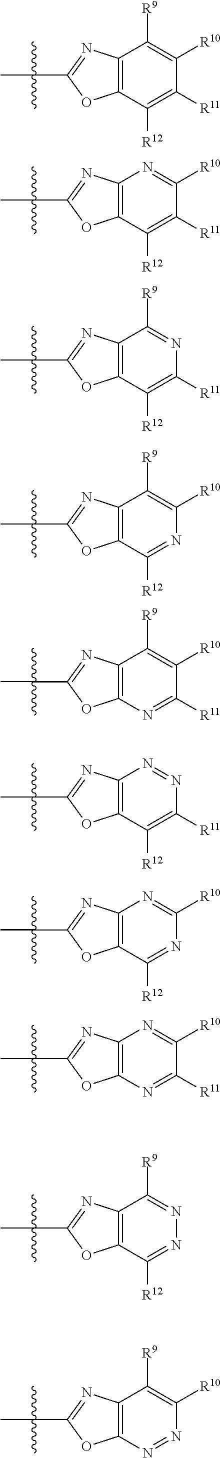

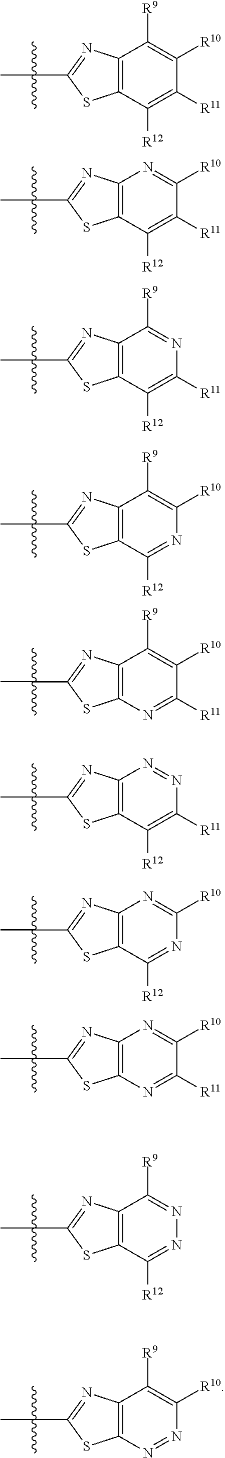

In another preferred embodiment of the compounds according to the invention of general formula (I) R⁰, R⁷ and R⁸ each independently of one another represent H, methyl or ethyl.

In another preferred embodiment of the compounds according to the invention of general formula (I) R⁹, R¹⁰, R¹¹ and R¹² are each selected independently of one another from the group consisting of H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C₁₋₁₀ alkyl, C₁₋₁₀ alkyl-O—C₁₋₁₀ alkyl, C(═O)—NH—C₁₋₁₀ alkyl, O—C₁₋₁₀ alkyl, NH(C₁₋₁₀ alkyl), N(C₁₋₁₀ alkyl)₂, NH—C(═O)—C₁₋₁₀ alkyl, N(C₁₋₁₀ alkyl)-C(═O)—C₁₋₁₀ alkyl, NH—S(═O)₂—C₁₋₁₀ alkyl, S—C₁₋₁₀ alkyl, SO₂—C₁₋₁₀ alkyl, SO₂—NH(C₁₋₁₀ alkyl), SO₂—N(C₁₋₁₀ alkyl)₂, in which C₁₋₁₀ alkyl can be respectively saturated or unsaturated, branched or unbranched, unsubstituted or mono- or polysubstituted with one or more substituents selected independently of one another from the group consisting of F, Cl, Br, I, NO₂, CN, OH, O—C₁₋₄ alkyl, OCF₃, CF₃, NH₂, NH(C₁₋₄ alkyl), N(C₁₋₄ alkyl)₂, NH—S(═O)₂—C₁₋₄ alkyl, N(C₁₋₄ alkyl)-S(═O)₂—C₁₋₄ alkyl, SH, S—C₁₋₄ alkyl, S(═O)₂—C₁₋₄ alkyl and SCF₃;

C₃₋₁₀ cycloalkyl, heterocyclyl or C₃₋₁₀ cycloalkyl or heterocyclyl bridged via C₁₋₈ alkyl, respectively saturated or unsaturated, unsubstituted or mono- or polysubstituted with one or more substituents selected independently of one another from the group consisting of F, Cl, Br, I, NO₂, CN, OH, O—C₁₋₄ alkyl, OCF₃, CF₃, C₁₋₄ alkyl, NH₂, NH(C₁₋₄ alkyl), N(C₁₋₄ alkyl)₂, NH—S(═O)₂—C₁₋₄ alkyl, N(C₁₋₄ alkyl)-S(═O)₂—C₁₋₄ alkyl, SH, S—C₁₋₄ alkyl, S(═O)₂—C₁₋₄ alkyl and SCF₃, and wherein if appropriate the alkyl chain can be respectively branched or unbranched, saturated or unsaturated, unsubstituted, mono- or polysubstituted with one or more substituents each selected independently of one another from the group consisting of F, Cl, Br; I, OH and O—C₁₋₄ alkyl;
aryl, heteroaryl, C(═O)—NH-aryl, C(═O)—NH-heteroaryl, NH—C(═O)-aryl, NH(C═O)-heteroaryl, NH(aryl), NH(heteroaryl), N(aryl)₂, N(heteroaryl)₂ or aryl or heteroaryl bridged via C₁₋₄ alkyl, respectively unsubstituted or mono- or polysubstituted with one or more substituents selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl and SCF₃, and wherein if appropriate the alkyl chain can be respectively branched or unbranched, saturated or unsaturated, unsubstituted, mono- or polysubstituted with one or more substituents each selected independently of one another from the group consisting of F, Cl, Br; I, OH and O—C₁₋₄ alkyl.

In yet another preferred embodiment of the compounds according to the invention of general formula (I) R⁹, R¹⁰, R¹¹ and R¹² are each selected independently of one another from the group consisting of H; F; Cl; Br; I; CN; NO₂; CF₃; OH; OCF₃; SH; SCF₃; NH₂; C(═O)—NH₂; C₁₋₄ alkyl, C₁₋₄ alkyl-O—C₁₋₄ alkyl, C(═O)—NH—C₁₋₄ alkyl, O—C₁₋₄ alkyl, NH(C₁₋₄ alkyl), N(C₁₋₄ alkyl)₂, NH—C(═O)—C₁₋₄ alkyl, NH—S(═O)₂—C₁₋₄ alkyl, S—C₁₋₄ alkyl, SO₂—C₁₋₄ alkyl, SO₂—NH(C₁₋₄ alkyl), SO₂—N(C₁₋₄ alkyl)₂, in which C₁₋₄ alkyl can be respectively saturated or unsaturated, branched or unbranched, unsubstituted or mono- or polysubstituted with one or more substituents selected independently of one another from the group consisting of F, Cl, Br, I, OH, O—C₁₋₄ alkyl, OCF₃, CF₃, NH—S(═O)₂—C₁₋₄ alkyl, SH, S—C₁₋₄ alkyl, S(═O)₂—C₁₋₄ alkyl and SCF₃; C₃₋₁₀ cycloalkyl, heterocyclyl or C₃₋₁₀ cycloalkyl or heterocyclyl bridged via C₁₋₈ alkyl, respectively saturated or unsaturated, unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, CF₃, NH₂, NH(C₁₋₄ alkyl), N(C₁₋₄ alkyl)₂, NH—S(═O)₂—C₁₋₄ alkyl, N(C₁₋₄ alkyl)-S(═O)₂—C₁₋₄ alkyl, SH, S—C₁₋₄ alkyl, S(═O)₂—C₁₋₄ alkyl and SCF₃, and wherein if appropriate the alkyl chain can be respectively branched or unbranched, saturated or unsaturated, unsubstituted, mono- or polysubstituted with one or more substituents each selected independently of one another from the group consisting of F, Cl, Br; I, OH and O—C₁₋₄ alkyl; phenyl, pyridyl, furyl, thienyl, C(═O)—NH-phenyl, NH—C(═O)-phenyl, NH(phenyl), C(═O)—NH-pyridyl, NH—C(═O)-pyridyl, NH(pyridyl) or phenyl or pyridyl bridged via C₁₋₈ alkyl, wherein phenyl, pyridyl, furyl or thienyl are respectively unsubstituted or mono- or polysubstituted with one or more substituents selected independently of one another from the group consisting of F, Cl, Br, I, CN, OH, O—C₁₋₄ alkyl, OCF₃, C₁₋₄ alkyl, CF₃, SH, S—C₁₋₄ alkyl and SCF₃, and wherein if appropriate the alkyl chain can be respectively branched or unbranched, saturated or unsaturated, unsubstituted, mono- or polysubstituted with one or more substituents each selected independently of one another from the group consisting of F, Cl, Br; I, OH and O—C₁₋₄ alkyl.

In another preferred embodiment of the compounds according to the invention of general formula (I) R⁹, R¹⁰, R¹¹ and R¹² are each selected independently of one another from the group consisting of H; F; Cl; Br; I; CF₃; OCF₃; SCF₃; C₁₋₄ alkyl, O—C₁₋₄ alkyl and NH—S(═O)₂—C₁₋₄ alkyl, in which C₁₋₄ alkyl can be respectively saturated or unsaturated, branched or unbranched, unsubstituted.

In yet another preferred embodiment the present invention relates to compounds of formula (I′)

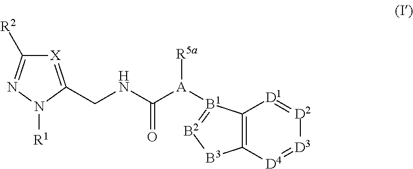

wherein R¹ represents the partial structure (T1)

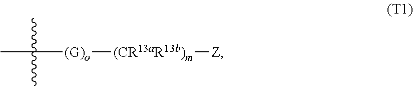

in which
G represents C(═O), O, S, S(═O)₂, NH—C(═O) or NR¹⁴,
wherein R¹⁴ represents H; methyl; ethyl; n-propyl; isopropyl; n-butyl; sec.-butyl; tert.-butyl; S(═O)₂-methyl; S(═O)₂-ethyl;
o represents 0 or 1;
R^{13a} and R^{13b} each independently of one another represent H; F; Cl; Br; I; methyl; ethyl; n-propyl; isopropyl; n-butyl; sec.-butyl; tert.-butyl; OH; O-methyl; O-ethyl;
on the condition that if R^{13a} and R^{13b} are bound to the same carbon atom, only one of the substituents R^{13a} and R^{13b} can represent OH; O-methyl; O-ethyl;
m represents 0, 1 or 2;
Z represents C₁₋₄ alkyl, saturated or unsaturated, branched or unbranched, unsubstituted or mono- or polysubstituted with one or more substituents each selected independently of one another from the group consisting of F, Cl, Br, I, OH, O—C₁₋₄ alkyl, OCF₃, and CF₃;
C₃₋₁₀ cycloalkyl, saturated or unsaturated, unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, benzyl and phenyl, wherein benzyl and phenyl can be respectively unsubstituted or mono- or polysubstituted with one or more substituents selected independently of one another from the group consisting of F, Cl, Br, I, OH, O—C₁₋₄ alkyl, OCF₃, C₁₋₄ alkyl, CF₃, and SCF₃;
morpholinyl, thiomorpholinyl, piperidinyl, pyrrolidinyl, 4-methylpiperazinyl, piperazinyl, respectively unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, benzyl and phenyl, wherein benzyl and phenyl can be respectively unsubstituted or mono- or polysubstituted with one or more substituents selected independently of one another from the group consisting of F, Cl, Br, I, OH, O—C₁₋₄ alkyl, OCF₃, C₁₋₄ alkyl, CF₃ and SCF₃;
phenyl, naphthyl, pyridyl or thienyl, respectively unsubstituted or mono- or polysubstituted with one or more substituents each selected independently of one another from the group consisting of F, Cl, Br, I, CN, OH, C₁₋₄ alkyl, O—C₁₋₄ alkyl, OCF₃, C₁₋₄ alkyl, CF₃, SH, S—C₁₋₄ alkyl, SCF₃, benzyl and phenyl, wherein benzyl and phenyl can be respectively unsubstituted or mono- or polysubstituted with one or more substituents selected independently of one another from the group consisting of F, Cl, Br, I, OH, O—C₁₋₄ alkyl, OCF₃, C₁₋₄ alkyl, CF₃ and SCF₃.
R² represents tert-butyl, CF₃ or cyclopropyl;
X represents CR³ or N,
wherein R³ represents H or C₁₋₄ alkyl, saturated, branched or unbranched, unsubstituted;
A represents N or CR^{5b};
R^{5a} represents H;
R^{5b} represents H; or C₁₋₄ alkyl, saturated or unsaturated, branched or unbranched, unsubstituted; cyclohexyl, unsubstituted; or phenyl or benzyl, in each case unsubstituted or mono- or polysubstituted with one or more substituents each selected independently of one another from the group consisting of F, Cl, Br, I, O—C₁₋₄ alkyl, CF₃, OCF₃ and C₁₋₄ alkyl,
or R^{5a} and R^{5b} form together with the carbon atom connecting them a C₃₋₁₀ cycloalkyl, saturated or unsaturated, unsubstituted;
B¹ represents C, CH, N, NR⁶, O or S;
B² represents C, CH, N, NR⁷, O or S;
B³ represents C, CH, N, NR⁸, O or S;
wherein 1 or 2 of the variables B¹, B² and B³ represent one of the afore mentioned heteroatoms or heteroatom groups;
D¹ represents N or CR⁹;
D² represents N or CR¹⁰;
D³ represents N or CR¹¹;
D⁴ represents N or CR¹²;
wherein 0, 1 or 2 of the variables D¹, D², D³ and D⁴ represent N;
R⁶, R⁷ and R⁸ each independently of one another represent H or C₁₋₄ alkyl, saturated, branched or unbranched, unsubstituted or mono- or polysubstituted; and
R⁹, R¹⁰, R¹¹ and R¹² are each selected independently of one another from the group consisting of H; F; Cl; Br; I; CF₃; OCF₃; SCF₃; C₁₋₄ alkyl, O—C₁₋₄ alkyl and NH—S(═O)₂—C₁₋₄ alkyl, in which C₁₋₄ alkyl can be respectively saturated or unsaturated, branched or unbranched, unsubstituted;
in the form of the free compounds; the tautomers; the N-oxides; the racemate; the enantiomers, diastereomers, mixtures of the enantiomers or diastereomers or of an individual enantiomer or diastereomer; or in the form of the salts of physiologically compatible acids or bases.

In another preferred embodiment the present invention relates to compounds of general structures C1-C7

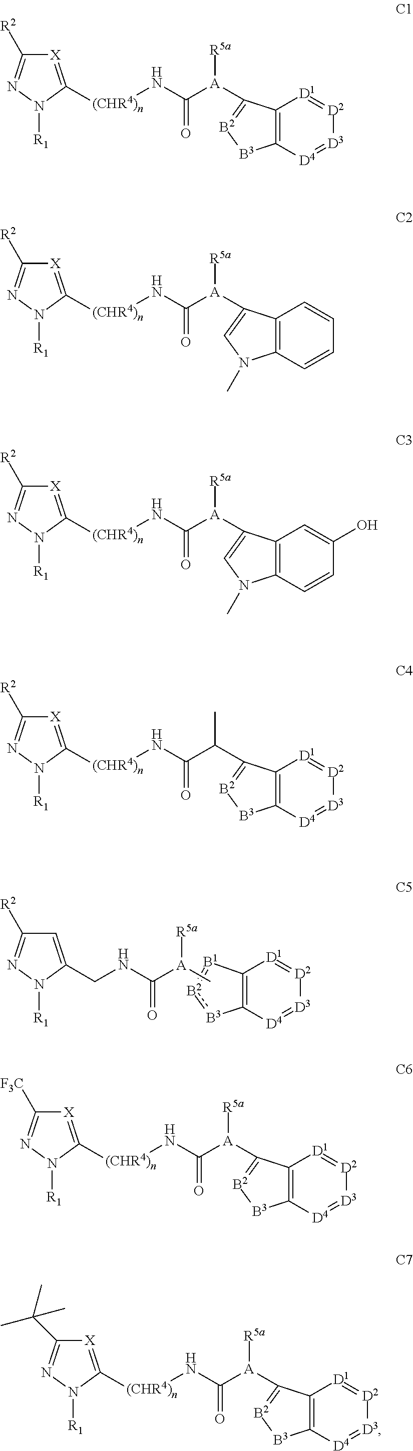

wherein the respective variables, substituents and indices have one of the meanings as described herein.

In yet another preferred embodiment the present invention relates to substituted compounds of formula (I) selected from the group consisting of:
[1] 2-(1-methyl-1H-indol-3-yl)-N-((1-phenyl-3-(trifluoromethyl)-1H-pyrazol-5-yl)methyl)propanamide,
[2] N-((3-tert-butyl-1-(3-chlorophenyl)-1H-pyrazol-5-yl)methyl)-2-(1-methyl-1H-indol-3-yl)propanamide,
[3] N-((1-(3-methoxyphenyl)-3-(trifluoromethyl)-1H-pyrazol-5-yl)methyl)-2-(1-methyl-1H-indol-3-yl)propanamide,
[4] N-((1-cyclopentyl-3-(trifluoromethyl)-1H-pyrazol-5-yl)methyl)-2-(5-hydroxy-1-methyl-1H-indol-3-yl)propanamide,
[5] 2-(5-hydroxy-1-methyl-1H-indol-3-yl)-N-((1-m-tolyl-3-(trifluoromethyl)-1H-pyrazol-5-yl)methyl)propanamide,
[6] N-((3-tert-butyl-1-(pyridin-2-yl)-1H-pyrazol-5-yl)methyl)-2-(5-hydroxy-1-methyl-1H-indol-3-yl)propanamide,
[7] N-((1-(3-chlorophenyl)-4-methyl-3-(trifluoromethyl)-1H-pyrazol-5-yl)methyl)-2-(5-hydroxy-1-methyl-1H-indol-3-yl)propanamide,
[8] N-((1-(3-chlorophenyl)-3-(trifluoromethyl)-1H-pyrazol-5-yl)methyl)-2-(1-methyl-1H-indol-3-yl)propanamide,
[9] N-((1-(3-chlorophenyl)-3-(trifluoromethyl)-1H-pyrazol-5-yl)methyl)-2-(5-hydroxy-1-methyl-1H-indol-3-yl)acetamide,
[10] N-((1-(3-chlorophenyl)-3-(trifluoromethyl)-1H-pyrazol-5-yl)methyl)-2-(5-hydroxy-1-methyl-1H-indol-3-yl)propanamide,
[11] N-((3-tert-butyl-1-(3-chloro-4-fluorophenyl)-1H-pyrazol-5-yl)methyl)-2-(5-hydroxy-1-methyl-1H-indol-3-yl)propanamide,
[12] N-((1-(3-chlorophenyl)-3-cyclopropyl-1H-pyrazol-5-yl)methyl)-2-(5-hydroxy-1-methyl-1H-indol-3-yl)propanamide,
[13] N-((3-tert-butyl-1-(3-chlorophenyl)-1H-pyrazol-5-yl)methyl)-2-(5-methoxy-1-methyl-1H-indol-3-yl)acetamide
[14] N-((3-tert-butyl-1-(3-chlorophenyl)-1H-pyrazol-5-yl)methyl)-2-(5-methoxy-1-methyl-1H-indol-3-yl)propanamide,
[15] N-((1-(cyclopropylmethyl)-3-(trifluoromethyl)-1H-pyrazol-5-yl)methyl)-2-(1-methyl-1H-indol-3-yl)propanamide,
[16] 1-((1-(3-chlorophenyl)-3-(trifluoromethyl)-1H-pyrazol-5-yl)methyl)-3-(1H-indazol-3-yl)urea,
[17] 1-((1-(3-chlorophenyl)-3-(trifluoromethyl)-1H-pyrazol-5-yl)methyl)-3-(1-methyl-1H-pyrazolo[3,4-b]pyridin-3-yl)urea and
[18] N-((3-tert-butyl-1-(3-chlorophenyl)-1H-1,2,4-triazol-5-yl)methyl)-2-(5-hydroxy-1-methyl-1H-indol-3-yl)acetamide
[19] N-((3-tert-butyl-1-methyl-1H-pyrazol-5-yl)methyl)-2-(5-hydroxy-1-methyl-1H-indol-3-yl)propanamide
[20] N-((3-tert-butyl-1-hexyl-1H-pyrazol-5-yl)methyl)-2-(5-hydroxy-1-methyl-1H-indol-3-yl)propanamide
[21] N-((1-cyclohexyl-3-(trifluoromethyl)-1H-pyrazol-5-yl)methyl)-2-(5-hydroxy-1-methyl-1H-indol-3-yl)propanamide
[22] 2-(5-hydroxy-1-methyl-1H-indol-3-yl)-N-((1-(tetrahydro-2H-pyran-4-yl)-3-(trifluoromethyl)-1H-pyrazol-5-yl)methyl)propanamide
[23] 2-(5-hydroxy-1-methyl-1H-indol-3-yl)-N-((1-(oxetan-3-yl)-3-(trifluoromethyl)-1H-pyrazol-5-yl)methyl)propanamide
[24] N-((3-tert-butyl-1-(3-fluorophenyl)-1H-pyrazol-5-yl)methyl)-2-(5-hydroxy-1-methyl-1H-indol-3-yl)propanamide
[25] N-((3-tert-butyl-1-(4-methoxyphenyl)-1H-pyrazol-5-yl)methyl)-2-(5-hydroxy-1-methyl-1H-indol-3-yl)propanamide
[26] N-((3-tert-butyl-1-(3-chloro-4-fluorophenyl)-1H-pyrazol-5-yl)methyl)-2-(5-hydroxy-1-methyl-1H-indol-3-yl)propanamide
[27] 2-(5-hydroxy-1-methyl-1H-indol-3-yl)-N-((1-(4-methoxybenzyl)-3-(trifluoromethyl)-1H-pyrazol-5-yl)methyl)propanamide
[28] 2-(5-hydroxy-1-methyl-1H-indol-3-yl)-N-((1-phenyl-3-(trifluoromethyl)-1H-pyrazol-5-yl)methyl)propanamide
[29] N-((1-(4-fluorophenyl)-3-(trifluoromethyl)-1H-pyrazol-5-yl)methyl)-2-(5-hydroxy-1-methyl-1H-indol-3-yl)propanamide
[30] 2-(5-hydroxy-1-methyl-1H-indol-3-yl)-N-((1-(3-hydroxyphenyl)-3-(trifluoromethyl)-1H-pyrazol-5-yl)methyl)propanamide
[31] 2-(5-hydroxy-1-methyl-1H-indol-3-yl)-N-((1-(3-isopropylphenyl)-3-(trifluoromethyl)-1H-pyrazol-5-yl)methyl)propanamide
[32] 2-(5-hydroxy-1-methyl-1H-indol-3-yl)-N-((1-(pyridin-3-yl)-3-(trifluoromethyl)-1H-pyrazol-5-yl)methyl)propanamide
[33] 2-(5-hydroxy-1-methyl-1H-indol-3-yl)-N-((1-(pyrimidin-2-yl)-3-(trifluoromethyl)-1H-pyrazol-5-yl)methyl)propanamide
[34] 1-((1-(3-chlorophenyl)-3-(trifluoromethyl)-1H-pyrazol-5-yl)methyl)-3-(1-methyl-1H-pyrrolo[2,3-c]pyridin-3-yl)urea
[35] N-((1-(3-chlorophenyl)-3-(trifluoromethyl)-1H-pyrazol-5-yl)methyl)-2-(1,5-dimethyl-1H-indol-3-yl)propanamide
[36] 1-((1-(3-chlorophenyl)-3-(trifluoromethyl)-1H-pyrazol-5-yl)methyl)-3-(5-fluoro-1-methyl-1H-indol-3-yl)urea
[37] 1-((1-(3-chlorophenyl)-3-(trifluoromethyl)-1H-pyrazol-5-yl)methyl)-3-(7-methyl-7 H-pyrrolo[2,3-d]pyrimidin-5-yl)urea
[38] 1-((1-(3-chlorophenyl)-3-(trifluoromethyl)-1H-pyrazol-5-yl)methyl)-3-(5-(dimethylamino)-1-methyl-1H-indol-3-yl)urea
[40] 1-((1-(3-chlorophenyl)-3-(trifluoromethyl)-1H-pyrazol-5-yl)methyl)-3-(5-hydroxybenzo[d]oxazol-2-yl)urea
[41] 1-((1-(3-chlorophenyl)-3-(trifluoromethyl)-1H-pyrazol-5-yl)methyl)-3-(6-hydroxybenzo[d]oxazol-2-yl)urea
[42] 1-((1-(3-chlorophenyl)-3-(trifluoromethyl)-1H-pyrazol-5-yl)methyl)-3-(4-hydroxybenzo[d]oxazol-2-yl)urea
[43] 1-(1H-benzo[d]imidazol-2-yl)-3-((1-(3-chlorophenyl)-3-(trifluoromethyl)-1H-pyrazol-5-yl)methyl)urea
[44] 1-(1H-benzo[d]imidazol-2-yl)-3-((1-(3-chlorophenyl)-3-(trifluoromethyl)-1H-pyrazol-5-yl)methyl)urea
[45] 1-((1-(3-chlorophenyl)-3-(trifluoromethyl)-1H-pyrazol-5-yl)methyl)-3-(1-methyl-1H-benzo[d]imidazol-2-yl)urea
[46] 1-(6-chloro-1H-benzo[d]imidazol-2-yl)-3-((1-(3-chlorophenyl)-3-(trifluoromethyl)-1H-pyrazol-5-yl)methyl)urea
[47] 1-(5-chlorobenzo[d]oxazol-2-yl)-3-((1-(3-chlorophenyl)-3-(trifluoromethyl)-1H-pyrazol-5-yl)methyl)urea
[48] 1-((1-(3-chlorophenyl)-3-(trifluoromethyl)-1H-pyrazol-5-yl)methyl)-3-(6-methoxybenzo[d]thiazol-2-yl)urea
[49] 1-((1-(3-chlorophenyl)-3-(trifluoromethyl)-1H-pyrazol-5-yl)methyl)-3-(6-(methylsulfonyl)benzo[d]thiazol-2-yl)urea
   respectively in the form of the free compounds; the racemate; the enantiomers, diastereomers, mixtures of the enantiomers or diastereomers or of an individual enantiomer or diastereomer; or in the form of the salts of physiologically compatible acids or bases.

Furthermore, preference may be given to compounds according to the invention of general formula (I) that cause a 50 percent displacement of capsaicin, which is present at a concentration of 100 nM, in a FLIPR assay with CHO K1 cells which were transfected with the human VR1 gene at a concentration of less than 2,000 nM, preferably less than 1,000 nM, particularly preferably less than 300 nM, most particularly preferably less than 100 nM, even more preferably less than 75 nM, additionally preferably less than 50 nM, most preferably less than 10 nM.

In the process, the Ca²⁺ influx is quantified in the FLIPR assay with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA), as described hereinafter.

The present invention further relates to a process for preparing compounds of the above-indicated general formula (I), according to which at least one compound of general formula (II),

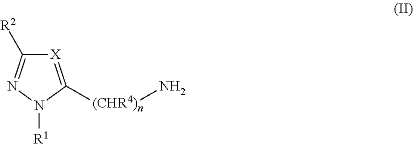

in which X, R¹, R², R⁴ and n have one of the foregoing meanings, is reacted in a reaction medium, if appropriate in the presence of at least one suitable coupling reagent, if appropriate in the presence of at least one base, with a compound of general formula (III) or (IV),

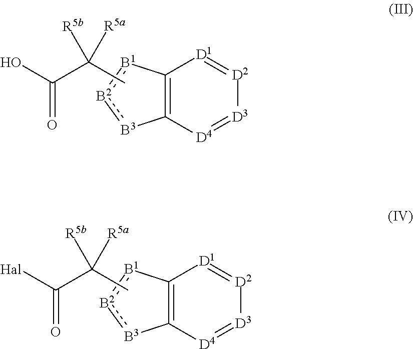

in which Hal represents a halogen, preferably Br or Cl, and R^{5a}, R^{5b}, B¹, B², B³, D¹, D², D³ and D⁴ each have one of the foregoing meanings, in a reaction medium, if appropriate in the presence of at least one suitable coupling reagent, if appropriate in the presence of at least one base, to form a compound of general formula (I) in which A represents CR^{5b} and the other variables, substituents and indices have one of the foregoing meanings;
or in that at least one compound of general formula (II),

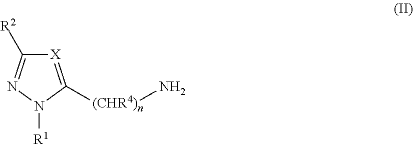

in which X, R¹, R², R⁴ and n have one of the foregoing meanings, is reacted to form a compound of general formula (V)

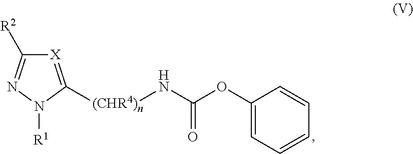

in which X, R¹, R², R⁴ and n have one of the foregoing meanings, in a reaction medium, in the presence of phenyl chloroformate, if appropriate in the presence of at least one base and/or at least one coupling reagent, and said compound is if appropriate purified and/or isolated, and a compound of general formula (V) is reacted with a compound of general formula (VI),

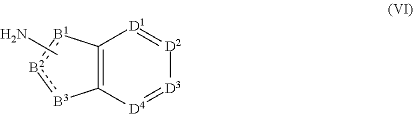

in which B¹, B², B³, D¹, D², D³ and D⁴ each have one of the foregoing meanings, in a reaction medium, if appropriate in the presence of at least one suitable coupling reagent, if appropriate in the presence of at least one base, to form a compound of general formula (I), in which A represents N and the other variables, substituents and indices have one of the foregoing meanings.

The corresponding thio-compounds, i.e. compounds of general formula (I) with Y representing S may be prepared in an analogous manner.

The reaction of compounds of the above-indicated general formulae (II) and (VI) with carboxylic acids of the above-indicated general formula (III) to form compounds of the above-indicated general formula (I) is carried out preferably in a reaction medium selected from the group consisting of diethyl ether, tetrahydrofuran, acetonitrile, methanol, ethanol, (1,2)-dichloroethane, dimethylformamide, dichloromethane and corresponding mixtures, if appropriate in the presence of at least one coupling reagent, preferably selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCl), diisopropylcarbodiimide, 1,1′-carbonyldiimidazole (CDI), N-[(dimethylamino)-1H-1,2,3-triazolo[4,5-b]pyridino-1-yl-methylene]-N-methylmethanaminium hexafluorophosphate N-oxide (HATU), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), N-hydroxybenzotriazole (HOBt) and 1-hydroxy-7-azabenzotriazole (HOAt), if appropriate in the presence of at least one organic base, preferably selected from the group consisting of triethylamine, pyridine, dimethylaminopyridine, N-methylmorpholine and diisopropylethylamine, preferably at temperatures of from −70° C. to 100° C.

Alternatively, the reaction of compounds of the above-indicated general formulae (II) and (VI) with carboxylic acid halides of the above-indicated general formula (IV), in which Hal represents a halogen as the leaving group, preferably a chlorine or bromine atom, to form compounds of the above-indicated general formula (I) is carried out in a reaction medium preferably selected from the group consisting of diethyl ether, tetrahydrofuran, acetonitrile, methanol, ethanol, dimethylformamide, dichloromethane and corresponding mixtures, if appropriate in the presence of an organic or inorganic base, preferably selected from the group consisting of triethylamine, dimethylaminopyridine, pyridine and diisopropylamine, at temperatures of from −70° C. to 100° C.

The compounds of the above-indicated formulae (II), (III), (IV), (V) and (VI) are each commercially available and/or can be prepared using conventional processes known to the person skilled in the art.

The reactions described hereinbefore can each be carried out under the conventional conditions with which the person skilled in the art is familiar, for example with regard to pressure or the order in which the components are added. If appropriate, the person skilled in the art can determine the optimum procedure under the respective conditions by carrying out simple preliminary tests. The intermediate and end products obtained using the reactions described hereinbefore can each be purified and/or isolated, if desired and/or required, using conventional methods known to the person skilled in the art. Suitable purifying processes are for example extraction processes and chromatographic processes such as column chromatography or preparative chromatography. All of the process steps described hereinbefore, as well as the respective purification and/or isolation of intermediate or end products, can be carried out partly or completely under an inert gas atmosphere, preferably under a nitrogen atmosphere.

The substituted compounds according to the invention of the aforementioned general formula (I) and also corresponding stereoisomers can be isolated both in the form of their free bases, their free acids and also in the form of corresponding salts, in particular physiologically compatible salts.

The free bases of the respective substituted compounds according to the invention of the aforementioned general formula (I) and also of corresponding stereoisomers can be converted into the corresponding salts, preferably physiologically compatible salts, for example by reaction with an inorganic or organic acid, preferably with hydrochloric acid, hydrobromic acid, sulphuric acid, methanesulphonic acid, p-toluenesulphonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulphonic acid, nicotinic acid, 2, 3 or 4-aminobenzoic acid, 2,4,6-trimethylbenzoic acid, α-lipoic acid, acetyl glycine, hippuric acid, phosphoric acid and/or aspartic acid. The free bases of the respective substituted compounds of the aforementioned general formula (I) and of corresponding stereoisomers can likewise be converted into the corresponding physiologically compatible salts using the free acid or a salt of a sugar additive, such as for example saccharin, cyclamate or acesulphame.

Accordingly, the free acids of the substituted compounds of the aforementioned general formula (I) and of corresponding stereoisomers can be converted into the corresponding physiologically compatible salts by reaction with a suitable base. Examples include the alkali metal salts, alkaline earth metals salts or ammonium salts [NHₓR₄₋ₓ]⁺, in which x=0, 1, 2, 3 or 4 and R represents a branched or unbranched C₁₋₄ alkyl residue.

The substituted compounds according to the invention of the aforementioned general formula (I) and of corresponding stereoisomers can if appropriate, like the corresponding acids, the corresponding bases or salts of these compounds, also be obtained in the form of their solvates, preferably in the form of their hydrates, using conventional methods known to the person skilled in the art.

If the substituted compounds according to the invention of the aforementioned general formula (I) are obtained, after preparation thereof, in the form of a mixture of their stereoisomers, preferably in the form of their racemates or other mixtures of their various enantiomers and/or diastereomers, they can be separated and if appropriate isolated using conventional processes known to the person skilled in the art. Examples include chromatographic separating processes, in particular liquid chromatography processes under normal pressure or under elevated pressure, preferably MPLC and HPLC processes, and also fractional crystallisation processes. These processes allow individual enantiomers, for example diastereomeric salts formed by means of chiral stationary phase HPLC or by means of crystallisation with chiral acids, for example (+)-tartaric acid, (−)-tartaric acid or (+)-10-camphorsulphonic acid, to be separated from one another.

The substituted compounds according to the invention of the aforementioned general formula (I) and corresponding stereoisomers and also the respective corresponding acids, bases, salts and solvates are toxicologically safe and are therefore suitable as pharmaceutical active ingredients in pharmaceutical compositions.

The present invention therefore further relates to a pharmaceutical composition containing at least one compound according to the invention of the above-indicated formula (I), in each case if appropriate in the form of one of its pure stereoisomers, in particular enantiomers or diastereomers, its racemates or in the form of a mixture of stereoisomers, in particular the enantiomers and/or diastereomers, in any desired mixing ratio, or respectively in the form of a corresponding salt, or respectively in the form of a corresponding solvate, and also if appropriate one or more pharmaceutically compatible auxiliaries.

These pharmaceutical compositions according to the invention are suitable in particular for vanilloid receptor 1-(VR1/TRPV1) regulation, preferably for vanilloid receptor 1-(VR1/TRPV1) inhibition and/or for vanilloid receptor 1-(VR1/TRPV1) stimulation, i.e. they exert an agonistic or antagonistic effect.

Likewise, the pharmaceutical compositions according to the invention are preferably suitable for the inhibition and/or treatment of disorders or diseases which are mediated, at least in some cases, by vanilloid receptors 1.

The pharmaceutical composition according to the invention is suitable for administration to adults and children, including toddlers and babies.

The pharmaceutical composition according to the invention may be found as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one substituted compound of the above-indicated formula (I), if appropriate in the form of one of its pure stereoisomers, in particular enantiomers or diastereomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally contains further physiologically compatible pharmaceutical auxiliaries which can for example be selected from the group consisting of excipients, fillers, solvents, diluents, surface-active substances, dyes, preservatives, blasting agents, slip additives, lubricants, aromas and binders.

The selection of the physiologically compatible auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragées, capsules, granules, pellets, drops, juices and syrups are preferably suitable for oral application; solutions, suspensions, easily reconstitutable dry preparations and also sprays are preferably suitable for parenteral, topical and inhalative application. The substituted compounds according to the invention used in the pharmaceutical composition according to the invention in a repository in dissolved form or in a plaster, agents promoting skin penetration being added if appropriate, are suitable percutaneous application preparations. Orally or percutaneously applicable preparation forms can release the respective substituted compound according to the invention also in a delayed manner.

The pharmaceutical compositions according to the invention are prepared with the aid of conventional means, devices, methods and process known in the art, such as are described for example in, Remington's Pharmaceutical Sciences”, A.R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula I may vary and is for example dependent on the patient's weight or age and also on the type of application, the indication and the severity of the disorder. Conventionally 0.001 to 100 mg/kg, preferably 0.05 to 75 mg/kg, particularly preferably 0.05 to 50 mg of at least one such compound according to the invention are applied per kg of the patient's body weight.

The pharmaceutical composition according to the invention is preferably suitable for the treatment and/or inhibition of one or more disorders selected from the group consisting of pain selected from the group consisting of acute pain, chronic pain, neuropathic pain and visceral pain; joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Particularly preferably, the pharmaceutical composition according to the invention is suitable for the treatment and/or inhibition of one or more disorders selected from the group consisting of pain, preferably of pain selected from the group consisting of acute pain, chronic pain, neuropathic pain and visceral pain; joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Most particularly preferably, the pharmaceutical composition according to the invention is suitable for the treatment and/or inhibition of pain, preferably of pain selected from the group consisting of acute pain, chronic pain, neuropathic pain and visceral pain, and/or urinary incontinence.

The present invention further relates to the use of at least one compound according to the invention and also if appropriate of one or more pharmaceutically compatible auxiliaries for the preparation of a pharmaceutical composition for vanilloid receptor 1-(VR1/TRPV1) regulation, preferably for vanilloid receptor 1-(VR1/TRPV1) inhibition and/or for vanilloid receptor 1-(VR1/TRPV1) stimulation.

Preference is given to the use of at least one substituted compound according to the invention and also if appropriate of one or more pharmaceutically compatible auxiliaries for the preparation of a pharmaceutical composition for the inhibition and/or treatment of disorders or diseases which are mediated, at least in some cases, by vanilloid receptors 1.

Particular preference is given to the use of at least one compound according to the invention and also if appropriate of one or more pharmaceutically compatible auxiliaries for the preparation of a pharmaceutical composition for the treatment and/or inhibition of one or more disorders selected from the group consisting of pain, preferably of pain selected from the group consisting of acute pain, chronic pain, neuropathic pain and visceral pain and joint pain.

Particular preference is given to the use of at least one compound according to the invention and also if appropriate of one or more pharmaceutically compatible auxiliaries for the preparation of a pharmaceutical composition for the treatment and/or inhibition of one or more disorders selected from the group consisting of hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particular preference is given to the use of at least one substituted compound according to the invention and also if appropriate of one or more pharmaceutically compatible auxiliaries for the preparation of a pharmaceutical composition for the treatment and/or inhibition of one or more disorders selected from the group consisting of pain, preferably of pain selected from the group consisting of acute pain, chronic pain, neuropathic pain and visceral pain; joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Particular preference is given to the use of at least one substituted compound according to the invention and also if appropriate of one or more pharmaceutically compatible auxiliaries for the preparation of a pharmaceutical composition for the treatment and/or inhibition of pain, preferably selected from the group consisting of acute pain, chronic pain, neuropathic pain and visceral pain, and/or urinary incontinence.

The present invention further relates to at least one substituted compound according to the invention and also if appropriate to one or more pharmaceutically compatible auxiliaries for vanilloid receptor 1-(VR1/TRPV1) regulation, preferably for vanilloid receptor 1-(VR1/TRPV1) inhibition and/or for vanilloid receptor 1-(VR1/TRPV1) stimulation.

Preference is given to at least one substituted compound according to the invention and also if appropriate to one or more pharmaceutically compatible auxiliaries for the inhibition and/or treatment of disorders or diseases which are mediated, at least in some cases, by vanilloid receptors 1.

Particular preference is given to at least one compound according to the invention and also if appropriate to one or more pharmaceutically compatible auxiliaries for the treatment and/or inhibition of one or more disorders selected from the group consisting of pain, preferably of pain selected from the group consisting of acute pain, chronic pain, neuropathic pain and visceral pain and joint pain.

Particular preference is given to at least one compound according to the invention and also if appropriate to one or more pharmaceutically compatible auxiliaries for the treatment and/or inhibition of one or more disorders selected from the group consisting of hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particular preference is given to at least one compound according to the invention and also if appropriate to one or more pharmaceutically compatible auxiliaries for the treatment and/or inhibition of one or more disorders selected from the group consisting of pain, preferably of pain selected from the group consisting of acute pain, chronic pain, neuropathic pain and visceral pain; joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Particular preference is given to at least one compound according to the invention and also if appropriate to one or more pharmaceutically compatible auxiliaries for the treatment and/or inhibition of pain, preferably selected from the group consisting of acute pain, chronic pain, neuropathic pain and visceral pain, and/or urinary incontinence.

The present invention further relates to at least one substituted compound according to the invention and also if appropriate to one or more pharmaceutically compatible auxiliaries for use in vanilloid receptor 1-(VR1/TRPV1) regulation, preferably for use in vanilloid receptor 1-(VR1/TRPV1) inhibition and/or for vanilloid receptor 1-(VR1/TRPV1) stimulation. Preference is given to at least one substituted compound according to the invention and also if appropriate to one or more pharmaceutically compatible auxiliaries for use in the inhibition and/or treatment of disorders or diseases which are mediated, at least in some cases, by vanilloid receptors 1.

Particular preference is given to at least one compound according to the invention and also if appropriate to one or more pharmaceutically compatible auxiliaries for use in the treatment and/or inhibition of one or more disorders selected from the group consisting of pain, preferably of pain selected from the group consisting of acute pain, chronic pain, neuropathic pain and visceral pain and joint pain.

Particular preference is given to at least one compound according to the invention and also if appropriate to one or more pharmaceutically compatible auxiliaries for use in the treatment and/or inhibition of one or more disorders selected from the group consisting of hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particular preference is given to at least one compound according to the invention and also if appropriate to one or more pharmaceutically compatible auxiliaries for use in the treatment and/or inhibition of one or more disorders selected from the group consisting of pain, preferably of pain selected from the group consisting of acute pain, chronic pain, neuropathic pain and visceral pain; joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Particular preference is given to at least one compound according to the invention and also if appropriate to one or more pharmaceutically compatible auxiliaries for use in the treatment and/or inhibition of pain, preferably selected from the group consisting of acute pain, chronic pain, neuropathic pain and visceral pain, and/or urinary incontinence.

### Pharmacological Methods

### I. Functional Testing Carried Out on the Vanilloid Receptor 1 (VRI/TRPV1 Receptor)

The agonistic or antagonistic effect of the substances to be tested on the rat-species vanilloid receptor 1 (VR1/TRPV1) can be determined using the following assay. In this assay, the influx of Ca²⁺ through the receptor channel is quantified with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA).

### Method:

Complete medium: 50 ml HAMS F12 nutrient mixture (Gibco Invitrogen GmbH, Karlsruhe, Germany) with
10% by volume of FCS (foetal calf serum, Gibco Invitrogen GmbH, Karlsruhe, Germany, heat-inactivated);

### 2 mM L-glutamine (Sigma, Munich, Germany);

1% by weight of AA solution (antibiotic/antimyotic solution, PAA, Pasching, Austria) and 25 ng/ml NGF medium (2.5 S, Gibco Invitrogen GmbH, Karlsruhe, Germany)
Cell culture plate: Poly-D-lysine-coated, black 96-well plates having a clear base (96-well black/clear plate, BD Biosciences, Heidelberg, Germany) are additionally coated with laminin (Gibco Invitrogen GmbH, Karlsruhe, Germany), the laminin being diluted with PBS (Ca—Mg-free PBS, Gibco Invitrogen GmbH, Karlsruhe, Germany) to a concentration of 100 μg/ml. Aliquots having a laminin concentration of 100 μg/ml are removed and stored at −20° C. The aliquots are diluted with PBS in a ratio of 1:10 to 10 μg/ml of laminin and respectively 50 μL of the solution are pipetted into a recess in the cell culture plate. The cell culture plates are incubated for at least two hours at 37° C., the excess solution is removed by suction and the recesses are each washed twice with PBS. The coated cell culture plates are stored with excess PBS which is not removed until just before the feeding of the cells.

### Preparation of the Cells:

The vertebral column is removed from decapitated rats and placed immediately into cold HBSS buffer (Hank's buffered saline solution, Gibco Invitrogen GmbH, Karlsruhe, Germany), i.e. buffer located in an ice bath, mixed with 1% by volume (percent by volume) of an AA solution (antibiotic/antimyotic solution, PAA, Pasching, Austria). The vertebral column is cut longitudinally and removed together with fasciae from the vertebral canal. Subsequently, the dorsal root ganglia (DRG) are removed and again stored in cold HBSS buffer mixed with 1% by volume of an AA solution. The DRG, from which all blood remnants and spinal nerves have been removed, are transferred in each case to 500 μL of cold type 2 collagenase (PAA, Pasching, Austria) and incubated for 35 minutes at 37° C. After the addition of 2.5% by volume of trypsin (PAA, Pasching, Austria), incubation is continued for 10 minutes at 37° C. After complete incubation, the enzyme solution is carefully pipetted off and 500 μL of complete medium are added to each of the remaining DRG. The DRG are respectively suspended several times, drawn through cannulae No. 1, No. 12 and No. 16 using a syringe and transferred to a 50 ml Falcon tube which is filled up to 15 ml with complete medium. The contents of each Falcon tube are respectively filtered through a 70 μm Falcon filter element and centrifuged for 10 minutes at 1,200 rpm and RT. The resulting pellet is respectively taken up in 250 μL of complete medium and the cell count is determined.

The number of cells in the suspension is set to 3×10⁵ per ml and 150 μL of this suspension are in each case introduced into a recess in the cell culture plates coated as described hereinbefore. In the incubator the plates are left for two to three days at 37° C., 5% by volume of CO₂ and 95% relative humidity. Subsequently, the cells are loaded with 2 μM of Fluo-4 and 0.01% by volume of Pluronic F127 (Molecular Probes Europe BV, Leiden, the Netherlands) in HBSS buffer (Hank's buffered saline solution, Gibco Invitrogen GmbH, Karlsruhe, Germany) for 30 min at 37° C., washed 3 times with HBSS buffer and after further incubation for 15 minutes at RT used for Ca²⁺ measurement in a FLIPR assay. The Ca²⁺-dependent fluorescence is in this case measured before and after the addition of substances (λex=488 nm, λem=540 nm). Quantification is carried out by measuring the highest fluorescence intensity (FC, fluorescence counts) over time.

### FLIPR Assay:

The FLIPR protocol consists of 2 substance additions. First the compounds to be tested (10 μM) are pipetted onto the cells and the Ca²⁺ influx is compared with the control (capsaicin 10 μM). This provides the result in % activation based on the Ca²⁺ signal after the addition of 10 μM of capsaicin (CP). After 5 minutes' incubation, 100 nM of capsaicin are applied and the Ca²⁺ influx is also determined.

Desensitising agonists and antagonists lead to suppression of the Ca²⁺ influx. The % inhibition is calculated compared to the maximum achievable inhibition with 10 μM of capsaicin.

Triple analyses (n=3) are carried out and repeated in at least 3 independent experiments (N=4).

Starting from the percentage displacement caused by different concentrations of the compounds to be tested of general formula I, IC₅₀ inhibitory concentrations which cause a 50-percent displacement of capsaicin were calculated. Kᵢ values for the test substances were obtained by conversion by means of the Cheng-Prusoff equation (Cheng, Prusoff; Biochem. Pharmacol. 22, 3099-3108, 1973).

### II. Functional Tests Carried Out on the Vanilloid Receptor (VR1)

The agonistic or antagonistic effect of the substances to be tested on the vanilloid receptor 1 (VR1) can also be determined using the following assay. In this assay, the influx of Ca²⁺ through the channel is quantified with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA).

### Method:

Chinese hamster ovary cells (CHO K1 cells, European Collection of Cell Cultures (ECACC) United Kingdom) are stably transfected with the VR1 gene. For functional testing, these cells are plated out on poly-D-lysine-coated black 96-well plates having a clear base (BD Biosciences, Heidelberg, Germany) at a density of 25,000 cells/well. The cells are incubated overnight at 37° C. and 5% CO₂ in a culture medium (Ham's F12 nutrient mixture, 10% by volume of FCS (foetal calf serum), 18 μg/ml of L-proline). The next day the cells are incubated with Fluo-4 (Fluo-4 2 μM, 0.01% by volume of Pluronic F127, Molecular Probes in HBSS (Hank's buffered saline solution), Gibco Invitrogen GmbH, Karlsruhe, Germany) for 30 minutes at 37° C. Subsequently, the plates are washed three times with HBSS buffer and after further incubation for 15 minutes at RT used for Ca²⁺ measurement in a FLIPR assay. The Ca²⁺-dependent fluorescence is measured before and after the addition of the substances to be tested (λex wavelength=488 nm, λem=540 nm). Quantification is carried out by measuring the highest fluorescence intensity (FC, fluorescence counts) over time.

### FLIPR Assay:

The FLIPR protocol consists of 2 substance additions. First the compounds to be tested (10 μM) are pipetted onto the cells and the Ca²⁺ influx is compared with the control (capsaicin 10 μM) (% activation based on the Ca²⁺ signal after the addition of 10 μM of capsaicin). After 5 minutes' incubation, 100 nM of capsaicin are applied and the Ca²⁺ influx is also determined.

Desensitising agonists and antagonists led to suppression of the Ca²⁺ influx. The % inhibition is calculated compared to the maximum achievable inhibition with 10 μM of capsaicin.

Starting from the percentage displacement caused by different concentrations of the compounds to be tested of general formula I, IC₅₀ inhibitory concentrations which cause a 50-percent displacement of capsaicin were calculated. K, values for the test substances were obtained by conversion by means of the Cheng-Prusoff equation (Cheng, Prusoff; Biochem. Pharmacol. 22, 3099-3108, 1973).

### III. Formalin Test Carried Out on Mice

In the formalin test, the testing to determine the antinociceptive effect of the compounds according to the invention is carried out on male mice (NMRI, 20 to 30 g body weight, Iffa, Credo, Belgium).

In the formalin test as described by D. Dubuisson et al., Pain 1977, 4, 161-174, a distinction is drawn between the first (early) phase (0 to 15 minutes after the injection of formalin) and the second (late) phase (15 to 60 minutes after the injection of formalin). The early phase, as an immediate reaction to the injection of formalin, is a model of acute pain, whereas the late phase is regarded as a model of persistent (chronic) pain (T. J. Coderre et al., Pain 1993, 52, 259-285). The corresponding descriptions in the literature are introduced herewith by way of reference and form part of the disclosure.

The compounds according to the invention are tested in the second phase of the formalin test to obtain information about the effects of substances on chronic/inflammatory pain.

The moment at which the compounds according to the invention are applied before the injection of formalin is selected as a function of the type of application of the compounds according to the invention. 10 mg of the test substances/kg of body weight are applied intravenously 5 minutes before the injection of formalin which is carried out by a single subcutaneous injection of formalin (20 μL, 1% aqueous solution) into the dorsal side of the right hind paw, thus inducing in free moving test animals a nociceptive reaction which manifests itself in marked licking and biting of the paw in question.

Subsequently, the nociceptive behaviour is continuously detected by observing the animals over a test period of three minutes in the second (late) phase of the formalin test (21 to 24 minutes after the injection of formalin). The pain behaviour is quantified by adding up the seconds over which the animals display licking and biting of the paw in question during the test period.

The comparison is carried out respectively with control animals which are given vehicles (0.9% aqueous sodium chloride solution) instead of the compounds according to the invention before the administration of formalin. Based on the quantification of the pain behaviour, the effect of the substance is determined in the formalin test as a percentage change relative to the corresponding control.

After the injection of substances having an antinociceptive effect in the formalin test, the described behaviour of the animals, i.e. licking and biting, is reduced or eliminated.

### IV. Testing of Analgesic Efficacy in the Writhing Test

The testing of analgesic efficacy in the compounds according to the invention of general formula I was carried out by phenylquinone-induced writhing in mice (modified in accordance with I. C. Hendershot and J. Forsaith (1959), J. Pharmacol. Exp. Ther. 125, 237-240). The corresponding description in the literature is introduced herewith by way of reference and forms part of the disclosure.

Male NMRI mice weighing from 25 to 30 g were used for this purpose. 10 minutes after intravenous administration of the compounds to be tested, groups of 10 animals per compound dose received 0.3 ml/mouse of a 0.02% aqueous solution of phenylquinone (phenylbenzoquinone, Sigma, Deisenhofen, Germany; solution prepared by adding 5% by weight of ethanol and storage in a water bath at 45° C.) applied intraperitoneally. The animals were placed individually into observation cages. A pushbutton counter was used to record the number of pain-induced stretching movements (what are known as writhing reactions=straightening of the torso with stretching of the rear extremities) for 5 to 20 minutes after the administration of phenylquinone. The control was provided by animals which had received only physiological saline solution. All the compounds were tested at the standard dosage of 10 mg/kg.

### V. Hypothermia Assay Carried Out on Mice

### Description of the Method:

The hypothermia assay is carried out on male NMRI mice (weight 25-35 grams, breeder IFFA CREDO, Brussels, Belgium). The animals were kept under standardised conditions: light/dark rhythm (from 6:00 to 18:00 light phase; from 18:00 to 6:00 dark phase), RT 19-22° C., relative humidity 35-70%, 15 room air changes per hour, air movement <0.2 m/sec. The animals received standard feed (ssniff R/M-Haltung, ssniff Spezialdi äten GmbH, Soest, Germany) and tap water. Water and feed were withdrawn during the experiment. All the animals were used only once during the experiment. The animals had an acclimatisation period of at least 5 days.

Acute application of capsaicin (VR-1 agonist) leads to a drop in the core temperature of the body in rats and mice due to stimulation of heat sensors. Only specifically effective VR-1 receptor antagonists can antagonise the capsaicin-induced hypothermia. By contrast, hypothermia induced by morphine is not antagonised by VR-1 antagonists. This model is therefore suitable for identifying substances with VR-1 antagonistic properties via their effect on body temperature.

Measurement of the core temperature was carried out using a digital thermometer (Thermalert TH-5, physitemp, Clifton N.J., USA). The sensing element is in this case inserted into the rectum of the animals.

To give an individual basic value for each animal, the body temperature is measured twice at an interval of approx. half an hour. One group of animals (n=6 to 10) then receives an intraperitoneal (i.p.) application of capsaicin 3 mg/kg and vehicle (control group). Another group of animals receives the substance to be tested (i.v. or p.o.) and additionally capsaicin (3 mg/kg) i.p. The test substance is applied i.v. 10 min, or p.o 15 minutes, prior to capsaicin. The body temperature is then measured 7.5/15 and 30 min following capsaicin (i.v.+i.p.) or 15/30/60/90/120 min (p.o.+i.p.) following capsaicin. In addition, one group of animals is treated with the test substance only and one group with vehicle only. The evaluation or representation of the measured values as the mean+/−SEM of the absolute values is carried out as a graphical representation. The antagonistic effect is calculated as the percentage reduction of the capsaicin-induced hypothermia.

### VI. Neuropathic Pain in Mice

Efficacy in neurotic pain was tested using the Bennett model (chronic constriction injury; Bennett and Xie, 1988, Pain 33: 87-107).

Three loose ligatures are tied around the right ischiadic nerve of Ketavet/Rompun-anaesthetised NMRI mice weighing 16-18 g. The animals develop hypersensitivity of the innervated paw caused by the damaged nerve, which hypersensitivity is quantified, following a recovery phase of one week, over a period of approximately three weeks by means of a cold metal plate (temperature 4° C.) (cold allodynia). The animals are observed on this plate over a period of 2 min and the withdrawal reactions of the damaged paw are counted. Based on the pre-value prior to the application of the substance, the substance's effect over a certain period of time is determined at various points in time (for example 15, 30, 45, or 60 min following application) and the resultant area under the curve (AUC) and/or the inhibition of cold allodynia at the individual measuring points is/are expressed as a percentage effect relative to the vehicle control (AUC) or to the starting value (individual measuring points). The group size is n=10, the significance of an antiallodynic effect (*=p<0.05) is determined with the aid of an analysis of variance with repeated measures and Bonferroni post hoc analysis.

The invention will be described hereinafter with the aid of a few examples. This description is intended merely by way of example and does not limit the general idea of the invention.

### EXAMPLES

The indication “equivalents” (“eq.”) means molar equivalents, “RT” means room temperature, “M” and “N” are indications of concentration in mol/l, “aq.” means aqueous, “sat.” means saturated, “sol.” means solution, “conc.” means concentrated.

Further abbreviations:

AcOH acetic acid
d days
bipy 2,2′-bipyridine/2,2′-bipyridyl
BOC/Boc tert.-butyloxycarbonyl
BOP 1-benzotriazolyloxy-tris-(dimethylamino)phosphonium hexafluorophosphate
brine saturated sodium chloride solution (NaCl sol.)
DCC N,N′-dicyclohexylcarbodiimide
DCM dichloromethane

### DIPEA N,N-diisopropylethylamine

### DMF N,N-dimethylformamide

DMAP 4-dimethylaminopyridine
EDC N-(3-dimethylaminopropyl)-N′-ethylcarbodiimide
EDCl N-ethyl-N′-(3-dimethylaminopropyl)carbodiimide hydrochloride
EE ethyl acetate
ether diethyl ether
EtOH ethanol
sat. saturated
h hour(s)
H₂O water

### HOBt N-hydroxybenzotriazole

LAH lithium aluminium hydride
LG leaving group
m/z mass-to-charge ratio
MeCN acetonitrile
MeOH methanol
min minutes
MS mass spectrometry
NA not available
NEt₃ triethylamine
RT/r.t./rt room temperature
R_{f} retention factor
SC silica gel column chromatography
THF tetrahydrofuran
TFA trifluoroacetic acid
TLC thin layer chromatography
vv volume ratio

The yields of the compounds prepared were not optimized. All temperatures are uncorrected. All starting materials which are not explicitly described were either commercially available (the details of suppliers such as for example Acros, Avocado, Aldrich, Bachem, Fluka, Lancaster, Maybridge, Merck, Sigma, TCl, Oakwood, etc. can be found in the Symyx® Available Chemicals Database of MDL, San Ramon, US, for example) or the synthesis thereof has already been described precisely in the specialist literature (experimental guidelines can be looked up in the Reaxys® Database of Elsevier, Amsterdam, NL, for example) or can be prepared using the conventional methods known to the person skilled in the art.

The stationary phase used for the column chromatography was silica gel 60 (0.0-0-0.063 mm) from E. Merck, Darmstadt. The thin-layer chromatographic tests were carried out using HPTLC precoated plates, silica gel 60 F 254, from E. Merck, Darmstadt. The mixing ratios of solvents, mobile solvents or for chromatographic tests are respectively specified in volume/volume.

All the intermediate products and example compounds were analytically characterized by means of ¹H-NMR spectroscopy. In addition, mass spectrometry tests (MS, m/z indication for [M+H]⁺) were carried out for all the example compounds and selected intermediate products.

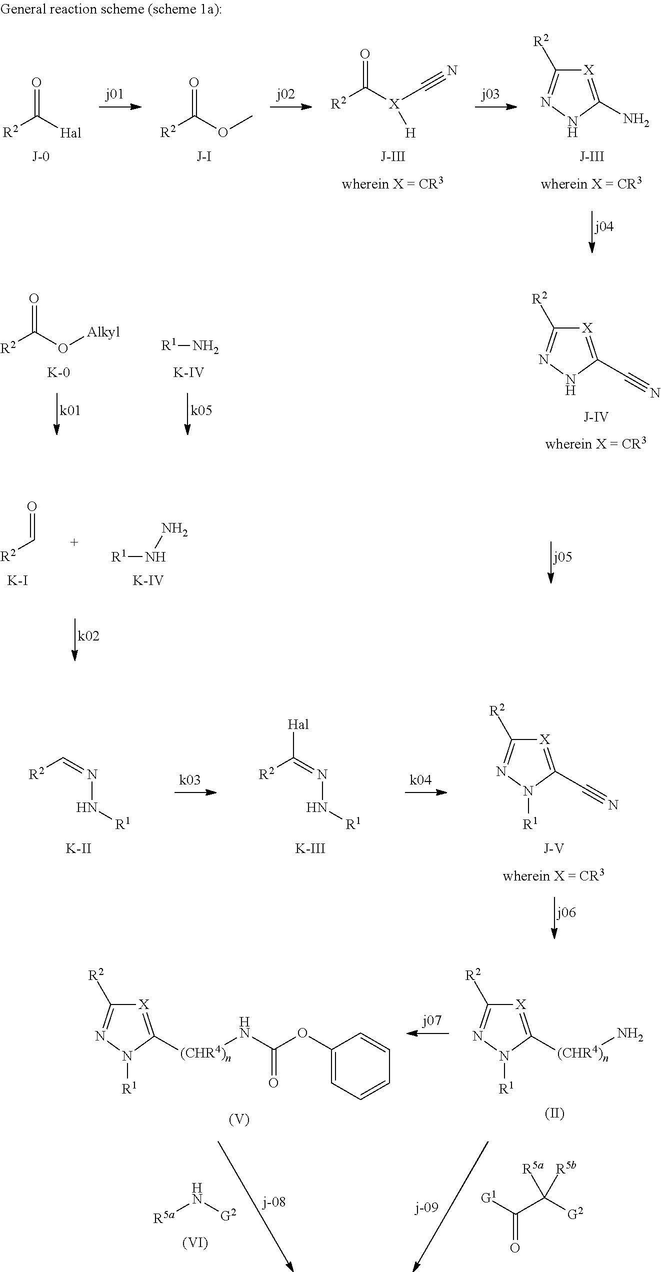

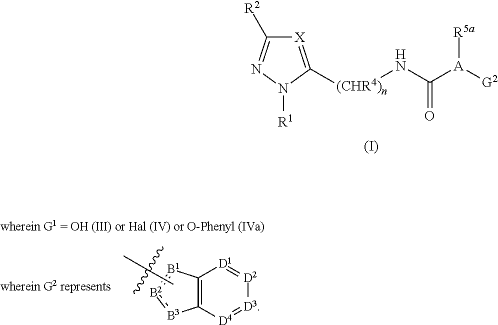

In step j01 an acid halide J-0, in which Hal preferably represents Cl or Br, can be esterified using methanol to form the compound J-I by means of methods with which the person skilled in the art is familiar.

In step j02 the methyl pivalate J-I can be converted into an oxoalkylnitrile J-II, wherein X═CR³, by means of methods known to the person skilled in the art, such as for example using an alkyl nitrile R³CH₂—CN, if appropriate in the presence of a base.

In step j03 the compound J-II can be converted into an amino-substituted pyrazolyl derivative J-III, wherein X═CR³, by means of methods known to the person skilled in the art, such as for example using hydrazine hydrate, with cyclisation.

In step j04 the amino compound J-III can first be converted into a diazonium salt by means of methods known to the person skilled in the art, such as for example using nitrite, and the diazonium salt can be converted into a cyano-substituted pyrazolyl derivative J-IV, wherein X═CR³, with elimination of nitrogen using a cyanide, if appropriate in the presence of a coupling reagent.

In step j05 the compound J-IV can be substituted in the N position by means of methods known to the person skilled in the art, for example using a halide R¹—Hal, if appropriate in the presence of a base and/or a coupling reagent, wherein Hal is preferably Cl, Br or I, or using a boronic acid B(OH)₂R¹ or a corresponding boronic acid ester, if appropriate in the presence of a coupling reagent and/or a base and the compound J-V, wherein X═CR³, can in this way be obtained. If R¹ is linked to general formula (I) via a heteroatom (if R¹ represents substructure (T1), for example, in which o represents 1 and G can represent inter alia O, S, S(═O)₂ or NR¹⁴), then the substitution can be carried out using methods known to the person skilled in the art, for example with the aid of hydroxylamine-O-sulphonic acid and subsequent conversion into secondary or tertiary amines, wherein G=NR¹⁴. In the case of G=O, the substitution can be carried out using methods known to the person skilled in the art, for example with the aid of peroxy reagents and subsequent conversion into ether. In the case of G=S(═O)₂, the substitution can be carried out by sulphonylation with sulphonyl chlorides, for example. In the case of G=S, the preparation can for example be carried out by reaction with disulphides or else with sulphenyl chlorides or sulphene amides, or else by transformation into the mercaptan by means of methods known to the person skilled in the art and subsequent conversion into the thioether.

Alternatively, a second synthesis pathway, in which in step k01 an ester K-0 is first reduced to form the aldehyde K-I by means of methods known to the person skilled in the art, for example using suitable hydrogenation reagents such as metal hydrides, is suitable for preparing the compound J-V, wherein X═CR³.

In step k02 the aldehyde K-I can then be reacted with a hydrazine K-V, which can be obtained in step k05, starting from the primary amine K-IV, by means of methods known to the person skilled in the art, to form the hydrazine K-II by means of methods known to the person skilled in the art with elimination of water.

In step k03 the hydrazine K-II can be halogenated, preferably chlorinated, by means of methods known to the person skilled in the art with the double bond intact, such as for example using a chlorination reagent such as NCS, and the compound K-III can in this way be obtained.

In step k04 the hydrazonoyl halide K-III can be converted into a cyano-substituted compound J-V, wherein X═CR³, by means of methods known to the person skilled in the art, such as for example using a halogen-substituted nitrile, with cyclisation.

In step j06 the compound J-V can be hydrogenated by means of methods known to the person skilled in the art, for example using a suitable catalyst such as palladium/activated carbon or using suitable hydrogenation reagents, and the compound (II) can in this way be obtained.

In step j07 the compound (II) can be converted into the compound (V) by means of methods known to the person skilled in the art, such as for example using phenyl chloroformate, if appropriate in the presence of a coupling reagent and/or a base. In addition to the methods disclosed in the present document for preparing unsymmetrical ureas using phenyl chloroformate, there are further processes with which the person skilled in the art is familiar, based on the use of activated carbonic acid derivatives or isocyanates, if appropriate.

In step j08 the amine (VI) can be converted into the urea compound (I) (wherein A=N). This can be achieved by reaction with (V) by means of methods with which the person skilled in the art is familiar, if appropriate in the presence of a base.

In step j09 the amine (II) can be converted into the amide (I) (wherein A=C—R^{5b}). This can for example be achieved by reaction with an acid halide, preferably a chloride of formula (IV) by means of methods with which the person skilled in the art is familiar, if appropriate in the presence of a base or by reaction with an acid of formula (III), if appropriate in the presence of a suitable coupling reagent, for example HATU or CDI, if appropriate with the addition of a base. Further, the amine (II) may be converted into the amide (I) (wherein A=C—R^{5b}) by reaction of a compound (IVa) by means of methods with which the person skilled in the art is familiar, if appropriate in the presence of a base.

For preparing compounds (II), wherein X═N, it is necessary to take a third synthesis route according to the general reaction scheme 1b. The compounds (II) which are then obtained,

wherein X═N, can subsequently be further reacted in accordance with the above-described steps j07-j09.

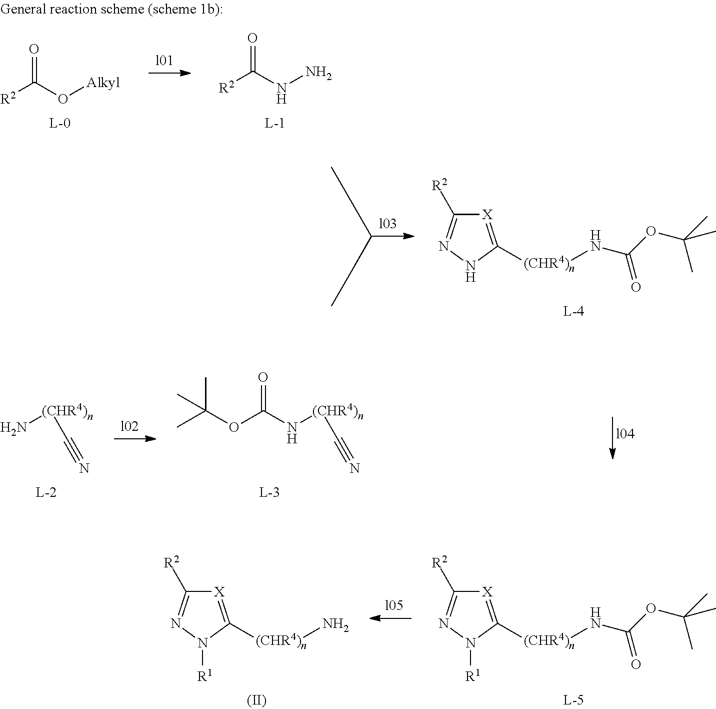

In step l01 a carboxylic acid alkyl ester L-0, preferably a methyl or ethyl ester, can be reacted with hydrazine hydrate to form the hydrazide L-1 by means of methods with which the person skilled in the art is familiar.

In step l02 the amino-substituted nitrile L-2 or the salts thereof can be reacted with boc anhydride to form the urethane L-3 by means of methods with which the person skilled in the art is familiar.

In step l03 L-1 and L-3 can be condensed in the presence of a base, preferably an alkali alcoholate, particularly preferably sodium methanolate, to form the triazole L-4, wherein X═N, by means of methods with which the person skilled in the art is familiar. In step l04 the compound L-4, wherein X═N, can be substituted in the N position by means of methods known to the person skilled in the art, in a manner similar to the step j05 according to general reaction scheme 1a by means of the methods described hereinbefore, and compound L-5, wherein X═N, can in this way be obtained.

In step l05 the ester group in L-4 can be eliminated in the presence of an acid, preferably trifluoroacetic acid or hydrochloric acid, by means of methods known to the person skilled in the art, and the amine (II) can in this way be obtained.

The compounds according to general formula (I), wherein A=N, may be further prepared by a reaction sequence according to general reaction scheme 1c.

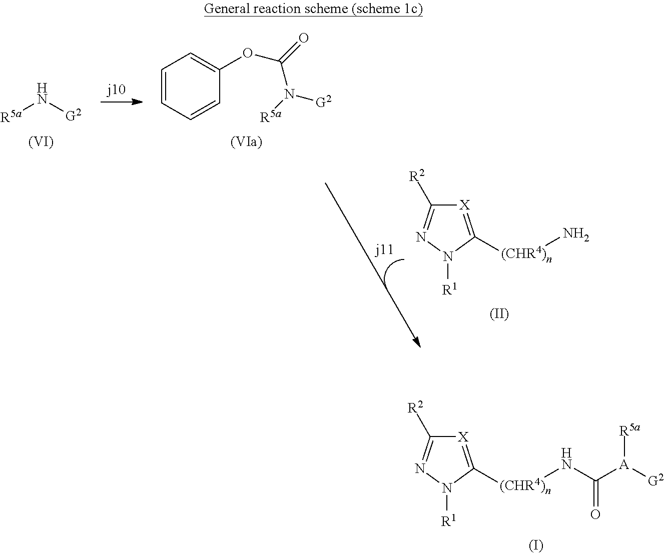

In step j10 the compound (VI) can be converted into the compound (VIa) by means of methods known to the person skilled in the art, such as for example using phenyl chloroformate, if appropriate in the presence of a coupling reagent and/or a base. In addition to the methods disclosed in the present document for preparing unsymmetrical ureas using phenyl chloroformate, there are further processes with which the person skilled in the art is familiar, based on the use of activated carbonic acid derivatives or isocyanates, if appropriate.

In step j11 the amine (II) can be converted into the urea compound (I) (wherein A=N). This can be achieved by reaction with (VIa) by means of methods with which the person skilled in the art is familiar, if appropriate in the presence of a base.

The methods with which the person skilled in the art is familiar for carrying out the reaction steps j01 to j09 and also k01 to k05 and l01 to l05 as well as j10 and j11 may be inferred from the standard works on organic chemistry such as, for example, J. March, Advanced Organic Chemistry, Wiley & Sons, 6th edition, 2007; F. A. Carey, R. J. Sundberg, Advanced Organic Chemistry, Parts A and B, Springer, 5th edition, 2007; team of authors, Compendium of Organic Synthetic Methods, Wiley & Sons. In addition, further methods and also literature references can be issued by the common databases such as, for example, the Reaxys® database of Elsevier, Amsterdam, NL or the SciFinder® database of the American Chemical Society, Washington, US.

### Synthesis of Intermediate Products

### 1. Synthesis of 3-tert-butyl-1-methyl-1H-pyrazol-5-yl-methanamine (steps j01-j06)

Step j01: Pivaloyl chloride (J-0) (1 eq., 60 g) was added dropwise to a solution of MeOH (120 ml) within 30 min at 0° C. and the mixture was stirred for 1 h at room temperature. After the addition of water (120 ml), the separated organic phase was washed with water (120 ml), dried over sodium sulphate and codistilled with dichloromethane (150 ml). The liquid product J-I was able to be obtained at 98.6% purity (57 g).

Step j02: NaH (50% in paraffin oil) (1.2 eq., 4.6 g) was dissolved in 1,4-dioxane (120 ml) and the mixture was stirred for a few minutes. Acetonitrile (1.2 eq., 4.2 g) was added dropwise within 15 min and the mixture was stirred for a further 30 min. The methyl pivalate (J-I) (1 eq., 10 g) was added dropwise within 15 min and the reaction mixture was refluxed for 3 h. After complete reaction, the reaction mixture was placed in iced water (200 g), acidified to pH 4.5 and extracted with dichloromethane (12×250 ml). The combined organic phases were dried over sodium sulphate, distilled and after recrystallisation from hexane (100 ml) 5 g of the product (J-II) (51% yield) was able to be obtained as a solid brown substance.

Step j03: At room temperature 4,4-dimethyl-3-oxopentanenitrile (J-II) (1 eq., 5 g) was taken up in EtOH (100 ml), mixed with hydrazine hydrate (2 eq., 4.42 g) and refluxed for 3 h. The residue obtained after removal of the EtOH by distillation was taken up in water (100 ml) and extracted with EE (300 ml). The combined organic phases were dried over sodium sulphate, the solvent was removed under vacuum and the product (J-III) (5 g, 89% yield) was obtained as a light red solid after recrystallisation from hexane (200 ml).

Step j04: 3-Tert-butyl-1H-pyrazol-5-amine (J-III) (1 eq., 40 g) was dissolved in dilute HCl (120 ml of HCl in 120 ml of water) and mixed dropwise with NaNO₂ (1.03 eq., 25 g in 100 ml) at 0-5° C. over a period of 30 min. After stirring for 30 minutes, the reaction mixture was neutralised with Na₂CO₃. A diazonium salt obtained by reaction of KCN (2.4 eq., 48 g), water (120 ml) and CuCN (1.12 eq., 31 g) was added dropwise to the reaction mixture within 30 min and the mixture was stirred for a further 30 min at 75° C. After complete reaction, the reaction mixture was extracted with EE (3×500 ml), the combined organic phases were dried over sodium sulphate and the solvent was removed under vacuum. The purification (SiO₂, 20% EE/hexane) of the residue by column chromatography produced a white solid (J-IV) (6.5 g, 15.1% yield).

### Step j05 (Method 1):

3-tert.-butyl-1H-pyrazol-5-carbonitrile (J-IV) (10 mmol) was added to a suspension of NaH (60%) (12.5 mmol) in DMF (20 ml) at room temperature while stirring. After stirring for 15 minutes, methyl iodide (37.5 mmol) was added dropwise to this reaction mixture at room temperature. After stirring for 30 min at 100° C., the reaction mixture was mixed with water (150 ml) and extracted with dichloromethane (3×75 ml). The combined organic extracts were washed with water (100 ml) and sat. NaCl solution (100 ml) and dried over magnesium sulphate. After removal of the solvent under vacuum, the residue was purified by column chromatography (SiO₂, various mixtures of EE and cyclohexane as the mobile solvent) and the product J-V was obtained.

### Step j06:

### Method 1:

J-V was dissolved together with palladium on carbon (10%, 500 mg) and concentrated HCl (3 ml) in MeOH (30 ml) and exposed to a hydrogen atmosphere for 6 hours at room temperature. The reaction mixture was filtered over celite and the filtrate was concentrated under vacuum. The residue was purified by means of flash chromatography (SiO₂, EE) and the product (II) was in this way obtained.

### Method 2:

J-V was dissolved in THF (10 ml) and BH₃.S(CH₃)₂ (2.0 M in THF, 3 ml, 3 equivalent) was added thereto. The reaction mixture was heated to reflux for 8 hours, aq. 2 N HCl (2 N) was added thereto and the reaction mixture was refluxed for a further 30 minutes. The reaction mixture was mixed with aq. NaOH solution (2N) and washed with EE. The combined organic phases were washed with sat. aq. NaCl solution and dried over magnesium sulphate. The solvent is removed under vacuum and the residue is purified by column chromatography (SiO₂, various mixtures of dichloromethane and methanol as the mobile solvent) and the product (II) (3-tert-butyl-1-methyl-1H-pyrazol-5-yl)methanamine) is in this way obtained.

### 2. The Following Further Intermediate Products can Synthesised in a Similar Manner Using the Process Described Hereinbefore Under 1

3-tert-butyl-1-hexyl-1H-pyrazol-5-yl-methanamine

### 3. Alternatively, Step j05 can Also be Carried Out as Follows (Method 2)

### Step j05 (Method 2):

A mixture of 3-tert-butyl-1H-pyrazol-5-carbonitrile (J-IV) (10 mmol), a boronic acid B(OH)₂R¹ or a corresponding boronic acid ester (20 mmol) and copper (II) acetate (15 mmol) is placed in dichloromethane (200 ml), mixed with pyridine (20 mmol) while stirring at room temperature and the mixture is stirred for 16 h. After removal of the solvent under vacuum, the residue obtained is purified by column chromatography (SiO₂, various mixtures of EE and cyclohexane as the mobile solvent) and the product J-V is in this way obtained.

The following further intermediate products were/can be prepared in this way (steps j01106):
(3-tert-butyl-1-(3-fluorophenyl)-1H-pyrazol-5-yl)methanamine
(3-tert-butyl-1-(3-chlorophenyl)-1H-pyrazol-5-yl)methanamine
(3-tert-butyl-1-(3-chloro-4-fluorophenyl)-1H-pyrazol-5-yl)methanamine
(3-tert-butyl-1-(4-methoxyphenyl)-1H-pyrazol-5-yl)methanamine

### 4. Synthesis of 1-(3-chlorophenyl)-3-(trifluoromethyl)-1H-pyrazol-5-yl-methanamine (Steps k01-k05 and j06)

Step k01: LAlH (lithium aluminium hydride) (0.25 eq., 0.7 g) was dissolved in dry diethyl ether (30 ml) under a protective gas atmosphere and stirred for 2 h at room temperature. The suspension obtained was taken up in diethyl ether (20 ml). Ethyl-2,2,2-trifluoroacetate (K-0) (1 eq., 10 g) was taken up in dry diethyl ether (20 ml) and added dropwise to the suspension at −78° C. over a period of 1 h. The mixture was then the stirred for a further 2 h at −78° C. EtOH (95%) (2.5 ml) was then added dropwise, the reaction mixture was heated to room temperature and placed on iced water (30 ml) with concentrated H₂SO₄ (7.5 ml). The organic phase was separated and concentrated under vacuum and the reaction product K-II was immediately introduced into the next reaction step k02.

Step k05: 3-chloroaniline (K-IV) (1 eq., 50 g) was dissolved at −5 to 0° C. in concentrated HCl (300 ml) and stirred for 10 min. A mixture of NaNO₂ (1.2 eq., 32.4 g), water (30 ml), SnCl₂.2H₂O (2.2 eq., 70.6 g) and concentrated HCl (100 ml) was added dropwise over a period of 3 h while maintaining the temperature. After stirring for a further 2 h at −5 to 0° C., the reaction mixture was set to pH 9 using NaOH solution and extracted with EE (250 ml). The combined organic phases were dried over magnesium sulphate and the solvent was removed under vacuum. The purification by column chromatography (SiO₂, 8% EE/hexane) produced 40 g (72% yield) of (3-chlorophenyl)hydrazine (K-IV) as a brown oil.

Step k02: The aldehyde (K-I) (2 eq., 300 ml) obtained from k01 and (3-chlorophenyl)hydrazine (K-IV) (1 eq., 20 g) were placed in EtOH (200 ml) and refluxed for 5 h. The solvent was removed under vacuum, the residue was purified by column chromatography (SiO₂, hexane) and the product (25 g, 72% yield) K-II was obtained as a brown oil.

Step k03: The hydrazine K-II (1 eq., 25 g) was dissolved in DMF (125 ml). N-chlorosuccinimide (1.3 eq., 19.5 g) was added portionwise at room temperature within 15 min and the mixture was stirred for 3 h. The DMF was removed by distillation and the residue was taken up in EE. The EE was removed under vacuum, the residue obtained was purified by column chromatography (SiO₂, hexane) and the product K-III (26.5 g, 92% yield) was obtained as a pink-coloured oil.

Step k04: At room temperature the hydrazonoyl chloride K-III (1 eq., 10 g) was taken up in toluene (150 ml) and mixed with 2-chloroacrylonitrile (2 eq., 6.1 ml) and TEA (2 eq., 10.7 ml). This reaction mixture was stirred for 20 h at 80° C. The mixture was then diluted with water (200 ml) and the phases were separated. The organic phase was dried over magnesium sulphate and the solvent was removed under vacuum. The residue was purified by means of column chromatography (SiO₂, 5% EE/hexane) and the product (5.5 g, 52% yield) was obtained as a white solid J-V.

### Step j06 (Method 3):

The carbonitrile J-V (1 eq., 1 g) was dissolved in methanolic ammonia solution (150 ml, 1:1) and hydrogenated in an H-cube (10 bar, 80° C., 1 ml/min, 0.25 mol/L). After removal of the solvent under vacuum, (1-(3-chlorophenyl)-3-(trifluoromethyl)-1H-pyrazol-5-yl)methanamine (II) was able to be obtained as a white solid (0.92 g, 91% yield).

### 5. The Following Further Intermediate Products were/can be Synthesized in a Similar Manner Using the Process Described Hereinbefore Under 4

(1-cyclohexyl-3-(trifluoromethyl)-1H-pyrazol-5-yl)methanamine
(1-(4-fluorophenyl)-3-(trifluoromethyl)-1H-pyrazol-5-yl)methanamine
(1-(3-chloro-4-fluorophenyl)-3-(trifluoromethyl)-1H-pyrazol-5-yl)methanamine
(1-(4-methoxyphenyl)-3-(trifluoromethyl)-1H-pyrazol-5-yl)methanamine
(1-(4-(trifluoromethoxy)phenyl)-3-(trifluoromethyl)-1H-pyrazol-5-yl)methanamine
(1-(3,4-dimethylphenyl)-3-(trifluoromethyl)-1H-pyrazol-5-yl)methanamine

### 6. Preparation of Selected Acids

### Synthesis of 2-(1-methyl-1H-indol-3-yl)propanoic acid (Examples 1, 2, 3, 15)

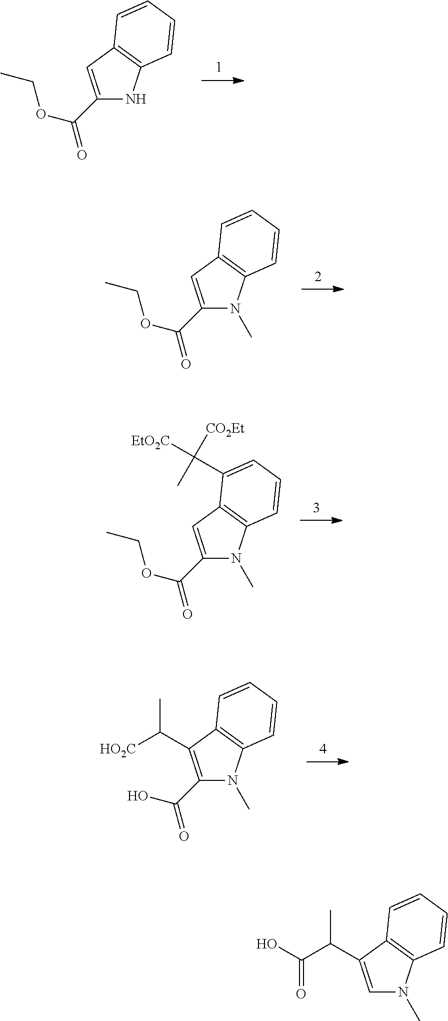

Step 1: To a stirred suspension of ethyl 1H-indole-2-carboxylate (10 g, 52.85 mmol), K₂CO₃ (21.9 g, 158.5 mmol) in acetonitrile (100 mL), dimethyl sulfate (7.54 mL, 79.27 mmol) was added at room temperature and the mixture was stirred at 90° C. for 6 h until complete consumption of the starting material. The reaction mixture was cooled to room temperature, filtered through celite pad to remove K₂CO₃, washed with ethyl acetate (2×25 mL). The filtrate was concentrated. The obtained residue was diluted with ethyl acetate (300 mL), washed with water (2×50 mL), brine solution (30 mL), dried over anhydrous Na₂SO₄, filtered and concentrated. The obtained crude compound was purified by column chromatography (100-200 mesh silica gel) using 6% EtOAc in petroleum ether as eluent to afford ethyl 1-methyl-1H-indole-2-carboxylate as a pale brown solid (10.6 g, 98% yield).

Step 2: To a stirred suspension of ethyl 1-methyl-1H-indole-2-carboxylate (10.7 g, 52.65 mmol), sodium acetate (21.58 g, 263.27 mmol) and diethyl methyl malonate (44.96 mL, 263.27 mmol) in AcOH (130 mL) at 0° C. and the reaction mixture was deoxygenated by purging with a stream of Argon for 30 min. Added Mn(OAc)₃.2H₂O (35.29 g, 131.62 mmol) and purging was continued for 10 min and stirred at 80° C. for 16 h. The reaction mixture was cooled to room temperature, quenched with brine solution (50 mL), extracted with ethyl acetate (2×200 mL). The combined ethyl acetate layer was washed with aq NaHCO₃ solution (2×50 mL), dried over anhydrous Na₂SO₄, filtered and concentrated under reduced pressure to afford diethyl 2-(2-(ethoxycarbonyl)-1-methyl-1H-indol-3-yl)-2-methylmalonate as pale brown solid (11 g, crude).

Step 3: To a stirred solution of KOH (3.2 g, 57.42 mmol) in EtOH (70 mL) and water (14 mL), added crude step-2 product (11 g, 29.33 mmol) at room temperature and the mixture was stirred at reflux for 4 h. The reaction mixture was acidified (pH-3) with 3 N HCl, diluted with water (75 mL), extracted with ethyl acetate (2×200 mL). The combined ethyl acetate layer was washed with brine solution (50 mL), dried over anhydrous Na₂SO₄, filtered and concentrated to afford 3-(1-carboxyethyl)-1-methyl-1H-indole-2-carboxylic acid as a brown solid (7 g, crude).

Step 4: A stirred solution of 3-(1-carboxyethyl)-1-methyl-1H-indole-2-carboxylic acid (7 g, crude, 28.34 mmol) in 6 N HCl (100 mL) was stirred at reflux for 1 h. The reaction mixture was diluted with water (50 mL), extracted with ethyl acetate (2×200 mL). The combined ethyl acetate layer was washed with brine solution (50 mL), dried over anhydrous Na₂SO₄, filtered and concentrated. The obtained crude compound was purified by column chromatography (100-200 mesh silica gel) using 5% MeOH in chloroform as eluent to afford 2-(1-methyl-1H-indol-3-yl)propanoic acid as a white solid (1.8 g, 17%).

### Synthesis of 2-(5-methoxy-1-methyl-1H-indol-3-yl)propanoic acid (Example 14)

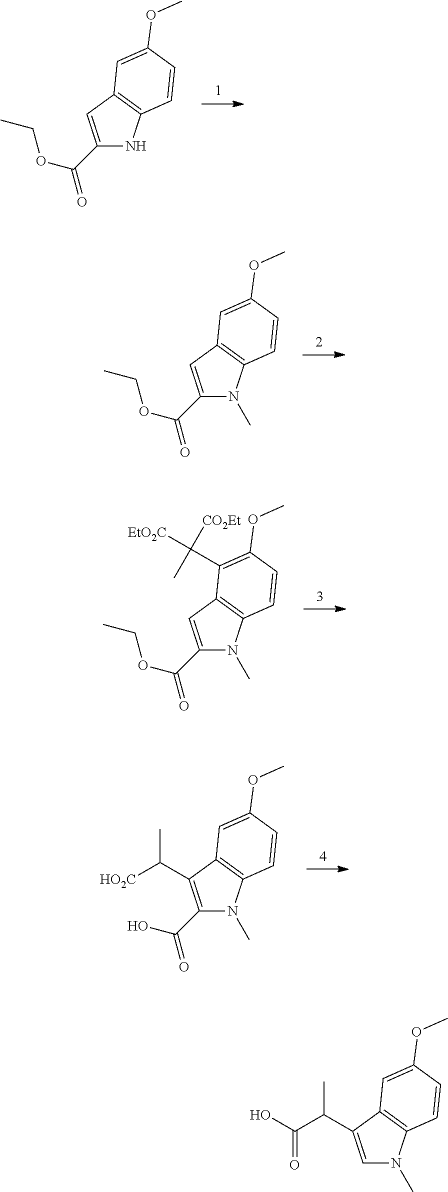

Step 1: Ethyl 5-methoxy-1H-indole-2-carboxylate (1 g, 4.56 mmol) was dissolved in acetonitrile (10 mL) followed by the addition of potassium carbonate (1.83 g, 13.68 mmol) and dimethyl sulfate (0.65 mL, 6.84 mmol). The resultant solution was heated to 90° C. for 6 h under nitrogen atmosphere. Potassium carbonate was filtered through sintered funnel and the filtrate was concentrated under reduced pressure. The residue was diluted with water (50 mL) and it was extracted with 20% ethyl acetate in hexane (3×20 mL). The combined organic layer was dried over anhydrous magnesium sulfate and concentrated under reduced pressure to afford crude material, which was purified by column chromatography (silica gel 100-200; eluent: 5% ethyl acetate in hexane) to afford pure ethyl 5-methoxy-1-methyl-1H-indole-2-carboxylate (970 mg, 91% yield).

Step 2: Ethyl 5-methoxy-1-methyl-1H-indole-2-carboxylate (970 mg, 4.16 mmol) was dissolved in acetic acid (22 mL). Sodium acetate (2.83 g, 20.8 mmol) and diethyl methyl malonate (3.6 mL, 20.8 mmol) were added to the reaction mixture under argon atmosphere. Manganese acetate dihydrate (2.8 g, 10.4 mmol) was added to the reaction mixture and the overall reaction mass was degassed and refilled with argon. It was heated to 80° C. for 16 h. Brine (30 mL) was added to the reaction mixture and it was extracted with 30% ethyl acetate in hexane (3×20 mL). The combined organic layer was washed with saturated Sodium bi carbonate solution (50 mL). The combined organic layer was dried over anhydrous magnesium sulfate and concentrated under reduced pressure to afford crude material, which was purified by column chromatography (silica gel 100-200; eluent: 20% ethyl acetate in hexane) to afford pure diethyl 2-(2-(ethoxycarbonyl)-5-methoxy-1-methyl-1H-indol-3-yl)-2-methylmalonate (1.43 g, 84% yield).

Step 3: Step-2 product (1.43 g, 3.53 mmol) was added in a solution of potassium hydroxide in ethanol-water (9.2: 1.8) mL. It was refluxed for 3 h. The reaction mixture was acidified with 3 N hydrochloric acid upto pH 3. It was diluted with water (20 mL). The aqueous part was extracted with ethyl acetate (3×20 mL). The combined organic layer was washed with brine (20 mL). It was dried over anhydrous magnesium sulfate and concentrated under reduced pressure to afford pure 3-(1-carboxyethyl)-5-methoxy-1-methyl-1H-indole-2-carboxylic acid (970 mg, 95% yield).

Step 4: Step-3 product (970 mg, 3.5 mmol) was taken in a 50 mL round-bottomed flask and 6 N hydrochloric acid (15 mL) was added to it. It was refluxed for 30 min. The reaction mixture was diluted with 40 mL water and extracted with 60% ethyl acetate in hexane (3×20 mL). The combined organic layer was washed with water (30 mL) and brine (30 mL). The combined organic layer was dried over anhydrous magnesium sulfate and concentrated under reduced pressure to afford crude compound which was purified by column chromatography (silica gel 100-200; eluent: 30% ethyl acetate in hexane) to afford pure 2-(5-methoxy-1-methyl-1H-indol-3-yl)propanoic acid (230 mg, 28% yield).

¹H NMR (DMSO-d₆, 400 MHz): δ 12.12 (s, 1H), 7.27 (d, 1H), 7.15 (s, 1H), 7.04 (s, 1H), 6.78 (d, 1H), 3.83 (q, 1H) 3.74 (s, 3H), 3.70 (s, 3H) 1.43 (d, 3H).

### Synthesis of 2-(5-hydroxy-1-methyl-1H-indol-3-yl)propanoic acid (example 4, 5, 6, 7, 10, 11, 12)

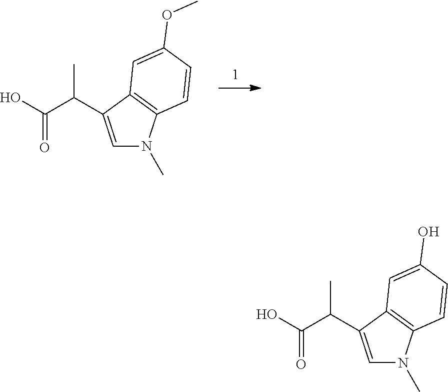

Step 1: 2-(5-Methoxy-1-methyl-1H-indol-3-yl)propanoic acid (1 g, 4.3 mmol) was dissolved in dichloromethane (25 mL) and it wags cooled to −10° C. Boron tribromide (5 mL, 5 mmol) was added slowly to the reaction mixture under nitrogen atmosphere. The reaction mixture was stirred for 3 h at room temperature. The reaction mixture was cooled and diluted with dichloromethane (50 mL) and quenched with ice. The aqueous part was extracted with dichloromethane (3×50 mL). The combined organic layer was dried over anhydrous magnesium sulfate and concentrated under reduced pressure to afford crude compound which was purified by column chromatography (silica gel 100-200; eluent: 30% ethyl acetate in hexane) to afford pure 2-(5-hydroxy-1-methyl-1H-indol-3-yl)propanoic acid (500 mg, 55% yield).

¹H NMR (DMSO-d₆, 400 MHz): δ 12.05 (s, 1H), 8.66 (s, 1H), 7.15 (d, 1H), 7.07 (s, 1H), 6.89 (s, 1H), 6.64 (d, 1H), 3.71 (q, 1H), 3.66 (s, 3H), 1.42 (d, 3H)

### Synthesis of 2-(5-hydroxy-1-methyl-1H-indol-3-yl)acetic acid (Examples 9, 18)

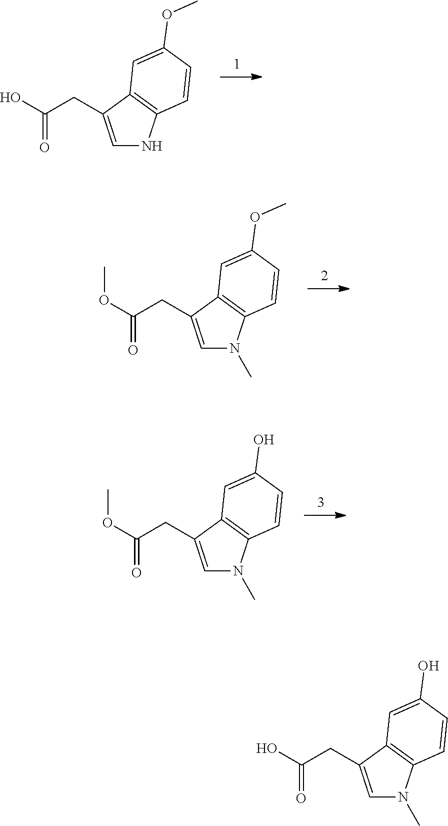

Step 1: To a well stirred and cooled suspension of 2-(5-methoxy-1H-indol-3-yl)acetic acid (1.95 g, 9.51 mmol) and KOH (3.18 g, 57.07 mmol) in acetone (60 mL) at 0° C., methyl iodide (3.56 mL, 57.07 mmol) was slowly added. The reaction mixture was stirred at room temperature for 20 h and concentrated under reduced pressure. The residue obtained was diluted with ethyl acetate (60 mL), washed with water (20 mL), 1N HCl (20 mL) and brine (20 mL), dried over anhydrous Na₂SO₄, and concentrated. The crude compound was purified by column chromatography (100-200 mesh silica gel) using 10% EtOAc in petroleum ether as eluent to get methyl 2-(5-methoxy-1-methyl-1H-indol-3-yl)acetate as white solid (2.10 g, 95% yield).

Step 2: To a stirred solution of 2-(5-methoxy-1-methyl-1H-indol-3-yl)acetate (1.6 g, 6.866 mmol) in dichloromethane (24 mL), 1 M solution of BBr₃ in dichloromethane (20.6 mL, 20.59 mmol) was added at 0° C. and stirred for 1 h at room temperature. The reaction mixture was quenched with alcohol (12 mL) at 0° C. and evaporated. The residue obtained was diluted with acetate (50 mL), washed with water (12 mL), saturated NaHCO₃ (12 mL) and brine (20 mL), dried over anhydrous Na₂SO₄ and evaporated. The crude compound was purified by column chromatography (100-200 mesh silica gel) using 30% EtOAc in petroleum ether as eluent to afford methyl 2-(5-hydroxy-1-methyl-1H-indol-3-yl)acetate as white solid (950 mg, 63% yield).

Step 3: To a stirred solution of LiOH.H₂O (546 mg, 13.01 mmol) in THF (20 mL) and water (6 mL), methyl 2-(5-hydroxy-1-methyl-1H-indol-3-yl)acetate (950 mg, 4.33 mmol) was added at 0° C. and stirred for 1 h at room temperature. The THF was evaporated, residue diluted with water (6 mL) and acidified (pH-3) with 1N HCl (6 mL), extracted with ethyl acetate (2×60 mL). The ethyl acetate layer was washed with brine solution (10 mL), dried over anhydrous Na₂SO₄ and concentrated. The crude thus obtained was purified by silica gel (100-200) using 5% MeOH in CHCl₃ as eluent to afford 2-(5-hydroxy-1-methyl-1H-indol-3-yl)acetic acid (0.74 g, 83% yield) as a white solid.

### Synthesis of 2-(5-methoxy-1-methyl-1H-indol-3-yl)acetic acid (Example 13)

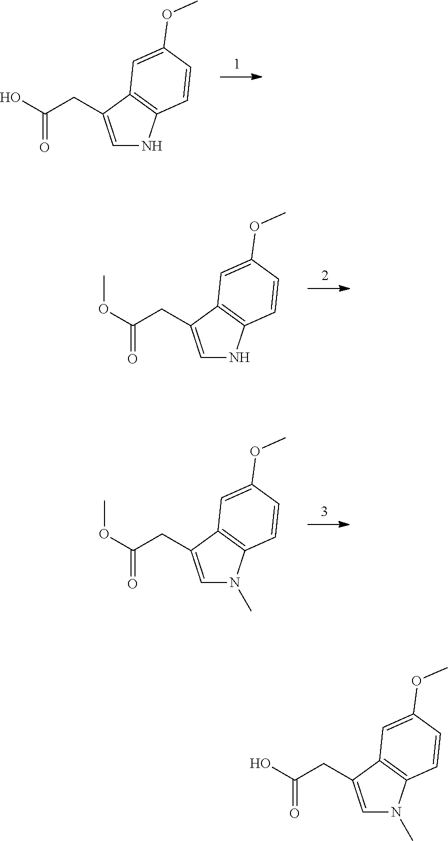

Step 1: To a stirred suspension of 2-(5-methoxy-1H-indol-3-yl)acetic acid (2 g, 9.75 mmol), K₂CO₃ (4.03 g, 29.24 mmol) in dimethylformamide (20 mL) at 0° C., methyl iodide (1.51 mL, 24.39 mmol) was added slowly and the mixture was stirred at room temperature for 2 h until complete consumption of starting material. The reaction mixture was diluted with water (20 mL), extracted with ethyl acetate (2×30 mL). The ethyl acetate layer was washed with water (2×10 mL), brine (10 mL), dried over anhydrous Na₂SO₄, filtered and concentrated. The obtained crude compound was purified by column chromatography (100-200 mesh silica gel) using 12% EtOAc in petroleum ether as eluent to afford methyl 2-(5-methoxy-1H-indol-3-yl)acetate (2.0 g, 94%) pale brown liquid. (TLC solvent system: 50% EtOAc-petroleum ether; R_{f}: 0.5; UV 254 nm), (VRR-A1740-102).

Step 2: To a stirred suspension of methyl 2-(5-methoxy-1H-indol-3-yl)acetate (2 g, 9.132 mmol), K₂CO₃ (5.04 g, 36.53 mmol) in acetonitrile (20 mL), dimethyl sulfate (2.6 mL, 27.39 mmol) was added at room temperature and the mixture was stirred at 85° C. for 16 h. The reaction mixture was cooled to room temperature, filtered through celite, washed with ethyl acetate (2×10 mL). The filtrate was concentrated. The obtained residue was diluted with ethyl acetate (100 mL), washed with water (2×20 mL), brine (10 mL), dried over anhydrous Na₂SO₄, filtered and concentrated. The obtained crude compound was purified by column chromatography (100-200 mesh silica gel) using 7% EtOAc in petroleum ether as eluent to afford methyl 2-(5-methoxy-1-methyl-1H-indol-3-yl)acetate (430 mg, 20% yield) as pale brown solid.

Step 3: To a stirred solution of LiOH.H₂O (310 mg, 7.38 mmol) in THF (15 mL) and water (4 mL), added crude methyl 2-(5-methoxy-1-methyl-1H-indol-3-yl)acetate (430 mg, 1.84 mmol) at room temperature and the mixture was stirred at this temperature for 2 h. THF was distilled off from the reaction mixture, the obtained residue was acidified (pH-3) with 3N HCl, diluted with water (10 mL), extracted with ethyl acetate (2×30 mL). The combined ethyl acetate layer was washed with brine solution (10 mL), dried over anhydrous Na₂SO₄, filtered and concentrated to afford 2-(5-methoxy-1-methyl-1H-indol-3-yl)acetic acid as a brown solid (400 g, 95% yield).

The respective acids used for the preparation of compounds according to example 16 and 17 are commercially available.

### Preparation of Selected Carbamate Phenyl Esters of General Formula (V)

### Synthesis of phenyl (3-tert-butyl-1-(3-chlorophenyl)-1H-pyrazol-5-yl)methylcarbamate

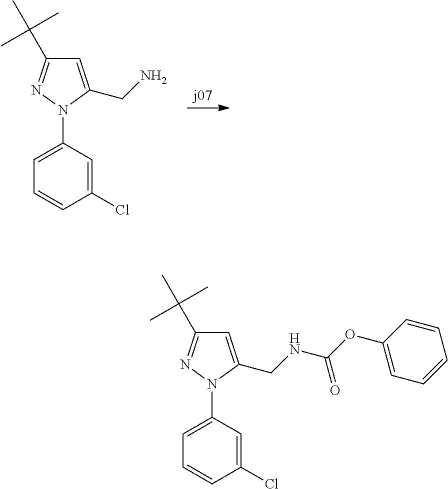

Schritt j07: To a solution of (3-tert-butyl-1-(3-chlorophenyl)-1H-pyrazol-5-yl)methanamine (5 g, 0.018 mol) in DMF (25 ml, 5 times), potassium carbonate (9.16 g, 0.066 mol, 3.5 eq) was added and cooled the contents to 0° C. Then phenyl chloroformate (3.28 g (2.65 ml), 0.02 mol, 1.1 eq) was added drop wise for 15 min and the overall reaction mixture was stirred for another 15 min at 0° C. Progress of the reaction was monitored by TLC (20% ethyl acetate/hexane, Rf˜0.3). On completion of the reaction, reaction contents were filtered, filtrate was diluted with cold water (100 ml) and the product extracted with ethyl acetate (3×25 ml). Combined organic layer was washed with brine solution (100 ml), dried over sodium sulfate and concentrated under reduced pressure. Crude obtained was purified by column chromatography (silica gel, 10% ethyl acetate/hexane) to yield the required product as a white solid (3.2 g, 45% yield).

### Synthesis of phenyl (1-(3-chlorophenyl)-3-(trifluoromethyl)-1H-pyrazol-5-yl)methylcarbamate (Employed for the Synthesis of Example Compounds no. 16 and 17)

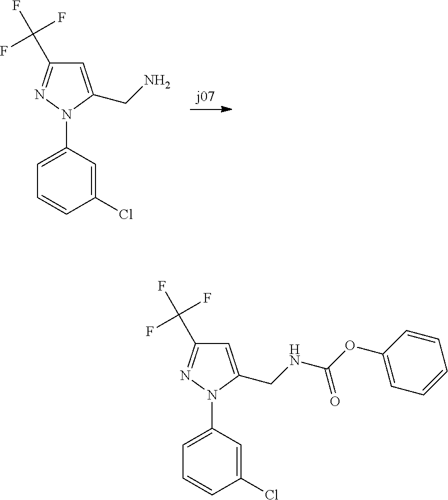

Step j07: To a solution of (3-tert-butyl-1-(3-chlorophenyl)-1H-pyrazol-5-yl)methanamine (2.5 g, 9.1 mmol, 1 eq) in dichloromethane (50 ml) was given phenyl chloroformate (1.28 mL, 10.2 mmol, 1.1 eq) and triethylamine 1.5 mL, 10.9 mmol, 1.2 Aq.). After 12 h stirring at room temperature the mixture was extracted with sodium carbonate solution (1×25 mL) and dichloromethane (2×25 mL). ethyl acetate (3×25 ml). Combined organic layer was dried over magnesium sulfate, concentrated under reduced pressure and the crude obtained was distilled under vacuum to yield the product as a white solid (2.9 g, 81% yield).

### Preparation of Additional Selected Pyrazol Derivatives According to General Formula (II) 9.1 Synthesis of (1-(3-chlorophenyl)-4-methyl-3-(trifluoromethyl)-1H-pyrazol-5-yl)methanamine (Employed for the Synthesis of Example Compound no. 7)

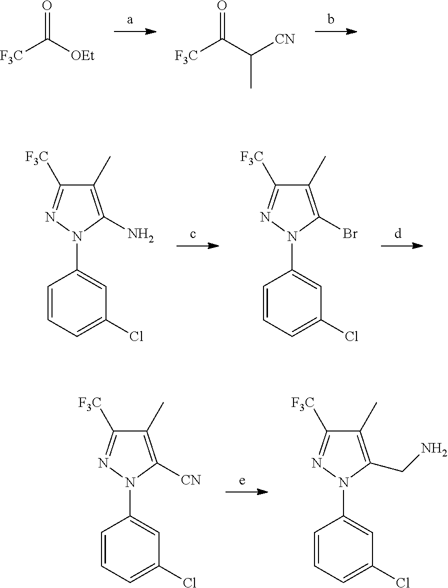

Step a: To a solution of diispropylamine (40.8 g (57 ml), 0.404 mol, 2.3 eq) in THF (400 ml), n-BuLi (1.6 molar) (24.7 g (258.3 ml, 0.38 mol, 2.2 eq) was added drop wise for 2 hrs at −20° C. and stirred the contents for 30-45 min at 0° C. Cooled the contents to −75° C., a solution of ethyl 2,2,2-trifluoroacetate (25 g, 0.17 mol) in THF (200 ml) was added drop wise for 2 hrs. The reaction mixture was stirred initially for 1 hr at −75° C. and later for another 1 hr at rt. Progress of the reaction was monitored by TLC (50% ethyl acetate/hexane, R_{f}˜0.5). On completion of the reaction, quenched the reaction with ice water (700 ml) and the solvents were distilled off completely. Residue washed with dichloromethane (3×300 ml), acidified the contents with 30% HCl solution and the product extracted with ether (3×400 ml). Combined organic layer was dried over sodium sulfate, concentrated under reduced pressure and the crude obtained was distilled under vacuum to yield the product at 35° C./0.1 mm as a colorless liquid (17 g, 64% yield).

Step b: A step-a product (10 g, 0.066 mol) was taken in ethanolic HCl (300 ml, 30 times) and 3-chlorophenyl hydrazine (9.43 g, 0.066 mol, 1 eq) was added. The reaction mixture was heated to reflux for 2 hrs. Progress of the reaction was monitored by TLC (20% ethyl acetate/hexane, R_{f}˜0.3). On completion of the reaction, reaction contents were concentrated and the residue taken in water (200 ml). Basified the contents to a pH˜12 with 1N NaOH solution and filtered the contents. Solid obtained was taken in ethyl acetate (200 ml), dried the contents over sodium sulfate and concentrated under reduced pressure to yield the required product as a red colored solid (12 g, 65% yield).

Step c: Cupric bromide (11.33 g, 0.0511 mol, 1.2 eq) was taken in acetonitrile (176 ml) and heated to 150° C. Then n-butyl nitrite (6.59 g (7.47 ml), 0.063 mol, 1.5 eq) was added followed by a solution of step-b product (11.75 g, 0.042 mol) in acetonitrile (176 ml) was added drop wise for 30 min at 150° C. and stirred for 15 min. Progress of the reaction was monitored by TLC (5% ethyl acetate/hexane, R_{f}˜0.7). On completion of the reaction, acetonitrile was distilled off, residue was taken in ice cold water (300 ml) and the product extracted with ethyl acetate (5×100 ml). Combined extract was dried over sodium sulfate, concentrated under reduced pressure and the crude obtained was subjected to column chromatography (silica gel, pure hexane). Pure product was not isolated and a mixture was obtained as a red colored liquid (16 g, crude) and the same product used for the next step.

Step d: To a solution of step-c product (13 g, 0.038 mol) in NMP (130 ml, 10 times), copper cyanide (6.8 g, 0.076 mol, 2 eq), sodium iodide (100 mg, catalytic) were added. The reaction mixture was placed in a pre-heated oil bath at 180° C. and allowed to stir for 8 hr. Progress of the reaction was monitored by TLC (5% ethyl acetate/hexane, R_{f}˜0.4). On completion of the reaction, diluted the reaction contents with water (200 ml) and the product extracted with ethyl acetate (5×100 ml). Combined extract was washed with cold water (5×50 ml), dried over sodium sulfate and concentrated under reduced pressure. The crude obtained was purified by column chromatography (silica gel, 2% ethyl acetate/hexane) to yield the required product as a pale yellow colored solid (8 g).

Step e: To a solution of step-d product (5 g, 0.017 mol) in dry THF (30 ml, 6 times), Boran-THF in THF (70 ml) was added drop wise for 30 min at 0-5° C. Reaction mixture was slowly heated to 50° C. and allowed to stir for 12 hrs. Progress of the reaction was monitored by TLC (75% ethyl acetate/hexane, R_{f}˜0.2). On completion of the reaction, acidified the contents to 0-5° C. with conc.HCl at 0° C. and stirred the contents for 2 hrs at rt. Then basified the contents to a pH-12 with 10% NaOH solution and the product extracted with ethyl acetate (5×50 ml). Combined extract was dried over sodium sulfate and concentrated under reduced pressure. Solid obtained was washed with 10% ether/hexane and dried to yield the required product as a white colored solid (3 g, 59% yield, mp 82-86° C.).

### Synthesis of (1-(3-chlorophenyl)-3-cyclopropyl-1H-pyrazol-5-yl)methanamine (Employed for the Synthesis of Example Compound no. 12)

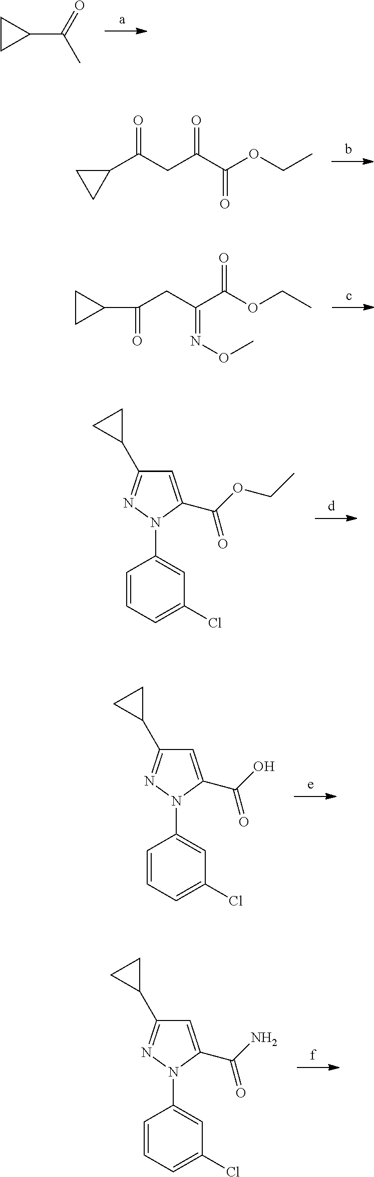

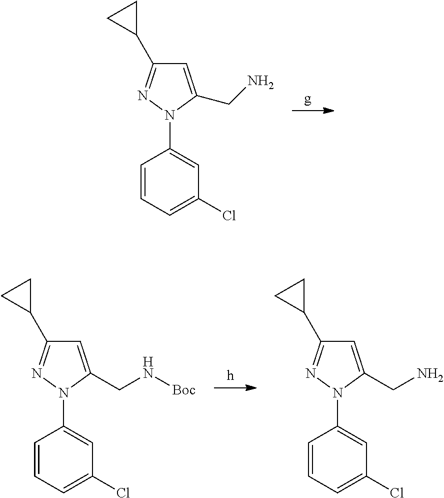

Step a: Sodium metal was dissolved into a solution of EtOH (150 ml) at RT under nitrogen atmosphere to form NaOEt (16.19 gm). This mixture was cooled to 0° C. Diethyl oxalate (34.76 gm) and isopropyl methyl ketone (20 gm) was added drop wise for about 15 min and warmed to RT. Now EtOH (100 ml) was added and stirred at RT for about 1 hour. Heat this reaction mixture to 80° C. for about 45 minutes and cooled to RT and concentrated under reduced pressure. To this resulting solid, add EtOAC. Wash with EtOH and filtered on cloth to get fine smooth powder. This solid is dissolved in water and acidified with dilute Sulphuric acid (pH-2). This compound is extracted with diethyl ether and dried over sodium sulphate and was concentrated under reduced pressure to obtain the brown colored liquid compound (40 g, 93% yield).

Step b: To a solution of step-a product (40 g) taken in ethanol (200 ml, 5 times), molecular sieves (40 g) was added at RT and stirred under nitrogen atmosphere for few minutes. keto ester was added at RT under nitrogen atmosphere and stirred the reaction for 12 hrs at RT. Progress of the reaction was monitored by TLC (10% ethyl acetate/hexane,). On completion of the reaction, filtered the reaction contents with EtOH or MeOH and the filtrate was distilled under reduced pressure. Residue obtained was dissolved in water (100 ml) and extracted with ethyl acetate (300 ml). Combined extract was dried over sodium sulfate and distilled under reduced pressure to obtain the crude product as brownish liquid (40 g). The crude obtained was used for the next step directly.

Step c: To a stirred solution of step-b compound (40 g, 0.18 mol) in a 1:1 mixture of acetic acid and ethanol (400 ml, 10 times) was dissolved at RT. To this reaction mixture 3-chlorophenylhydrazine (32.07 g, 1.2 eq) was added and stirred for about 10 minutes. The overall reaction was heated and reflux for 24 hrs. Progress of the reaction was monitored by TLC (10% ethyl acetate/hexane, 30% ethyl acetate/hexane). On completion of the reaction, Acetic acid and ethanol was distilled off under reduced pressure. Obtained crude was added to water (200 ml) and the extract was added to EtOAc (350 ml) to get separate layers. The organic layer obtained was dried over sodium sulfate and concentrated under reduced pressure. The crude compound brown colored liquid was obtained (33 g).

Step d: To a stirred solution of step-c product (16 g, 0.055 mol) in methanol (160 ml, 10 times), a solution of NaOH (6.6 g, 0.165 mol, 3 eq) in water (32 ml, 2 times) was added. The overall reaction was stirred for 5 minutes at RT. Progress of the reaction was monitored by TLC (50% ethyl acetate/hexane). On completion of the reaction, methanol and water were distilled off under reduced pressure. Add water (100 ml) to this compound and neutralize it with dilute with HCl (pH˜4). Then the contents were extracted with dichloromethane (250 ml) and the layers were separated. The Combined dichloromethane was dried over sodium sulfate and distilled under reduced pressure. The crude was obtained as white colored solid (13.5 g, 93.36% yield).

Step e: To a stirred solution of step-d product (11.5 g), dichloromethane (115 ml, 10 times) was added. The overall reaction was cooled to 0-5° C. At 0-5° C., SOCl₂ (3800 mL, 1.2 eq) was added by dropping funnel for about 10 min. The overall reaction was stirred for 3 h at room temperature. Progress of the reaction was monitored by TLC (50% ethyl acetate/hexane). On completion of the reaction, dichloromethane and SOCl₂ were distilled off under reduced pressure. Again add dichloromethane to this compound and stirred at RT. Then this solution was added drop wise to the solution of NH₃ in dichloromethane and maintained at 0-5° C. for 15 min and leave the reaction to get room temperature. This reaction mixture was stirred for overnight and the progress of the reaction was monitored by TLC (50% ethyl acetate/hexane). On completion of the reaction, dichloromethane was distilled off under reduced pressure. Again add dichloromethane (200 ml) and washed with cooled water (200 ml). and the layers were separated. The combined dichloromethane layer was dried over sodium sulfate and distilled under reduced pressure. The crude compound was obtained as white colored solid (11.0 g, 96% yield).

Step f: To a stirred solution of step-e product (11 g), amide and THF (110 ml, 10 times) was added. This reaction mixture was dried at. RT and cooled to 0-5° C. BH₃DMS (189.14 ml) and THF (14.37 gm, 4.5 eq) were added carefully drop wise by dropping funnel for about 1 hr. The overall reaction mass was maintained and reflux for about 24 hrs. The progress of the reaction was monitored by TLC (50% ethyl acetate/hexane). On completion of the reaction, mixture was cooled to 0° C. and quenched with diluted HCl (5M) and keep the reaction mixture undisturbed at RT for about 12 hrs. This compound was basidified with NaOH solution to Ph˜10. Then the contents were extracted with IPA/CHCl₃ and the layers were separated. The organic layer was dried over sodium sulfate and distilled under reduced pressure. The crude obtained is a brownish colored solid (11.4 g).

Step g: To a stirred solution of step-f product (11.4 g), dichloromethane (114 ml, 10 times), was added at RT and stirred for about 10 min. This reaction mixture was cooled to 0-5° C. in ice cold water. BOC-anhydride was added drop wise to the reaction mixture for about 15 min. Progress of the reaction was monitored by TLC (10% ethyl acetate/hexane/50% ethyl acetate/hexane). On completion of the reaction, added water (50 ml) and stirred the layer were separated. The organic layer was washed with water and the layers were separated. The organic layer was dried over sodium sulfate and distilled of under reduced pressure. The compound was obtained white colored solid (6.5 g, 40.6% yield).

Step h: To a stirred solution of Boc-compound (9.0 g), dichloromethane (100 ml) was added at RT and stirred for about 10 min. This reaction mixture was cooled to 0-5° C. and pass the HCl gas for about 20-30 min. Progress of the reaction was monitored by TLC (10% ethyl acetate/hexane/50% ethyl acetate/hexane). On completion of the reaction, distill off dichloromethane. Add water (100 ml) then extract the compound with 20% IPA/CHCl₃ and the layer were separated. The organic layer was distilled off under reduced pressure and dried under high vacuum. The crude was obtained by washing with heptane and drying under high vacuum. The compound was obtained light yellow colored viscous liquid (0.5 g, 78% yield).

### Synthesis of (3-tert-butyl-1-(pyridin-2-34)-1H-pyrazol-5-yl)methanamine (Employed for the Synthesis of Example Compound no. 6)

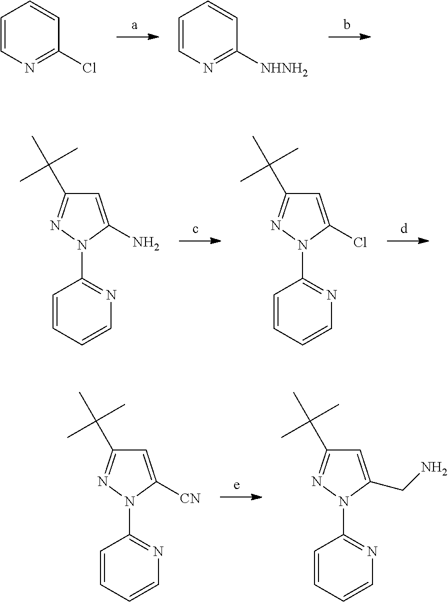

Step a: To a solution of 2-chloropyridine (20 g, 0.17 mol) in ethanol (100 ml, 5 times), hydrazine hydrate (132 ml, 6.6 times) was added and the reaction mixture was heated to reflux for 15 hrs. Progress of the reaction was monitored by TLC (40% ethyl acetate/hexane, Rf˜0.1). As the reaction not completed, continued to reflux for another 15 hrs and monitored by TLC. On completion of the reaction, ethanolic hydrazine hydrochloride was distilled off completely at 100° C., residue was taken in dichloromethane (500 ml) and washed the contents with saturated sodium carbonate solution (100 ml). Combined organic layer was dried over sodium sulfate and concentrated under reduced pressure to obtain the crude product as a low melting solid (11 g, crude). The crude obtained was directly used for the next step.

Step b: To a stirred solution of step-a product (11 g, crude) in ethanol (110 ml, 10 times), 4,4-dimethyl-3-oxopentanenitrile (11.3 g, 0.09 mol, 0.9 eq) was added portion wise followed by catalytic amount of HCl. The reaction mixture was heated to 100° C. and refluxed for 6 hrs. Progress of the reaction was monitored by TLC (20% ethyl acetate/hexane, Rf˜0.7). On completion of the reaction, ethanol was distilled off, residue was taken in water (200 ml) and the product extracted with ethyl acetate (2×100 ml). Combined extract was dried over sodium sulfate, concentrated under reduced pressure and the crude obtained was purified by column chromatography (silica gel, 10% ethyl acetate/hexane) to yield the required product as an off white solid (18 g).

### Synthesis of (1-(cyclopropylmethyl)-3-(trifluoromethyl)-1H-pyrazol-5-yl)methanamine (Employed for the Synthesis of Example Compound no. 15)

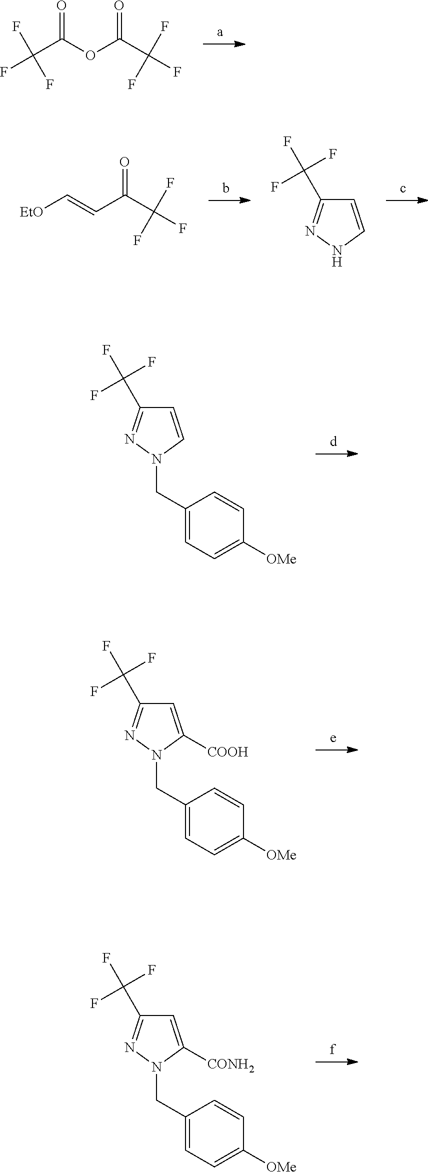

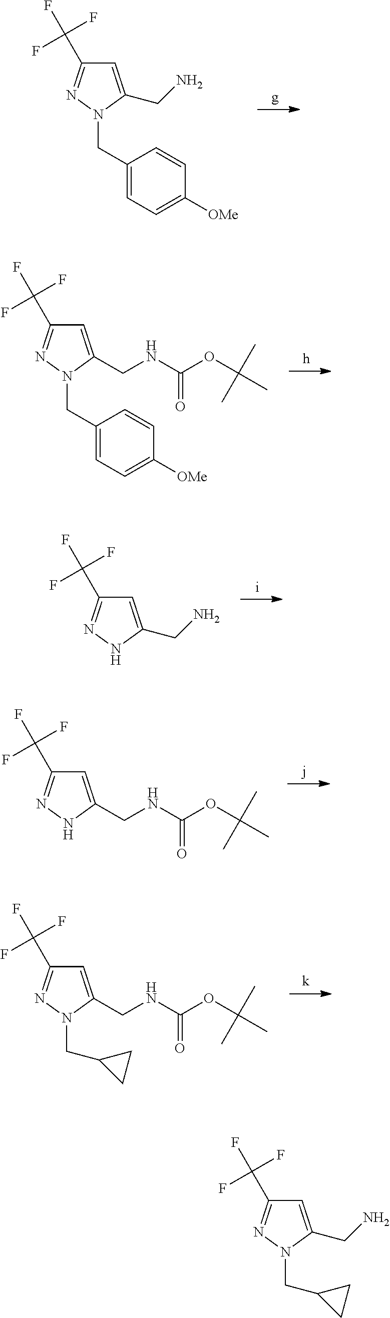

Step a: DMAP (4.25 g, 34 mmol, 0.01 eq) in dichloromethane (3000 mL) were charged into the flask and cooled to −10° C. Trifluoroacetic anhydride (765 g, 3200 mmol, 1.05 eq) was added followed by ethyl vinyl ether (250 g, 3040 mmol) was added drop wise for 45 min at −10° C. Then the overall reaction mixture was stirred for 8 h at 0° C. and for overnight at room temperature. On completion of the reaction, reaction contents were treated with saturated NaHCO₃ solution (600 mL) and organic layer was separated. Aqueous layer was extracted with dichloromethane (2×500 mL). Combined organic layer was washed with water (2×1000 mL), dried over sodium sulfate and concentrated under reduced pressure to give the crude product as a brown colored liquid (450 g, crude).

Step b: Hydrazine dihydrochloride (225 g, 2140 mmol, 1.6 eq) in ethanol (1400 mL) was stirred well. Triethylamine (135.4 g (185.4 mL), 1340 mmol, 1 eq) was added drop wise for 45 min at ambient temperature. Then (E)-4-ethoxy-1,1,1-trifluorobut-3-en-2-one (225 g, crude) was added drop wise at room temperature and the overall reaction mixture was refluxed for over night. On completion of the reaction, ethanol was distilled off completely, residue was taken in ice water (500 mL) and the product extracted with ethyl acetate (2×400 ml). Combined extract was washed with ice water (300 ml), dried over sodium sulfate and concentrated under reduced pressure to yield the required product as an off white solid (175 g, crude).

Step c: NaH (33.08 g (19.85, 60%), 1.5 eq) was washed with hexane, dry DMF (500 ml) was added drop wise under N₂ atmosphere and stirred well. A solution of 3-(trifluoromethyl)-1H-pyrazole (75 g, 550 mmol) in DMF (125 ml) was added drop wise under N₂ atmosphere. Then a solution of 4-methoxyl benzyl chloride (86.3 g, 550 mmol, 1 eq) in DMF (125 ml) was added drop wise and the overall reaction mixture was allowed to stir for 12 h at room temperature. On completion of the reaction, reaction contents were poured into ice water (500 ml) and the product was extracted with ethyl acetate (2×400 ml). The ethyl acetate layer was washed with 2N HCl (2×200 ml). Then the contents were dried over sodium sulfate and concentrated under reduced pressure. Obtained crude was purified by silica gel column chromatography with 10% ethyl acetate/Hexane to yield the required product as a brown colored liquid (98 g, 70% yield).

Step d: Diisopropyl amine (28.4 g (39.4 ml), 1.2 eq) was taken in THF (500 ml), stirred well and cooled the contents to 0° C. n-BuLi (234.4 ml, 1.5 eq) was added drop wise at 0° C. and stirred the contents for 1 h at 0° C. Then cooled the contents to −78° C., a solution of 1-(4-methoxybenzyl)-3-(trifluoromethyl)-1H-pyrazole (62 g, 240 mmol) in THF (200 ml) was added drop wise for 30 min and stirred the contents for another 1 h at −78° C. The reaction mixture was bubbled with dry CO₂ gas for 1½ h. On completion of the reaction, reaction contents were poured into ice water (300 ml) and the aqueous layer was extracted with ethyl acetate (2×200 ml) in basic condition. Aqueous layer was acidified with 6N HCl solution and extracted with ethyl acetate (2×200 ml). Combined organic layer was dried over sodium sulfate and concentrated under reduced pressure to yield the required product as an off white solid (40 g, 55% yield).

Step e: To a solution of 1-(4-methoxybenzyl)-3-(trifluoromethyl)-1H-pyrazole-5-carboxylic acid (50 g, 160 mmol) in dichloromethane (750 ml, 15 times), catalytic amount of DMF was added and cooled to 0° C. Thionyl chloride (99.3 g (61 ml), 0.83 moles, 5 eq) was added drop wise for 30 min at 0° C. Overall reaction mixture was heated to reflux and maintained for 2 hrs. Progress On disappearance of the starting material, dichloromethane and excess of thionyl chloride was distilled off completely. Above prepared acid chloride was dissolved in dichloromethane (500 ml) and added drop wise to aqueous ammonia solution (700 ml) at 0° C. Overall reaction mixture was allowed to stir for 1 hr and the progress of the reaction was monitored by TLC (10% ethyl acetate/hexane, Rf˜0.7). On completion of the reaction, ice cold water (200 ml) was added and the product extracted with ethyl acetate (2×200 ml). Combined organic layer was dried over sodium sulfate and concentrated under reduced pressure to yield the required product as an off white solid (37 g, crude). Crude obtained was directly used for the next step.

Step f: LAH (4.7 g, 120 mmol, 1 eq) was charged into 3N RBF. THF (250 ml) was added at 0° C. Then a solution of step-e product (37 g, 120 mmol) in THF (120 ml) was added drop wise for 30 min at 0° C. and reaction mixture was heated to reflux for 5 h. As the reaction was not moved completely, LAH (2.3 g) was added again and refluxed for another 4 hrs after completion of the reaction, the reaction contents were slowly added to saturated sodium sulfate (1 ltr) solution and filtered over celite and the product extracted with ethyl acetate (2×500 ml). Combined extract was dried over sodium sulfate and concentrated under reduced pressure to obtain the crude product as an off white solid (32.5 g, crude). Crude obtained was directly used for the next step.

Step g: To a solution of (1-(4-methoxybenzyl)-3-(trifluoromethyl)-1H-pyrazol-5-yl)methanamine product ((80 g, 280 mmol) in dichloromethane (600 ml) cooled at 0° C., TEA (28.3 g, 0.28 moles, 1 eq) was added drop wise for 10 min. Then Boc anhydride (61.2 g (62.5 ml), 280 mmol, 1 eq) was added drop wise for 20-30 min at 0° C. Overall reaction mixture stirred for 1 hr at RT. On completion of the reaction, dichloromethane was distilled off completely, residue was taken in ice water (500 ml) and the product extracted with ethyl acetate (2×300 ml). Combined extract was dried over sodium sulfate and concentrated under reduced pressure. Crude obtained was recrystalised from hexane (200 ml) to yield the required product as an off white solid (80 g, 74% yield).

Step h: To a stirred solution of tert-butyl (1-(4-methoxybenzyl)-3-(trifluoromethyl)-1H-pyrazol-5-yl)methylcarbamate (20 g, 52 mmol) in toluene (300 ml, 15 times) cooled to 0° C. was charged aluminum chloride (17.34 g, 129 mmol, 2.5 eq) portion wise for 30 min. Reaction mixture was slowly heated to 50-60° C. and allowed to stir for 2 h at the same temperature. On completion of the reaction, reaction contents were treated with 50 ml dilute HCl, ice cold water (300 ml) was added and extracted with ethyl acetate (2×100 ml). Aqueous layer was basified with 20% sodium hydroxide solution (100 ml) and extracted with ethyl acetate and dried over sodium sulfate and concentrated under reduced pressure to give the crude product as a brown colored solid (4.6 g, crude). The crude obtained was directly used for the next step.

Step i: (3-(Trifluoromethyl)-1H-pyrazol-5-yl)methanamine (0.7 g, 4.2 mmol, 1 eq) was charged in dichloromethane (70 ml) at room temperature, then to that TEA (0.42 g, 4.2 mmol, 1 eq) was added at room temperature and stirred for 10 min and cooled to 0-5° C. (Boc)₂O (0.92 g, 4.2 mmol, 1 eq) was added drop wise to reaction mixture for 30 min and maintained for 3 h at 0-5° C. Progress of the reaction was monitored by the TLC (30% Ethyl acetate/Hexane). On completion of the reaction, dichloromethane was distilled, the residue obtained was treated water (50 ml) and extracted with ethyl acetate (100 ml). The combined organic layer was dried over sodium sulphate, distilled the solvent under vacuum. The obtained crude was purified with column chromatography to yield the required product as a white colored solid (0.5 g, 44% yield).

Step j: tert-Butyl (3-(trifluoromethyl)-1H-pyrazol-5-yl)methylcarbamate (0.3 g, 1.13 mmol, 1 eq) in DMF (3 ml, 10 times) were charged into the 25 ml 3N RB flask at ambient temperature. K₂CO₃ (0.3124 g, 2.264 mmol, 2 eq) was added at same temperature and stirred well for 20 min. Then cyclopropyl methyl bromide (0.22 g, 1.698 mmol, 1.9 eq) was added drop wise to reaction mixture for 10 min. The overall reaction was maintained at ambient temperature for 4 h. Progress of the reaction was monitored by the TLC (30% ethyl acetate/hexane). Cycle propyl methyl bromide (0.5 eq) was added to reaction mixture and maintained for another 12 hrs at ambient temperature. On completion of reaction, reaction contents were poured into ice water (10 ml), and extracted with ethyl acetate (3×10 ml). The combined ethyl acetate layer was washed with water and dried over sodium sulfate and concentrated under reduced pressure, and crude obtained was purified by column chromatography to yield the required product (0.3 g).

Step k: tert-Butyl (1-(cyclopropylmethyl)-3-(trifluoromethyl)-1H-pyrazol-5-yl)methylcarbamate (0.4 g, 1.25 mmol, 1 eq) in dichloromethane (16 ml, 40 times) were charged into 3N RB flask and cooled to 0-5° C. Then dry HCl gas was passed into dichloromethane solution for 30 min. progress of the reaction mass was monitored by TLC (20% ethyl acetate/hexane). On completion of the reaction, dichloromethane was distilled off under vacuum and water (20 ml) was added to reaction mixture and basified to a pH˜10 by 10% NaOH solution, extracted with ethyl acetate (35 ml). Combined ethyl acetate layers were dried over sodium sulphate and distilled off under vacuum to yield the required product as a brown colored liquid (0.240 g, yield 88.8%).

### Synthesis of the Example Compounds

### 1. Preparation of Amides (A=CR^{5b})

General directions for reacting amines of general formula (II) with carboxylic acids of general formula (III) or carboxylic acid derivatives of general formula (IV) to form compounds of general formula (I), wherein A=CR^{5b} (amides), as in scheme 1a (step j09).

### 1.1 Method A:

The acid of general formula (III) (1 equivalent), the amine of general formula (II) (1.2 equivalents) and EDCl (1.2 equivalents) are stirred in DMF (10 mmol of acid/20 ml) for 12 hours at RT and water is subsequently added thereto. The reaction mixture is repeatedly extracted with EE, the aqueous phase is saturated with NaCl and subsequently reextracted with EE. The combined organic phases are washed with 1 N HCl and brine, dried over magnesium sulphate and the solvent is removed under vacuum. The residue is purified by means of flash chromatography (SiO₂, EE/hexane in different ratios such as 1:2) and the product (I) is in this way obtained.

### 1.2 Method B:

The acid of general formula (III) (1 equivalent) and the amine of general formulae (II) (1.1 equivalents) are dissolved in dichloromethane (1 mmol of acid in 6 ml) and mixed with EDCl (1.5 equivalents), HOBt (1.4 equivalents) and triethylamine (3 equivalents) at 0° C. The reaction mixture is stirred for 20 h at room temperature and the crude product is purified by means of column chromatography (SiO₂, n-hexane/EE in different ratios such as 2:1) and (I) is in this way obtained.

### 1.3 Method C:

The acid of general formula (III) (1 equivalent) is first mixed with a chlorinating agent, preferably with thionyl chloride and the mixture obtained in this way is boiled under reflux and the acid (III) is in this way converted into the corresponding acid chloride (IV). The amine of general formulae (II) (1.1 equivalents) is dissolved in dichloromethane (1 mmol of acid in 6 ml) and mixed with triethylamine (3 equivalents) at 0° C. The reaction mixture is stirred for 20 h at room temperature and the crude product is purified by means of column chromatography (SiO₂, n-hexane/EE in different ratios such as 2:1) and (I) is in this way obtained.

### 1.4 Method D:

The phenyl ester (IVa) obtained (1 equivalent) and the corresponding amine (II) (1.1 equivalents) are dissolved in THF (10 mmol of the reaction mixture in 120 ml) and stirred for 16 h at room temperature after addition of DBU (1.5 equivalents). After removal of the solvent under vacuum, the residue obtained is purified by means of flash chromatography (SiO₂, EE/hexane in different ratios such as 1:1) and (I) is in this way obtained.

The following example compounds I-15 and 18 were obtained by one of the methods disclosed above.

| | |
|---|---|
| 1 | 2-(1-methyl-1H-indol-3-yl)-N-((1-phenyl-3-(trifluoromethyl)-1H- |
| | pyrazol-5-yl)methyl)propanamide |
| 2 | N-((3-tert-butyl-1-(3-chlorophenyl)-1H-pyrazol-5-yl)methyl)- |
| | 2-(1-methyl-1H-indol-3-yl)propanamide |
| 3 | N-((1-(3-methoxyphenyl)-3-(trifluoromethyl)-1H-pyrazol-5- |
| | yl)methyl)-2-(1-methyl-1H-indol-3-yl)propanamide |
| 4 | N-((1-cyclopentyl-3-(trifluoromethyl)-1H-pyrazol-5-yl)methyl)-2-(5- |
| | hydroxy-1-methyl-1H-indol-3-yl)propanamide |
| 5 | 2-(5-hydroxy-1-methyl-1H-indol-3-yl)-N-((1-m-tolyl-3- |
| | (trifluoromethyl)-1H-pyrazol-5-yl)methyl)propanamide |
| 6 | N-((3-tert-butyl-1-(pyridin-2-yl)-1H-pyrazol-5-yl)methyl)-2-(5- |
| | hydroxy-1-methyl-1H-indol-3-yl)propanamide |
| 7 | N-((1-(3-chlorophenyl)-4-methyl-3-(trifluoromethyl)-1H-pyrazol-5- |
| | yl)methyl)-2-(5-hydroxy-1-methyl-1H-indol-3-yl)propanamide |
| 8 | N-((1-(3-chlorophenyl)-3-(trifluoromethyl)-1H-pyrazol-5-yl)methyl)- |
| | 2-(1-methyl-1H-indol-3-yl)propanamide |
| 9 | N-((1-(3-chlorophenyl)-3-(trifluoromethyl)-1H-pyrazol-5-yl)methyl)- |
| | 2-(5-hydroxy-1-methyl-1H-indol-3-yl)acetamide |
| 10 | N-((1-(3-chlorophenyl)-3-(trifluoromethyl)-1H-pyrazol-5-yl)methyl)- |
| | 2-(5-hydroxy-1-methyl-1H-indol-3-yl)propanamide |
| 11 | N-((3-tert-butyl-1-(3-chloro-4-fluorophenyl)-1H-pyrazol-5- |
| | yl)methyl)-2-(5-hydroxy-1-methyl-1H-indol-3-yl)propanamide |
| 12 | N-((1-(3-chlorophenyl)-3-cyclopropyl-1H-pyrazol-5-yl)methyl)- |
| | 2-(5-hydroxy-1-methyl-1H-indol-3-yl)propanamide |
| 13 | N-((3-tert-butyl-1-(3-chlorophenyl)-1H-pyrazol-5-yl)methyl)-2- |
| | (5-methoxy-1-methyl-1H-indol-3-yl)acetamide |
| 14 | N-((3-tert-butyl-1-(3-chlorophenyl)-1H-pyrazol-5-yl)methyl)-2- |
| | (5-methoxy-1-methyl-1H-indol-3-yl)propanamide |
| 15 | N-((1-(cyclopropylmethyl)-3-(trifluoromethyl)-1H-pyrazol-5- |
| | yl)methyl)-2-(1-methyl-1H-indol-3-yl)propanamide |
| 18 | N-((3-tert-butyl-1-(3-chlorophenyl)-1H-1,2,4-triazol-5-yl)methyl)- |
| | 2-(5-hydroxy-1-methyl-1H-indol-3-yl)acetamide |

The following example compounds 19-33 and 35 can be obtained by one of the methods disclosed above.

| | |
|---|---|
| 19 | N-((3-tert-butyl-1-methyl-1H-pyrazol-5-yl)methyl)-2-(5-hydroxy-1- |
| | methyl-1H-indol-3-yl)propanamide |
| 20 | N-((3-tert-butyl-1-hexyl-1H-pyrazol-5-yl)methyl)-2-(5-hydroxy-1- |
| | methyl-1H-indol-3-yl)propanamide |
| 21 | N-((1-cyclohexyl-3-(trifluoromethyl)-1H-pyrazol-5-yl)methyl)-2-(5- |
| | hydroxy-1-methyl-1H-indol-3-yl)propanamide |
| 22 | 2-(5-hydroxy-1-methyl-1H-indol-3-yl)-N-((1-(tetrahydro-2H-pyran-4- |
| | yl)-3-(trifluoromethyl)-1H-pyrazol-5-yl)methyl)propanamide |
| 23 | 2-(5-hydroxy-1-methyl-1H-indol-3-yl)-N-((1-(oxetan-3-yl)-3- |
| | (trifluoromethyl)-1H-pyrazol-5-yl)methyl)propanamide |
| 24 | N-((3-tert-butyl-1-(3-fluorophenyl)-1H-pyrazol-5-yl)methyl)-2- |
| | (5-hydroxy-1-methyl-1H-indol-3-yl)propanamide |
| 25 | N-((3-tert-butyl-1-(4-methoxyphenyl)-1H-pyrazol-5-yl)methyl)- |
| | 2-(5-hydroxy-1-methyl-1H-indol-3-yl)propanamide |
| 26 | N-((3-tert-butyl-1-(3-chloro-4-fluorophenyl)-1H-pyrazol-5- |
| | yl)methyl)-2-(5-hydroxy-1-methyl-1H-indol-3-yl)propanamide |
| 27 | 2-(5-hydroxy-1-methyl-1H-indol-3-yl)-N-((1-(4-methoxybenzyl)-3- |
| | (trifluoromethyl)-1H-pyrazol-5-yl)methyl)propanamide |
| 28 | 2-(5-hydroxy-1-methyl-1H-indol-3-yl)-N-((1-phenyl-3- |
| | (trifluoromethyl)-1H-pyrazol-5-yl)methyl)propanamide |
| 29 | N-((1-(4-fluorophenyl)-3-(trifluoromethyl)-1H-pyrazol-5- |
| | yl)methyl)-2-(5-hydroxy-1-methyl-1H-indol-3-yl)propanamide |
| 30 | 2-(5-hydroxy-1-methyl-1H-indol-3-yl)-N-((1-(3-hydroxyphenyl)-3- |
| | (trifluoromethyl)-1H-pyrazol-5-yl)methyl)propanamide |
| 31 | 2-(5-hydroxy-1-methyl-1H-indol-3-yl)-N-((1-(3-isopropylphenyl)-3- |
| | (trifluoromethyl)-1H-pyrazol-5-yl)methyl)propanamide |
| 32 | 2-(5-hydroxy-1-methyl-1H-indol-3-yl)-N-((1-(pyridin-3-yl)-3- |
| | (trifluoromethyl)-1H-pyrazol-5-yl)methyl)propanamide |
| 33 | 2-(5-hydroxy-1-methyl-1H-indol-3-yl)-N-((1-(pyrimidin-2-yl)-3- |
| | (trifluoromethyl)-1H-pyrazol-5-yl)methyl)propanamide |
| 35 | N-((1-(3-chlorophenyl)-3-(trifluoromethyl)-1H-pyrazol-5-yl)methyl)- |
| | 2-(1,5-dimethyl-1H-indol-3-yl)propanamide |

### 2. Preparation of Ureas (A=N)

General directions for reacting amines of general formula (II) or (VI) with phenyl chloroformate to form compounds of formula (V) or (Via) (step j07 and step j10, respectively) and subsequent reaction of compounds of formula (V) with amines of general formula (VI) or of compounds of formula (VIa) with amines of general formula (II) to form compounds of general formula (I), wherein A=N, as in scheme 1a and 1c (step j08 and step j11, respectively):

Step j07/step j10: The amine of general formula (II) or (VI) (1 equivalent) is placed in dichloromethane (10 mmol of amine in 70 ml) and phenyl chloroformate (1.1 equivalents) is added thereto at room temperature and the mixture is stirred for 30 min. After removal of the solvent under vacuum, the residue is purified by means of flash chromatography (SiO₂, diethyl ether/hexane in different ratios such as 1:2) and (V) or (VIa) is in this way obtained.

Step j08/step j11: The carbamic acid phenyl ester (V) or (Via) obtained (1 equivalent) and the corresponding amine (VI) or (II) (1.1 equivalents) are dissolved in THF (10 mmol of the reaction mixture in 120 ml) and stirred for 16 h at room temperature after addition of DBU (1.5 equivalents). After removal of the solvent under vacuum, the residue obtained is purified by means of flash chromatography (SiO₂, EE/hexane in different ratios such as 1:1) and (I) is in this way obtained.

The following example compounds 16 and 17 were obtained according to the methods disclosed above.

| | |
|---|---|
| 16 | 1-((1-(3-chlorophenyl)-3-(trifluoromethyl)-1H-pyrazol-5-yl)methyl)- |
| | 3-(1H-indazol-3-yl)urea |
| 17 | 1-((1-(3-chlorophenyl)-3-(trifluoromethyl)-1H-pyrazol-5-yl)methyl)- |
| | 3-(1-methyl-1H-pyrazolo[3,4-b]pyridin-3-yl)urea |

The following example compounds 34, 36-38 and 40-49 can be obtained according to the methods disclosed above.

| | |
|---|---|
| 34 | 1-((1-(3-chlorophenyl)-3-(trifluoromethyl)-1H-pyrazol-5-yl)methyl)- |
| | 3-(1-methyl-1H-pyrrolo[2,3-c]pyridin-3-yl)urea |
| 36 | 1-((1-(3-chlorophenyl)-3-(trifluoromethyl)-1H-pyrazol-5-yl)methyl)- |
| | 3-(5-fluoro-1-methyl-1H-indol-3-yl)urea |
| 37 | 1-((1-(3-chlorophenyl)-3-(trifluoromethyl)-1H-pyrazol-5-yl)methyl)- |
| | 3-(7-methyl-7H-pyrrolo[2,3-d]pyrimidin-5-yl)urea |
| 38 | 1-((1-(3-chlorophenyl)-3-(trifluoromethyl)-1H-pyrazol-5-yl)methyl)- |
| | 3-(5-(dimethylamino)-1-methyl-1H-indol-3-yl)urea |
| 40 | 1-((1-(3-chlorophenyl)-3-(trifluoromethyl)-1H-pyrazol-5-yl)methyl)- |
| | 3-(5-hydroxybenzo[d]oxazol-2-yl)urea |
| 41 | 1-((1-(3-chlorophenyl)-3-(trifluoromethyl)-1H-pyrazol-5-yl)methyl)- |
| | 3-(6-hydroxybenzo[d]oxazol-2-yl)urea |
| 42 | 1-((1-(3-chlorophenyl)-3-(trifluoromethyl)-1H-pyrazol-5-yl)methyl)- |
| | 3-(4-hydroxybenzo[d]oxazol-2-yl)urea |
| 43 | 1-(1H-benzo[d]imidazol-2-yl)-3-((1-(3-chlorophenyl)-3- |
| | (trifluoromethyl)-1H-pyrazol-5-yl)methyl)urea |
| 44 | 1-(1H-benzo[d]imidazol-2-yl)-3-((1-(3-chlorophenyl)-3- |
| | (trifluoromethyl)-1H-pyrazol-5-yl)methyl)urea |
| 45 | 1-((1-(3-chlorophenyl)-3-(trifluoromethyl)-1H-pyrazol-5-yl)methyl)- |
| | 3-(1-methyl-1H-benzo[d]imidazol-2-yl)urea |
| 46 | 1-(6-chloro-1H-benzo[d]imidazol-2-yl)-3-((1-(3- |
| | chlorophenyl)-3-(trifluoromethyl)-1H-pyrazol-5-yl)methyl)urea |
| 47 | 1-(5-chlorobenzo[d]oxazol-2-yl)-3-((1-(3-chlorophenyl)- |
| | 3-(trifluoromethyl)-1H-pyrazol-5-yl)methyl)urea |
| 48 | 1-((1-(3-chlorophenyl)-3-(trifluoromethyl)-1H-pyrazol-5-yl)methyl)- |
| | 3-(6-methoxybenzo[d]thiazol-2-yl)urea |
| 49 | 1-((1-(3-chlorophenyl)-3-(trifluoromethyl)-1H-pyrazol-5-yl)methyl)- |
| | 3-(6-(methylsulfonyl)benzo[d]thiazol-2-yl)urea |

Mass spectrometric data are cited hereinafter by way of example for the following example compounds:

| Example | |
|---|---|
| compound | [M + H] |
| 1 | 427.0 |
| 2 | 449.1 |
| 3 | 457.1 |
| 4 | 446.1 |
| 5 | 457.2 |
| 6 | 432.13 |
| 7 | 491.0 |
| 8 | 460.9 |
| 9 | 463.0 |
| 10 | 477.3 |
| 11 | 483.1 |
| 12 | 449.2 |
| 13 | 465.0 |
| 14 | 479.0 |
| 15 | 405.1 |
| 16 | 435.0 |
| 17 | 450.1 |
| 18 | 452.1 |

### Pharmacological Data

The affinity of the compounds according to the invention for the vanilloid receptor 1 (VR1/TRPV1 receptor) was determined as described hereinbefore (pharmacological methods I and II respectively). The compounds according to the invention of the above-indicated formula (I) display outstanding affinity to the VR1/TRPV1 receptor (Table 1.).

In Table 1 the abbreviations below have the following meanings:

Cap=capsaicin
AG=agonist
pAG=partial agonist
pH=after pH stimulus
NADA=N-arachidonoyl dopamine
NE=no effect
FTm=formalin test carried out on mice
CClm=Bennet model in mice

The value after the “@” symbol indicates the concentration at which the inhibition (as a percentage) was respectively determined.

TABLE 1

| Compound | | | IC₅₀ (human |
|---|---|---|---|
| according | Kᵢ (mouse) | Kᵢ (human being) | being) |
| to Example | [nM] Cap | [nM] Cap | [nM], 45° C. |
| 1 | | 42.1 | |
| 2 | | 33.6 | |
| 3 | | 49.8 | |
| 4 | | 4.8 | 92 |
| 5 | 38.5 | 0.6 | |
| 6 | | 56.8 | |
| 7 | | 0.4 | 36% @ 2.5 μM |
| 8 | | 6.4 | 924 |
| 9 | | 0.25 | 1725 |
| 10 | 4.8 | 0.4 | |
| 11 | | 0.7 | 39 |
| 12 | | 1.1 | 95.8 |
| 13 | | 33.3 | |
| 14 | | 48.1 | |
| 15 | | 14% @ 5 μM | |
| 16 | | 24% @ 5 μM | |
| 17 | | 30% @ 5 μM; | |
| | | 4% @ 1 μM | |
| 18 | | 70.9 | |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A compound corresponding to the formula (I): 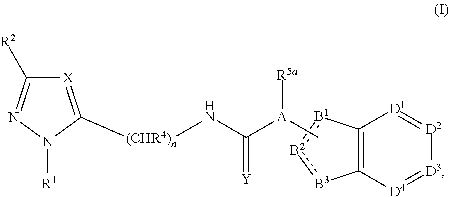
wherein
- - - in each case represents the presence of precisely one double bond between B¹ and B² or between B² and B³;
X represents CR³ or N, wherein
R³ represents H; C₁₋₁₀ alkyl, saturated or unsaturated, branched or unbranched, unsubstituted or mono- or polysubstituted;
A represents N or CR^{5b};
n represents 1, 2, 3 or 4;
Y represents O or S;
R⁰ represents C₁₋₁₀ alkyl, saturated or unsaturated, branched or unbranched, unsubstituted or mono- or polysubstituted; C₃₋₁₀ cycloalkyl or heterocyclyl, respectively saturated or unsaturated, unsubstituted or mono- or polysubstituted; aryl or heteroaryl, respectively unsubstituted or mono- or polysubstituted; C₃₋₁₀ cycloalkyl or heterocyclyl bridged via C₁₋₈ alkyl, respectively saturated or unsaturated, unsubstituted or mono- or polysubstituted, wherein the alkyl chain can be respectively branched or unbranched, saturated or unsaturated, unsubstituted, mono- or polysubstituted; or aryl or heteroaryl bridged via C₁₋₈ alkyl, respectively unsubstituted or mono- or polysubstituted, wherein the alkyl chain can be respectively branched or unbranched, saturated or unsaturated, unsubstituted, mono- or polysubstituted;
R¹ represents C₁₋₁₀ alkyl, saturated or unsaturated, branched or unbranched, unsubstituted or mono- or polysubstituted; C₃₋₁₀ cycloalkyl or heterocyclyl, respectively saturated or unsaturated, unsubstituted or mono- or polysubstituted; aryl or heteroaryl, respectively unsubstituted or mono- or polysubstituted; C₃₋₁₀ cycloalkyl or heterocyclyl bridged via C₁₋₈ alkyl, respectively saturated or unsaturated, unsubstituted or mono- or polysubstituted, wherein the alkyl chain can be respectively branched or unbranched, saturated or unsaturated, unsubstituted, mono- or polysubstituted; or aryl or heteroaryl bridged via C₁₋₈ alkyl, respectively unsubstituted or mono- or polysubstituted, wherein the alkyl chain can be respectively branched or unbranched, saturated or unsaturated, unsubstituted, mono- or polysubstituted; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰ (S(═O)₂—R⁰); or SCl₃;
R² represents H; R⁰; F; Cl; Br; I; CN; NO₂; OH; SH; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅;
R⁴ represents H; F; Cl; Br; I; OH; C₁₋₁₀ alkyl, saturated or unsaturated, branched or unbranched, unsubstituted or mono- or polysubstituted;
R^{5a} represents H; OH; C₁₋₁₀ alkyl, saturated or unsaturated, branched or unbranched, unsubstituted or mono- or polysubstituted; and
R^{5b} represents H or R⁰; or
R^{5a} and R^{5b} together with the carbon atom connecting them form a C₃₋₁₀ cycloalkyl group or a heterocyclyl group, respectively saturated or unsaturated, unsubstituted or mono- or polysubstituted;
B¹ represents C, CH, N, NR⁶, O or S;
B² represents C, CH, N, NR⁷, O or S;
B³ represents C, CH, N, NR⁸, O or S;
wherein 1 or 2 of the variables B¹, B² and B³ represent one of the aforementioned heteroatoms or heteroatom groups;
D¹ represents N or CR⁹;
D² represents N or CR¹⁰;
D³ represents N or CR¹¹;
D⁴ represents N or CR¹²;
wherein 0, 1 or 2 of the variables D¹, D², D³ and D⁴ represent N;
R⁶, R⁷ and R⁸ each independently represent H or C₁₋₄ alkyl, saturated, branched or unbranched, unsubstituted or mono- or polysubstituted;
R⁹, R¹⁰, R¹¹ and R¹² each independently represent H; F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂, NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
in which “substituted alkyl”, “substituted heterocyclyl” and “substituted cycloalkyl” relate, with respect to the corresponding residues, to the replacement of one or more hydrogen atoms each independently of one another by F; Cl; Br; I; NO₂; CN; ═O; ═NH; ═N(OH); ═C(NH₂)₂;
CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂, NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂; and
in which “aryl substituted” and “heteroaryl substituted” relate, with respect to the corresponding residues, to the substitution of one or more hydrogen atoms each independently of one another by F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂, NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
in the form of a free compound or a salt with a physiologically compatible acid or base; a tautomer; an N-oxide; a racemate; an individual enantiomer or diastereomer, or a mixture of enantiomers or diastereomers.

2. A compound according to claim 1, wherein R¹ represents a substructure of formula (T1) 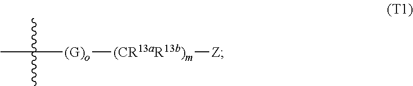
wherein
G represents C(═O), O, S, S(═O)₂, NH—C(═O) or NR¹⁴; wherein
R¹⁴ represents H; C₁₋₈ alkyl or S(═O)₂—C₁₋₈ alkyl, in which C₁₋₈ alkyl can be respectively saturated or unsaturated, branched or unbranched, unsubstituted or mono- or polysubstituted with one or more substituents each selected independently from the group consisting of F, Cl, Br, I, OH, O—C₁₋₄ alkyl, OCF₃, NH₂, NH—C₁₋₄ alkyl and N(C₁₋₄ alkyl)₂;
o represents 0 or 1;
R^{13a} and R^{13b} each independently represent H; F; Cl; Br; I; NO₂; CF₃; CN; OH; OCF₃; NH₂; C₁₋₄ alkyl, O—C₁₋₄ alkyl, NH—C₁₋₄ alkyl, N(C₁₋₄ alkyl)₂, in which C₁₋₄ alkyl can be respectively saturated or unsaturated, branched or unbranched, unsubstituted or mono- or polysubstituted with one or more substituents each selected independently from the group consisting of F, Cl, Br, I, O—C₁₋₄ alkyl, OH and OCF₃,
with the proviso that if R^{13a} and R^{13b} are bound to the same carbon atom, only one of R^{13a} and R^{13b} can represent OH; OCF₃; NH₂; O—C₁₋₄ alkyl, NH—C₁₋₄ alkyl or N(C₁₋₄ alkyl)₂;
m represents 0, 1, 2, 3 or 4; and
Z represents C₁₋₄ alkyl, saturated or unsaturated, branched or unbranched, unsubstituted or mono- or polysubstituted with one or more substituents each selected independently from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ and S(═O)₂OH; C₃₋₁₀ cycloalkyl or heterocyclyl, respectively saturated or unsaturated, unsubstituted or mono- or polysubstituted with one or more substituents each selected independently from the group consisting of F, Cl, Br, I, NO₂, CN, OH, O—C₁₋₄ alkyl, OCF₃, C₁₋₄ alkyl, C(═O)—OH, CF₃, SH, S—C₁₋₄ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; aryl or heteroaryl, respectively unsubstituted or mono- or polysubstituted with one or more substituents each selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

3. A compound according to claim 2, wherein
G represents C(═O), O, S, S(═O)₂, NH—C(═O) or NR¹⁴, wherein
R¹⁴ represents H; methyl; ethyl; n-propyl; isopropyl; n-butyl; sec.-butyl; tert.-butyl; S(═O)₂-methyl; S(═O)₂-ethyl;
o represents 0 or 1;
R^{13a} and R^{13b} each independently represent H; F; Cl; Br; I; NO₂; CF₃; CN; methyl; ethyl; n-propyl; isopropyl; n-butyl; sec.-butyl; tert.-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂; or N(methyl)(ethyl);
with the proviso that if R^{13a} and R^{13b} are bound to the same carbon atom, only one of R^{13a} and R^{13b} can represent OH; OCF₃; O-methyl; O-ethyl; O—(CH₂)₂—O—CH₃; O—(CH₂)₂—OH; NH₂; NH-methyl; N(methyl)₂; NH-ethyl; N(ethyl)₂; or N(methyl)(ethyl);
m represents 0, 1 or 2; and
Z represents C₁₋₄ alkyl, saturated or unsaturated, branched or unbranched, unsubstituted or mono- or polysubstituted with one or more substituents each selected independently from the group consisting of F, Cl, Br, I, OH, ═O, O—C₁₋₄ alkyl, OCF₃, C(═O)—OH and CF₃; phenyl, naphthyl, furyl, pyridyl or thienyl, respectively unsubstituted or mono- or polysubstituted with one or more substituents each selected independently from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₈ alkyl, SCF₃, benzyl and phenyl, wherein benzyl and phenyl can be respectively unsubstituted or mono- or polysubstituted with one or more substituents selected independently from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl and SCF₃; C₃₋₁₀ cycloalkyl or heterocyclyl, respectively saturated or unsaturated, unsubstituted or mono- or polysubstituted with one or more substituents each selected independently from the group consisting of F, Cl, Br, I, CN, OH, O—C₁₋₄ alkyl, OCF₃, C₁₋₄ alkyl, CF₃, benzyl, phenyl and pyridyl, wherein benzyl, phenyl and pyridyl can be respectively unsubstituted or mono- or polysubstituted with one or more substituents selected independently from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl and SCF₃.

4. A compound according to claim 1, wherein R² represents H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; C₁₋₁₀ alkyl, saturated or unsaturated, branched or unbranched, unsubstituted or mono- or polysubstituted with one or more substituents each selected independently from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃S(═O)₂OH, benzyl, phenyl, pyridyl and thienyl, wherein benzyl, phenyl, pyridyl, thienyl can be respectively unsubstituted or mono- or polysubstituted with one or more substituents selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; C₃₋₁₀ cycloalkyl or heterocyclyl, respectively saturated or unsaturated, unsubstituted or mono- or polysubstituted with one or more substituents selected independently from the group consisting of F, Cl, Br, I, OH, ═O, C₁₋₄ alkyl, O—C₁₋₄ alkyl, OCF₃, C(═O)—OH and CF₃; or C₃₋₁₀ cycloalkyl or heterocyclyl bridged via C₁₋₈ alkyl, respectively saturated or unsaturated, unsubstituted or mono- or polysubstituted with one or more substituents each selected independently from the group consisting of F, Cl, Br, I, OH, ═O, C₁₋₄ alkyl, O—C₁₋₄ alkyl, OCF₃, C(═O)—OH and CF₃, wherein the alkyl chain can be respectively branched or unbranched, saturated or unsaturated, unsubstituted, mono- or polysubstituted with one or more substituents each selected independently from the group consisting of F, Cl, Br, I, OH, ═O and O—C₁₋₄ alkyl; aryl or heteroaryl, respectively unsubstituted or mono- or polysubstituted with one or more substituents each selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₈ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or aryl or heteroaryl bridged via C₁₋₈ alkyl, respectively unsubstituted or mono- or polysubstituted with one or more substituents each selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₈ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH, wherein the alkyl chain can be respectively branched or unbranched, saturated or unsaturated, unsubstituted, mono- or polysubstituted with one or more substituents each selected independently from the group consisting of F, Cl, Br, I, OH, ═O and O—C₁₋₄ alkyl.

5. A compound according to claim 1, wherein R⁴ represents H or C₁₋₁₀ alkyl, saturated or unsaturated, branched or unbranched, unsubstituted or mono- or polysubstituted with one or more substituents each selected independently from the group consisting of F, Cl, Br; I, OH and O—C₁₋₄ alkyl.

6. A compound according to claim 1, wherein
R^{5a} represents H; OH; C₁₋₁₀ alkyl, saturated or unsaturated, branched or unbranched, unsubstituted or mono- or polysubstituted with one or more substituents each selected independently from the group consisting of F, Cl, Br; I, OH and O—C₁₋₄ alkyl; and
R^{5b} represents H; C₁₋₁₀ alkyl, saturated or unsaturated, branched or unbranched, unsubstituted or mono- or polysubstituted with one or more substituents each selected independently from the group consisting of F, Cl, Br, I, OH and O—C₁₋₄ alkyl; C₃₋₁₀ cycloalkyl or heterocyclyl, respectively saturated or unsaturated, unsubstituted or mono- or polysubstituted with one or more substituents each selected independently from the group consisting of F, Cl, Br, I, OH, ═O and O—C₁₋₄ alkyl; or C₃₋₁₀ cycloalkyl or heterocyclyl bridged via C₁₋₈ alkyl, respectively saturated or unsaturated, unsubstituted or mono- or polysubstituted with one or more substituents each selected independently from the group consisting of F, Cl, Br, I, OH, ═O and O—C₁₋₄ alkyl, wherein the alkyl chain can be respectively branched or unbranched, saturated or unsaturated, unsubstituted, mono- or polysubstituted with one or more substituents each selected independently from the group consisting of F, Cl, Br, I, OH, ═O and O—C₁₋₄ alkyl; or aryl, heteroaryl, respectively unsubstituted or mono- or polysubstituted with one or more substituents each selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl; or aryl or heteroaryl bridged via C₁₋₈ alkyl, respectively unsubstituted or mono- or polysubstituted with one or more substituents each selected independently from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl, wherein the alkyl chain can be respectively branched or unbranched, saturated or unsaturated, unsubstituted, mono- or polysubstituted with one or more substituents each selected independently from the group consisting of F, Cl, Br, I, OH, ═O and O—C₁₋₄ alkyl; or
R^{5a} and R^{5b} together with the carbon atom connecting them form a C₃₋₁₀ cycloalkyl group or a heterocyclyl group, respectively saturated or unsaturated, unsubstituted or mono- or polysubstituted with one or more substituents each selected independently from the group consisting of F, Cl, Br, I, OH, ═O and O—C₁₋₄ alkyl.

7. A compound according to claim 1, wherein
R^{5a} represents H or CH₃, if A represents N; or
R^{5a} represents H or CH₃, if A represents CR^{5b}, wherein
R^{5b} represents H; or C₁₋₄ alkyl, saturated or unsaturated, branched or unbranched, unsubstituted; C₃₋₁₀ cycloalkyl, saturated or unsaturated, unsubstituted; or phenyl or benzyl, in each case unsubstituted or mono- or polysubstituted with one or more substituents each selected independently from the group consisting of F, Cl, Br, I, CF₃, O—C₁₋₄ alkyl, OCF₃ and C₁₋₄ alkyl; or
R^{5a} and R^{5b} together with the carbon atom connecting them form a C₃₋₁₀ cycloalkyl group, saturated or unsaturated, unsubstituted or mono- or polysubstituted with one or more substituents each selected independently from the group consisting of F, Cl, Br, I, OH, ═O and O—C₁₋₄ alkyl.

8. A compound according to claim 1, wherein the partial structure (T2) 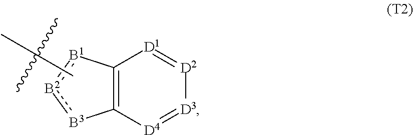
is selected from the group consisting of:

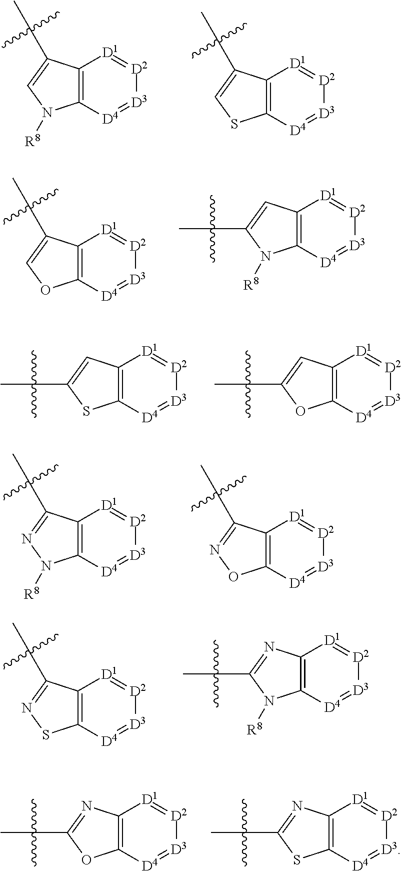

9. A compound according to claim 8, wherein the partial structure (T2) is selected from the group consisting of: 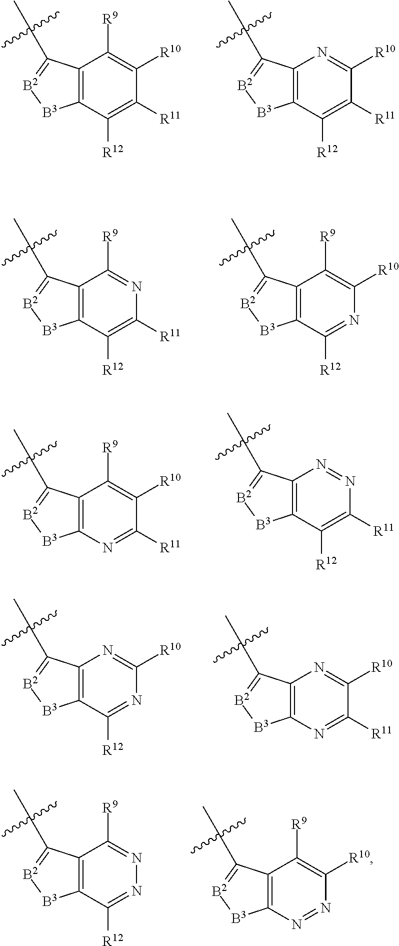
wherein in each case independently
B² represents C or N, and
B³ represents NR⁸, O or S;
or wherein the partial structure (T2) is selected from the group consisting of: 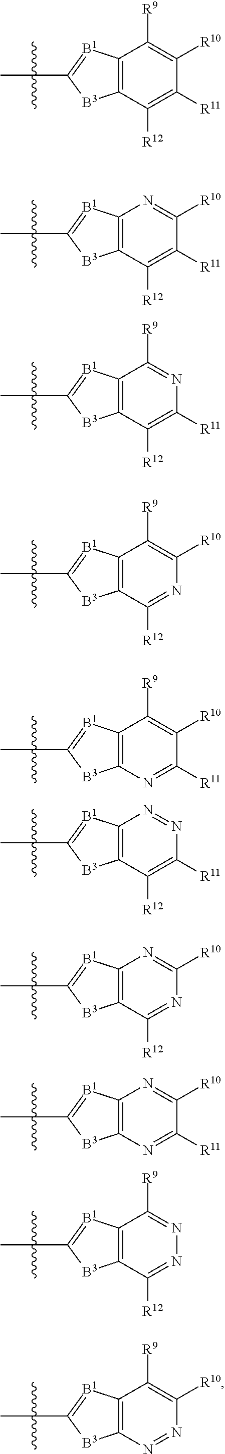
wherein in each case independently
B¹ represents C or N, and
B³ represents NR⁸, O or S.

10. A compound according to claim 1, wherein R⁶, R⁷ and R⁸ each independently represent H, methyl or ethyl.

11. A compound according to claim 1, wherein R⁹, R¹⁰, R¹¹ and R¹² are each independently selected from the group consisting of:
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C₁₋₁₀ alkyl, C₁₋₁₀ alkyl-O—C₁₋₁₀ alkyl, C(═O)—NH—C₁₋₁₀ alkyl, O—C₁₋₁₀ alkyl, NH(C₁₋₁₀ alkyl), N(C₁₋₁₀ alkyl)₂, NH—C(═O)—C₁₋₁₀ alkyl, N(C₁₋₁₀ alkyl)-C(═O)—C₁₋₁₀ alkyl, NH—S(═O)₂—C₁₋₁₀ alkyl, S—C₁₋₁₀ alkyl, SO₂—C₁₋₁₀ alkyl, SO₂—NH(C₁₋₁₀ alkyl), SO₂—N(C₁₋₁₀ alkyl)₂, in which C₁₋₁₀ alkyl can be respectively saturated or unsaturated, branched or unbranched, unsubstituted or mono- or polysubstituted with one or more substituents selected independently from the group consisting of F, Cl, Br, I, NO₂, CN, OH, O—C₁₋₄ alkyl, OCF₃, CF₃, NH₂, NH(C₁₋₄ alkyl), N(C₁₋₄ alkyl)₂, NH—S(═O)₂—C₁₋₄ alkyl, N(C₁₋₄ alkyl)-S(═O)₂—C₁₋₄ alkyl, SH, S—C₁₋₄ alkyl, S(═O)₂—C₁₋₄ alkyl and SCF₃;
C₃₋₁₀ cycloalkyl, heterocyclyl or C₃₋₁₀ cycloalkyl or heterocyclyl bridged via C₁₋₈ alkyl, respectively saturated or unsaturated, unsubstituted or mono- or polysubstituted with one or more substituents selected independently from the group consisting of F, Cl, Br, I, NO₂, CN, OH, O—C₁₋₄ alkyl, OCF₃, CF₃, C₁₋₄ alkyl, NH₂, NH(C₁₋₄ alkyl), N(C₁₋₄ alkyl)₂, NH—S(═O)₂—C₁₋₄ alkyl, N(C₁₋₄ alkyl)-S(═O)₂—C₁₋₄ alkyl, SH, S—C₁₋₄ alkyl, S(═O)₂—C₁₋₄ alkyl and SCF₃, and wherein if appropriate the alkyl chain can be respectively branched or unbranched, saturated or unsaturated, unsubstituted, mono- or polysubstituted with one or more substituents each selected independently from the group consisting of F, Cl, Br; I, OH and O—C₁₋₄ alkyl; and aryl, heteroaryl, C(═O)—NH-aryl, C(═O)—NH-heteroaryl, NH—C(═O)-aryl, NH(C═O)-heteroaryl, NH(aryl), NH(heteroaryl), N(aryl)₂, N(heteroaryl)₂ or aryl or heteroaryl bridged via C₁₋₈ alkyl, respectively unsubstituted or mono- or polysubstituted with one or more substituents selected independently from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl and SCF₃, and wherein the alkyl chain optionally may be respectively branched or unbranched, saturated or unsaturated, unsubstituted, mono- or polysubstituted with one or more substituents each selected independently from the group consisting of F, Cl, Br; I, OH and O—C₁₋₄ alkyl.

12. A compound according to claim 1, corresponding to formula (I′) 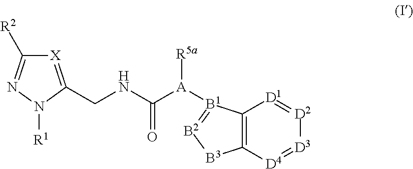
wherein
R¹ represents the partial structure (T1) 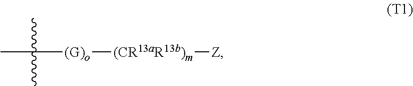
wherein
G represents C(═O), O, S, S(═O)₂, NH—C(═O) or NR¹⁴, wherein
R¹⁴ represents H; methyl; ethyl; n-propyl; isopropyl; n-butyl; sec.-butyl; tert.-butyl; S(═O)₂-methyl; or S(═O)₂-ethyl;
o represents 0 or 1;
R^{13a} and R^{13b} each independently represent H; F; Cl; Br; I; methyl; ethyl; n-propyl; isopropyl; n-butyl; sec.-butyl; tert.-butyl; OH; O-methyl; O-ethyl,
with the proviso that if R^{13a} and R^{13b} are bound to the same carbon atom, only one of R^{13a} and R^{13b} can represent OH; O-methyl; O-ethyl;
m represents 0, 1 or 2; and
Z represents
C₁₋₄ alkyl, saturated or unsaturated, branched or unbranched, unsubstituted or mono- or polysubstituted with one or more substituents each selected independently from the group consisting of F, Cl, Br, I, OH, O—C₁₋₄ alkyl, OCF₃, and CF₃;
C₃₋₁₀ cycloalkyl, saturated or unsaturated, unsubstituted or mono- or polysubstituted with one or more substituents each selected independently from the group consisting of F, Cl, Br, I, OH, O—C₁₋₄ alkyl, OCF₃, C₁₋₄ alkyl, CF₃, benzyl and phenyl, wherein benzyl and phenyl can be respectively unsubstituted or mono- or polysubstituted with one or more substituents selected independently from the group consisting of F, Cl, Br, I, OH, O—C₁₋₄ alkyl, OCF₃, C₁₋₄ alkyl, CF₃, and SCF₃;
morpholinyl, thiomorpholinyl, piperidinyl, pyrrolidinyl, 4-methylpiperazinyl, piperazinyl, respectively unsubstituted or mono- or polysubstituted with one or more substituents each selected independently from the group consisting of F, Cl, Br, I, OH, O—C₁₋₄ alkyl, OCF₃, C₁₋₄ alkyl, CF₃, benzyl and phenyl, wherein benzyl and phenyl can be respectively unsubstituted or mono- or polysubstituted with one or more substituents selected independently from the group consisting of F, Cl, Br, I, OH, O—C₁₋₄ alkyl, OCF₃, C₁₋₄ alkyl, CF₃ and SCF₃; or
phenyl, naphthyl, pyridyl or thienyl, respectively unsubstituted or mono- or polysubstituted with one or more substituents each selected independently from the group consisting of F, Cl, Br, I, CN, OH, C₁₋₄ alkyl, O—C₁₋₄ alkyl, OCF₃, C₁₋₄ alkyl, CF₃, SH, S—C₁₋₄ alkyl, SCF₃, benzyl and phenyl, wherein benzyl and phenyl can be respectively unsubstituted or mono- or polysubstituted with one or more substituents selected independently from the group consisting of F, Cl, Br, I, OH, O—C₁₋₄ alkyl, OCF₃, C₁₋₄ alkyl, CF₃ and SCF₃;
R² represents tert-butyl, CF₃ or cyclopropyl;
X represents CR³ or N, wherein
R³ represents H or C₁₋₄ alkyl, saturated, branched or unbranched, unsubstituted;
A represents N or CR^{5b}.
R^{5a} represents H;
R^{5b} represents H; or C₁₋₄ alkyl, saturated or unsaturated, branched or unbranched, unsubstituted; cyclohexyl, unsubstituted; or phenyl or benzyl, in each case unsubstituted or mono- or polysubstituted with one or more substituents each selected independently from the group consisting of F, Cl, Br, I, O—C₁₋₄ alkyl, CF₃, OCF₃ and C₁₋₄ alkyl; or
R^{5a} and R^{5b} together with the carbon atom connecting them form an unsubstituted C₃₋₁₀ cycloalkyl group, saturated or unsaturated;
B¹ represents C, CH, N, NR⁶, O or S;
B² represents C, CH, N, NR⁷, O or S; and
B³ represents C, CH, N, NR⁸, O or S;
wherein 1 or 2 of the variables B¹, B² and B³ represent one of the aforementioned heteroatoms or heteroatom groups;
D¹ represents N or CR⁹;
D² represents N or CR¹⁰;
D³ represents N or CR¹¹; and
D⁴ represents N or CR¹²,
wherein 0, 1 or 2 of the variables D¹, D², D³ and D⁴ represent N;
R⁶, R⁷ and R⁸ each independently represent H or C₁₋₄ alkyl, saturated, branched or unbranched, unsubstituted or mono- or polysubstituted; and
R⁹, R¹⁰, R¹¹ and R¹² are each selected independently from the group consisting of H; F; Cl; Br; I; CF₃; OCF₃; SCF₃; C₁₋₄ alkyl, O—C₁₋₄ alkyl and NH—S(═O)₂—C₁₋₄ alkyl, in which C₁₋₄ alkyl can be respectively saturated or unsaturated, branched or unbranched, and is unsubstituted.

13. A compound according to claim 1, selected from the group consisting of:
[1] 2-(1-methyl-1H-indol-3-yl)-N-((1-phenyl-3-(trifluoromethyl)-1H-pyrazol-5-yl)methyl)propanamide;
[2] N-((3-tert-butyl-1-(3-chlorophenyl)-1H-pyrazol-5-yl)methyl)-2-(1-methyl-1H-indol-3-yl)propanamide;
[3] N-((1-(3-methoxyphenyl)-3-(trifluoromethyl)-1H-pyrazol-5-yl)methyl)-2-(1-methyl-1H-indol-3-yl)propanamide;
[4] N-((1-cyclopentyl-3-(trifluoromethyl)-1H-pyrazol-5-yl)methyl)-2-(5-hydroxy-1-methyl-1H-indol-3-yl)propanamide;
[5] 2-(5-hydroxy-1-methyl-1H-indol-3-yl)-N-(1-m-tolyl-3-(trifluoromethyl)-1H-pyrazol-5-yl)methyl)propanamide;
[6] N-((3-tert-butyl-1-(pyridin-2-yl)-1H-pyrazol-5-yl)methyl)-2-(5-hydroxy-1-methyl-1H-indol-3-yl)propanamide;
[7] N-((1-(3-chlorophenyl)-4-methyl-3-(trifluoromethyl)-1H-pyrazol-5-yl)methyl)-2-(5-hydroxy-1-methyl-1H-indol-3-yl)propanamide;
[8] N-((1-(3-chlorophenyl)-3-(trifluoromethyl)-1H-pyrazol-5-yl)methyl)-2-(1-methyl-1H-indol-3-yl)propanamide;
[9] N-((1-(3-chlorophenyl)-3-(trifluoromethyl)-1H-pyrazol-5-yl)methyl)-2-(5-hydroxy-1-methyl-1H-indol-3-yl)acetamide;
[10] N-((1-(3-chlorophenyl)-3-(trifluoromethyl)-1H-pyrazol-5-yl)methyl)-2-(5-hydroxy-1-methyl-1H-indol-3-yl)propanamide;
[11] N-((3-tert-butyl-1-(3-chloro-4-fluorophenyl)-1H-pyrazol-5-yl)methyl)-2-(5-hydroxy-1-methyl-1H-indol-3-yl)propanamide;
[12] N-((1-(3-chlorophenyl)-3-cyclopropyl-1H-pyrazol-5-yl)methyl)-2-(5-hydroxy-1-methyl-1H-indol-3-yl)propanamide;
[13] N-((3-tert-butyl-1-(3-chlorophenyl)-1H-pyrazol-5-yl)methyl)-2-(5-methoxy-1-methyl-1H-indol-3-yl)acetamide;
[14] N-((3-tert-butyl-1-(3-chlorophenyl)-1H-pyrazol-5-yl)methyl)-2-(5-methoxy-1-methyl-1H-indol-3-yl)propanamide,
[15] N-((1-(cyclopropylmethyl)-3-(trifluoromethyl)-1H-pyrazol-5-yl)methyl)-2-(1-methyl-1H-indol-3-yl)propanamide;
[16] 1-((1-(3-chlorophenyl)-3-(trifluoromethyl)-1H-pyrazol-5-yl)methyl)-3-(1H-indazol-3-yl)urea;
[17] 1-((1-(3-chlorophenyl)-3-(trifluoromethyl)-1H-pyrazol-5-yl)methyl)-3-(1-methyl-1H-pyrazolo[3,4-b]pyridin-3-yl)urea;
[18] N-((3-tert-butyl-1-(3-chlorophenyl)-1H-1,2,4-triazol-5-yl)methyl)-2-(5-hydroxy-1-methyl-1H-indol-3-yl)acetamide;
[19] N-((3-tert-butyl-1-methyl-1H-pyrazol-5-yl)methyl)-2-(5-hydroxy-1-methyl-1H-indol-3-yl)propanamide;
[20] N-((3-tert-butyl-1-hexyl-1H-pyrazol-5-yl)methyl)-2-(5-hydroxy-1-methyl-1H-indol-3-yl)propanamide;
[21] N-((1-cyclohexyl-3-(trifluoromethyl)-1H-pyrazol-5-yl)methyl)-2-(5-hydroxy-1-methyl-1H-indol-3-yl)propanamide;
[22] 2-(5-hydroxy-1-methyl-1H-indol-3-yl)-N-((1-(tetrahydro-2H-pyran-4-yl)-3-(trifluoromethyl)-1H-pyrazol-5-yl)methyl)propanamide;
[23] 2-(5-hydroxy-1-methyl-1H-indol-3-yl)-N-((1-(oxetan-3-yl)-3-(trifluoromethyl)-1H-pyrazol-5-yl)methyl)propanamide;
[24] N-((3-tert-butyl-1-(3-fluorophenyl)-1H-pyrazol-5-yl)methyl)-2-(5-hydroxy-1-methyl-1H-indol-3-yl)propanamide;
[25] N-((3-tert-butyl-1-(4-methoxyphenyl)-1H-pyrazol-5-yl)methyl)-2-(5-hydroxy-1-methyl-1H-indol-3-yl)propanamide;
[26] N-((3-tert-butyl-1-(3-chloro-4-fluorophenyl)-1H-pyrazol-5-yl)methyl)-2-(5-hydroxy-1-methyl-1H-indol-3-yl)propanamide;
[27] 2-(5-hydroxy-1-methyl-1H-indol-3-yl)-N-((1-(4-methoxybenzyl)-3-(trifluoromethyl)-1H-pyrazol-5-yl)methyl)propanamide;
[28] 2-(5-hydroxy-1-methyl-1H-indol-3-yl)-N-((1-phenyl-3-(trifluoromethyl)-1H-pyrazol-5-yl)methyl)propanamide;
[29] N-((1-(4-fluorophenyl)-3-(trifluoromethyl)-1H-pyrazol-5-yl)methyl)-2-(5-hydroxy-1-methyl-1H-indol-3-yl)propanamide;
[30] 2-(5-hydroxy-1-methyl-1H-indol-3-yl)-N-((1-(3-hydroxyphenyl)-3-(trifluoromethyl)-1H-pyrazol-5-yl)methyl)propanamide;
[31] 2-(5-hydroxy-1-methyl-1H-indol-3-yl)-N-((1-(3-isopropylphenyl)-3-(trifluoromethyl)-1H-pyrazol-5-yl)methyl)propanamide;
[32] 2-(5-hydroxy-1-methyl-1H-indol-3-yl)-N-((1-(pyridin-3-yl)-3-(trifluoromethyl)-1H-pyrazol-5-yl)methyl)propanamide;
[33] 2-(5-hydroxy-1-methyl-1H-indol-3-yl)-N-((1-(pyrimidin-2-yl)-3-(trifluoromethyl)-1H-pyrazol-5-yl)methyl)propanamide;
[34] 1-((1-(3-chlorophenyl)-3-(trifluoromethyl)-1H-pyrazol-5-yl)methyl)-3-(1-methyl-1H-pyrrolo[2,3-c]pyridin-3-yl)urea;
[35] N-((1-(3-chlorophenyl)-3-(trifluoromethyl)-1H-pyrazol-5-yl)methyl)-2-(1,5-dimethyl-1H-indol-3-yl)propanamide;
[36] 1-((1-(3-chlorophenyl)-3-(trifluoromethyl)-1H-pyrazol-5-yl)methyl)-3-(5-fluoro-1-methyl-1H-indol-3-yl)urea;
[37] 1-((1-(3-chlorophenyl)-3-(trifluoromethyl)-1H-pyrazol-5-yl)methyl)-3-(7-methyl-7H-pyrrolo[2,3-d]pyrimidin-5-yl)urea;
[38] 1-((1-(3-chlorophenyl)-3-(trifluoromethyl)-1H-pyrazol-5-yl)methyl)-3-(5-(dimethylamino)-1-methyl-1H-indol-3-yl)urea;
[40] 1-((1-(3-chlorophenyl)-3-(trifluoromethyl)-1H-pyrazol-5-yl)methyl)-3-(5-hydroxybenzo[d]oxazol-2-yl)urea;
[41] 1-((1-(3-chlorophenyl)-3-(trifluoromethyl)-1H-pyrazol-5-yl)methyl)-3-(6-hydroxybenzo[d]oxazol-2-yl)urea;
[42] 1-((1-(3-chlorophenyl)-3-(trifluoromethyl)-1H-pyrazol-5-yl)methyl)-3-(4-hydroxybenzo[d]oxazol-2-yl)urea;
[43] 1-(1H-benzo[d]imidazol-2-yl)-3-((1-(3-chlorophenyl)-3-(trifluoromethyl)-1H-pyrazol-5-yl)methyl)urea;
[44] 1-(1H-benzo[d]imidazol-2-yl)-3-((1-(3-chlorophenyl)-3-(trifluoromethyl)-1H-pyrazol-5-yl)methyl)urea;
[45] 1-((1-(3-chlorophenyl)-3-(trifluoromethyl)-1H-pyrazol-5-yl)methyl)-3-(1-methyl-1H-benzo[d]imidazol-2-yl)urea;
[46] 1-(6-chloro-1H-benzo[d]imidazol-2-yl)-3-((1-(3-chlorophenyl)-3-(trifluoromethyl)-1H-pyrazol-5-yl)methyl)urea;
[47] 1-(5-chlorobenzo[d]oxazol-2-yl)-3-((1-(3-chlorophenyl)-3-(trifluoromethyl)-1H-pyrazol-5-yl)methyl)urea;
[48] 1-((1-(3-chlorophenyl)-3-(trifluoromethyl)-1H-pyrazol-5-yl)methyl)-3-(6-methoxybenzo[d]thiazol-2-yl)urea, and
[49] 1-((1-(3-chlorophenyl)-3-(trifluoromethyl)-1H-pyrazol-5-yl)methyl)-3-(6-(methylsulfonyl)benzo[d]thiazol-2-yl)urea;
or a salt thereof with a physiologically compatible acid or base.

14. A pharmaceutical composition comprising a compound according to claim 1 and at least one pharmaceutically acceptable carrier or auxiliary substance.

15. A method of treating or inhibiting a disorder selected from the group consisting of pain; hyperalgesia; allodynia; causalgia; migraine; depression; nervous affection; axonal injuries; neurodegenerative diseases; cognitive dysfunctions; epilepsy; respiratory diseases; coughs; urinary incontinence; overactive bladder; disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations; diarrhea; pruritus; osteoporosis; arthritis; osteoarthritis; rheumatic diseases; eating disorders; medication dependency; misuse of medication; withdrawal symptoms in medication dependency; development of tolerance to medication; drug dependency; misuse of drugs; withdrawal symptoms in drug dependency; alcohol dependency; misuse of alcohol and withdrawal symptoms in alcohol dependency; or for effecting diuresis; antinatriuresis; influencing the cardiovascular system; increasing vigilance; treating wounds and/or burns; treating severed nerves; increasing libido; modulating movement activity; effecting anxiolysis; local anaesthesia or inhibiting undesired side effects triggered by the administration of a vanilloid receptor 1 agonist, in a subject in need thereof, said method comprising administering to said subject a pharmaceutically effective amount of a compound according to claim 1.

16. A method according to claim 15, wherein said disorder is selected from the group consisting of pain selected from the group consisting of acute pain, chronic pain, neuropathic pain, visceral pain and joint pain; a neurodegenerative disease selected from the group consisting of multiple sclerosis, Alzheimer's disease, Parkinson's disease and Huntington's disease; a memory disorder; a respiratory disease selected from the group consisting of asthma, bronchitis and pulmonary inflammation; an inflammation of the intestine, the eyes, the bladder, the skin or the nasal mucous membrane; an eating disorder selected from the group consisting of bulimia, cachexia, anorexia and obesity; development of tolerance to natural or synthetic opioids; or for inhibiting an undesirable side effect selected from the group consisting of hyperthermia, hypertension and bronchoconstriction, triggered by the administration of a vanilloid receptor 1 agonist selected from the group consisting of capsaicin, resiniferatoxin, olvanil, arvanil, SDZ-249665, SDZ-249482, nuvanil and capsavanil.
